(19) United States
(12) Patent Application Publication
(10) Pub. No.: US 2026/0109713 A1
(43) Pub. Date: Apr. 23, 2026

(54) SIRTUIN MODULATING COMPOUNDS, INCLUDING SIRTUIN ACTIVATING COMPOUNDS, AND APPLICATIONS THEREOF

(71) Applicant: CODAGEN BIOSCIENCES, INC., Mt. Laurel, NJ (US)

(72) Inventors: Célina Reverdy, Chilly-Mazarin (FR); Xiangying Guan, Mt. Laurel, NJ (US); Samir Roy, Mt. Laurel, NJ (US); Santu Chall, Mt. Laurel, NJ (US); Gauthier Errasti, Orsay (FR); Thomas Delacroix, Mennecy (FR); Raj Chakrabarti, Moorestown, NJ (US)

(21) Appl. No.: 18/876,032

(22) PCT Filed: Jun. 19, 2023
(86) PCT No.: PCT/US2023/025673

§ 371 Date: Dec. 17, 2024

Related U.S. Application Data
(60) Provisional application No. 63/353,248, filed on Jun. 17, 2022.

(51) Int. Cl. C07D 513/04; C07C 259/18; C07D 239/69; C07D 413/12; C07D 417/12; C07D 487/04

(52) U.S. Cl. CPC C07D 513/04; C07C 259/18; C07D 239/69; C07D 413/12; C07D 417/12; C07D 487/04

(57) ABSTRACT

In one aspect, aryl carboximidamide compounds, comprising a carboximidamide molecular substructure, and modulating mammalian sirtuin enzymes (SIRT1-7) are described herein, and, in particular, to compounds for the modulation of SIRT3. Novel small molecules that modulate sirtuin activities were developed employing DNA encoded Library technology (ELT). Modulation of sirtuin activity includes sirtuin activation and/or sirtuin inhibition.

### RELATED APPLICATION DATA

This application is a U.S. National Phase of PCT/US23/25673 filed Jun. 19, 2023, which claims priority pursuant to 35 U.S.C. § 119(e) to U.S. Provisional Patent Application Ser. No. 63/353,248 filed Jun. 17, 2022 which is incorporated herein by reference in its entirety.

### FIELD

The present application addresses compounds exhibiting sirtuin modulating functionality and, in particular, to compounds for the modulation of SIRT3.

### BACKGROUND

Sirtuin (silent information regulator) enzymes, which catalyze NAD⁺-dependent protein post-translational modifications, have emerged as critical regulators of many cellular pathways. In particular, these enzymes protect against age-related diseases and serve as key mediators of longevity in evolutionarily distant organismic models. Sirtuins are NAD⁺-dependent lysine deacylases, requiring the cofactor NAD⁺ to cleave acyl groups from lysine side chains of their substrate proteins.

A thorough understanding of sirtuin chemistry is not only of fundamental importance, but also of considerable medicinal importance, since there is enormous current interest to develop new mechanism-based sirtuin modulators. Its overall catalytic process has been suggested to proceed in two consecutive stages. The initial stage involves the cleavage of the nicotinamide moiety of NAD⁺ and the nucleophilic attack of the acetyl-Lys side chain of the protein substrate to form a positively charged O-alkylimidate intermediate. Nicotinamide-induced reversal of the intermediate (the so-called base exchange reaction) causes reformation of NAD⁺ and acetyl-Lys protein. The energetics of this reversible reaction affects both the potency of nicotinamide (NAM) inhibition of sirtuins and the Michaelis constant for NAD⁺ (K_{m,NAD+}). The second stage of sirtuin catalysis, which includes the rate determining step, involves four successive steps that culminate in deacetylation of the Lys side chain of the protein substrate and the formation of O-acetyl ADP ribose coproduct.

Recently, in order to combat old age, intense interest has developed in the activation of the seven mammalian sirtuin enzymes (SIRT1-7). Compared to enzyme inhibitors, which constitute the vast majority of today's drugs, enzyme activators have considerable advantages. However, they are much more difficult to design, because enzymatic catalysis has been optimized over billions of years of evolution. Prior work on sirtuin activation has focused exclusively on experimental screening, with an emphasis on allosteric activation of the SIRT1 enzyme. Indeed, small molecule allosteric activators of SIRT1 have been demonstrated to induce lifespan extension in model organisms such as mice. Allosteric activation is one of four known modes by which small molecules can activate enzymes. They function by decreasing the dissociation constant for the substrate (the acetylated protein dissociation constant K_{d,Ac—Pr} for sirtuins).

Almost all known sirtuin activators allosterically target SIRT1 and do not bind in the active site. However, allosteric activators only work with certain substrates of SIRT1. It is now known that other sirtuins, including SIRT2, SIRT3 and SIRT6, play significant roles in regulating mammalian longevity. General strategies for the activation of any mammalian sirtuin (including activation of SIRT1 for other substrates) are hence of central importance, but not understood. In general, allosteric activation to decrease substrate K_{d} will not be an option for enzyme activation, rending mechanism-based activation important.

Foundations for the rational design of mechanism-based activators have been lacking. Several types of mechanism-based sirtuin inhibitors have been reported recently in the literature, including Ex-527. However, mechanism-based activation has proven far more elusive, due to the difficulty in screening for the balance of properties needed for a modulator to bind the active site and accelerate catalysis. While there are many ways to inhibit an enzyme's mechanism, there are far fewer ways to activate it. Only a dozen or so distinct classes of small molecule enzyme activators are currently known, with only four known modes of activation across all families of enzymes. None of those modes of activation exploit the unique catalytic reaction mechanisms of the target enzymes.

### SUMMARY

In one aspect, compounds modulating sirtuin activity are described herein. Modulation of sirtuin activity includes sirtuin activation and/or sirtuin inhibition. In some embodiments, a sirtuin modulating compound and/or salt thereof is of Formula I:

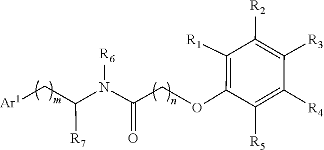

wherein Ar¹ is aryl or heteroaryl, R₁-R₇ are independently selected from the group consisting of hydrogen, alkyl, alkenyl, heteroalkyl, cycloalkyl, heterocycloalkyl, —C(O)R₈, alkoxy, halo, nitryl (−NO₂), and hydroxy, wherein the Ar¹, alkyl, alkenyl, heteroalkyl, cycloalkyl, heterocycloalkyl, aryl, heteroaryl are optionally substituted with one or more substituents selected from the group consisting of (C₁-C₁₀)-alkyl, (C₁-C₁₀)-alkenyl, alkoxy, halo, amine, and hydroxy; and wherein R₈ is selected from the group consisting of alkyl, alkenyl, and NR₉R₁₀, wherein R₉ and R₁₀ are independently selected from the group consisting of hydrogen, alkyl, and cycloalkyl; and wherein m and n are integers each having a value independently selected from 0 to 10. In some embodiments, a compound of Formula I and/or salt thereof is:

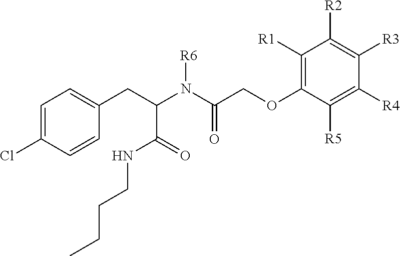

In another aspect, a sirtuin modulating compound and/or salt thereof is of Formula II:

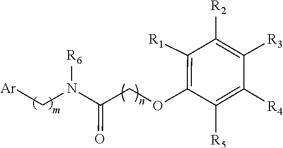

wherein Ar is aryl or heteroaryl, R₁-R₆ are independently selected from the group consisting of hydrogen, alkyl, alkenyl, heteroalkyl, cycloalkyl, heterocycloalkyl, —C(O)R₇, alkoxy, halo, nitryl (—NO₂), and hydroxy, wherein the Ar¹, alkyl, alkenyl, heteroalkyl, cycloalkyl, heterocycloalkyl, aryl, heteroaryl are optionally substituted with one or more substituents selected from the group consisting of (C₁-C₁₀)-alkyl, (C₁-C₁₀)-alkenyl, alkoxy, halo, amine, -alkoxy-amide, and hydroxy; and wherein R₇ is selected from the group consisting of alkyl, alkenyl, and NR₈R₉, wherein R₈ and R₉ are independently selected from the group consisting of hydrogen, alkyl, and cycloalkyl; and wherein m and n are integers each having a value independently selected from 0 to 10. In some embodiments, for example, a compound of Formula II and/or salt thereof is:

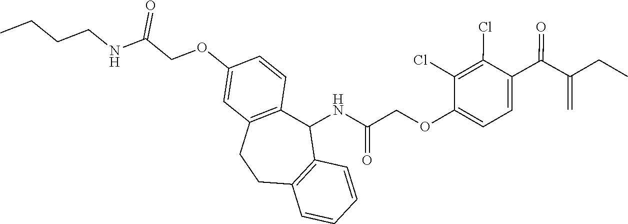 [SMILES: C=C(CC)C(=O)c1ccc(OCC(=O)NC2c3ccccc3CCc3cc(OCC(=O)NCCCC)ccc32)c(Cl)c1Cl]

In another embodiment, a compound of Formula II and/or salt thereof is:

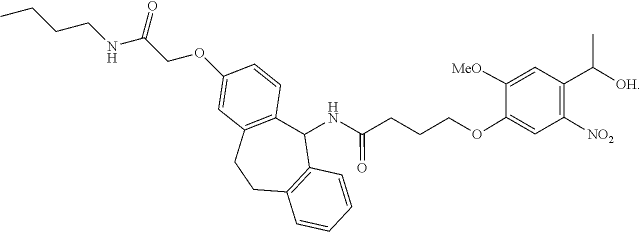 [SMILES: CCCCNC(=O)COc1ccc2c(c1)CCc1ccccc1C2NC(=O)CCCOc1cc([N+](=O)[O-])c(C(C)O)cc1OC]

In another aspect, a sirtuin modulating compound and/or salt thereof is of Formula III:

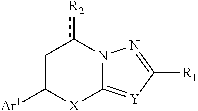

wherein Ar¹ is aryl or heteroaryl, R¹ is selected from the group consisting of hydrogen, alkyl, alkenyl, cycloalkyl, heterocycloalkyl, aryl, heteroaryl, amine, and —C(O)NR₃R₄, wherein the Ar¹ alkyl, alkenyl, alkynyl, acyl, alkoxy, alkenyloxy, cycloalkyloxy, cycloalkenyl, alkyl amine, acetamide, acetylamine, cycloalkyl, heterocycloalkyl, aryl, heteroaryl, and amine are optionally substituted with one or more substituents selected from the group consisting of (C₁-C₁₀)-alkyl, (C₁-C₁₀)-alkenyl, alkoxy, halo, amine, and hydroxy; and wherein R₃ and R₄ are independently selected from the group consisting of hydrogen, alkyl, alkenyl, aryl, and heteroaryl, and wherein R² is selected from the group consisting of hydrogen, alkyl, fluoroalkyl, alkenyl, aryl, heteroaryl, alkynyl, acyl, alkoxy, alkenyloxy, cycloalkyloxy, cycloalkenyl, amine, alkyl amine, and O, and wherein Y is selected from the group consisting of N and CH, and wherein X is selected from the group consisting of NH, S and O. In some embodiments, a compound of Formula III and/or salt thereof includes:

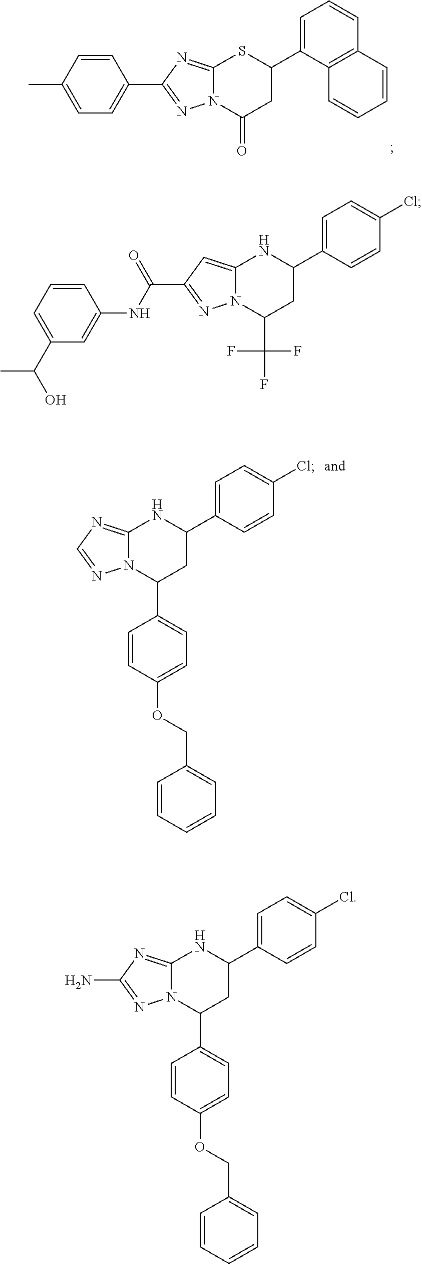 [SMILES: Cc1ccc(-c2nc3n(n2)C(=O)CC(c2cccc4ccccc24)S3)cc1 ; CC(O)c1cccc(NC(=O)c2cc3n(n2)C(C(F)(F)F)CC(c2ccc(Cl)cc2)N3)c1 ; Clc1ccc(C2CC(c3ccc(OCc4ccccc4)cc3)n3ncnc3N2)cc1 ; "and" ; Nc1nc2n(n1)C(c1ccc(OCc3ccccc3)cc1)CC(c1ccc(Cl)cc1)N2]

In another aspect, a sirtuin modulating compound and/or salt thereof is of Formula IV:

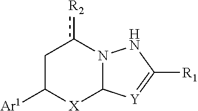

wherein Ar¹ is aryl or heteroaryl, R¹ is selected from the group consisting of hydrogen, alkyl, alkenyl, cycloalkyl, heterocycloalkyl, aryl, heteroaryl, amine, and —C(O)NR₃R₄, wherein the Ar¹ alkyl, alkenyl, alkynyl, acyl, alkoxy, alkenyloxy, cycloalkyloxy, cycloalkenyl, alkyl amine, acetamide, acetylamine, cycloalkyl, heterocycloalkyl, aryl, heteroaryl, and amine are optionally substituted with one or more substituents selected from the group consisting of (C₁-C₁₀)-alkyl, (C₁-C₁₀)-alkenyl, alkoxy, halo, amine, and hydroxy; and wherein R₃ and R₄ are independently selected from the group consisting of hydrogen, alkyl, alkenyl, aryl, and heteroaryl, and wherein R² is selected from the group consisting of hydrogen, alkyl, fluoroalkyl, alkenyl, aryl, heteroaryl, alkynyl, acyl, alkoxy, alkenyloxy, cycloalkyloxy, cycloalkenyl, amine, alkyl amine, and O, and wherein Y is selected from the group consisting of N and CH, and wherein X is selected from the group consisting of NH, S and O. In some embodiments, a compound of Formula III and/or salt thereof includes:

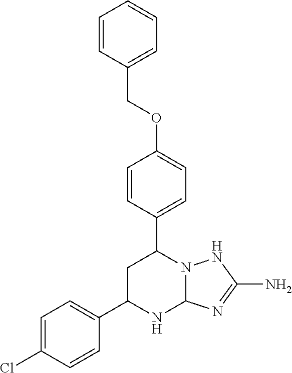 [SMILES: NC1=NC2NC(c3ccc(Cl)cc3)CC(c3ccc(OCc4ccccc4)cc3)N2N1]

In a further aspect, a sirtuin modulating compound and/or salt thereof is of Formula V:

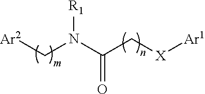

wherein R₁ is selected from the group consisting of hydrogen, alkyl, heteroalkyl, cycloalkyl, alkenyl, alkynyl, acyl, alkoxy, alkenyloxy, cycloalkyloxy, cycloalkenyl, and heterocycloalkyl; and wherein Ar¹ and Ar² are independently selected from aryl and heteroaryl, wherein the aryl and heteroaryl are optionally substituted with one or more substituents selected from the group consisting of (C₁-C₁₀)-alkyl, (C₁-C₁₀)-alkenyl, alkoxy, halo, amine, sulfonyl-alkyl, and hydroxyl; and wherein X is selected from the group consisting of O, S and N; and wherein m and n are integers each having a value independently selected from 0 to 10.

In some embodiments, a compound of Formula V and/or salt thereof is selected from one of the following:

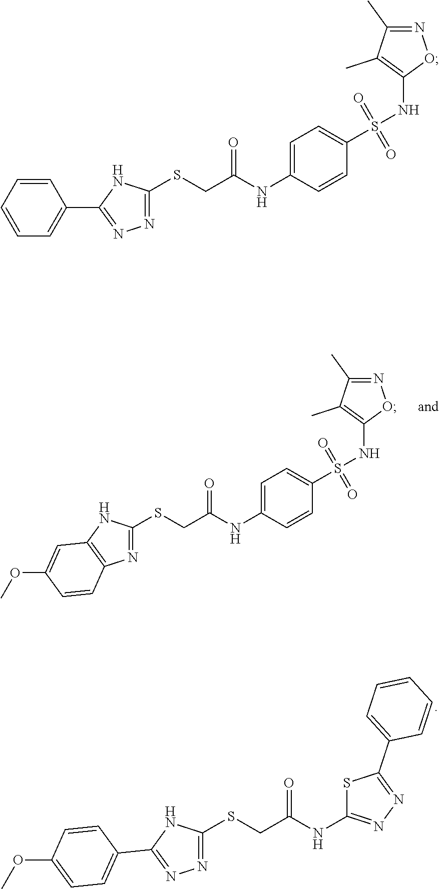 [SMILES: Cc1noc(NS(=O)(=O)c2ccc(NC(=O)CSc3nnc(-c4ccccc4)[nH]3)cc2)c1C ; COc1ccc2nc(SCC(=O)Nc3ccc(S(=O)(=O)Nc4onc(C)c4C)cc3)[nH]c2c1 ; COc1ccc(-c2nnc(SCC(=O)Nc3nnc(-c4ccccc4)s3)[nH]2)cc1]

In another aspect, a sirtuin modulating compound and/or salt thereof is of Formula VI:

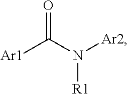

wherein R₁ is selected from the group consisting of hydrogen, alkyl, heteroalkyl, cycloalkyl, alkenyl, alkynyl, acyl, alkoxy, alkenyloxy, cycloalkyloxy, cycloalkenyl, and heterocycloalkyl; and wherein Ar¹ and Ar² are independently selected from aryl and heteroaryl, wherein the aryl and heteroaryl are optionally substituted with one or more substituents selected from the group consisting of (C₁-C₁₀)-alkyl, (C₁-C₁₀)-alkenyl, alkoxy, halo, amine, sulfonyl-alkyl, acyl, amide, acetylamine, alkylamine, and hydroxyl. In some embodiments, a compound of Formula VI and/or salt thereof is selected from one of the following:

In some embodiments, a compound of Formula VI and/or salt thereof is selected from one of the following:

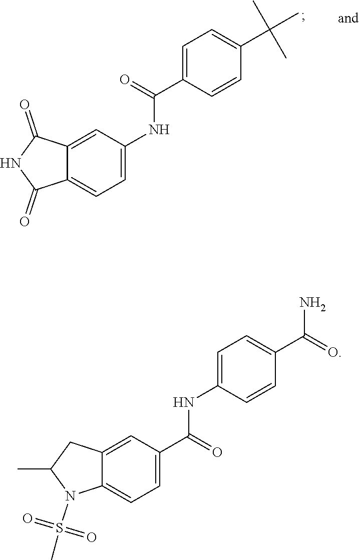

In another aspect, a sirtuin modulating compound and/or salt thereof is of Formula VII:

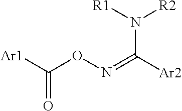

wherein R₁ and R₂ are independently selected from the group consisting of hydrogen, alkyl, heteroalkyl, cycloalkyl, and heterocycloalkyl; and wherein Ar1 and Ar2 are independently selected from aryl and heteroaryl, wherein the aryl and heteroaryl are optionally substituted with one or more substituents selected from the group consisting of (C₁-C₁₀)-alkyl, (C₁-C₁₀)-alkenyl, alkoxy, halo, amine, nitro, sulfonyl-alkyl, acyl, amide, acetylamine, alkyl amine, alkynyl, and hydroxyl. In some embodiments, a compound of Formula VII and/or salt thereof is:

In some embodiments, a compound of Formula VII and/or salt thereof is:

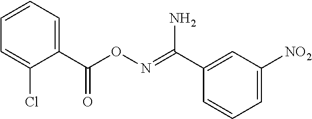 [SMILES: N/C(=N\OC(=O)c1ccccc1Cl)c1cccc([N+](=O)[O-])c1]

In another aspect, a sirtuin modulating compound and/or salt thereof is of Formula VIII:

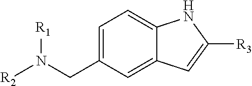

wherein R₁-R₃ are independently selected from the group consisting of hydrogen, alkyl, heteroalkyl, cycloalkyl, alkenyl, alkynyl, acyl, and heterocycloalkyl, wherein the alkyl, heteroalkyl, cycloalkyl, and heterocycloalkyl are optionally substituted with one or more substituents selected from the group consisting of alkoxy, halo, amine, nitro, and hydroxyl. In some embodiments, a compound of Formula VIII and/or salt thereof is.

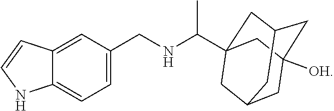 [SMILES: CC(NCc1ccc2[nH]ccc2c1)C12CC3CC(CC(O)(C3)C1)C2]

In another aspect, a sirtuin modulating compound and/or salt thereof is of Formula IX:

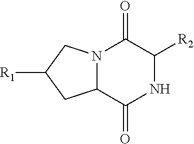

wherein R₁ and R₂ are independently selected from the group consisting of hydrogen, alkyl, alkenyl, alkynyl, acyl, heteroalkyl, cycloalkyl, heterocycloalkyl, amide, and keto, wherein the alkyl, heteroalkyl, cycloalkyl, heterocycloalkyl, amide, and keto are optionally substituted with one or more substituents selected from the group consisting of aryl, heteroaryl, alkoxy, halo, amine, nitro, and hydroxyl. In some embodiments, a compound of Formula IX and/or salt thereof is:

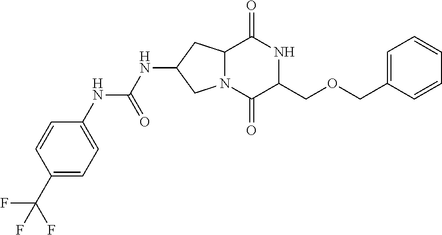 [SMILES: O=C(Nc1ccc(C(F)(F)F)cc1)NC1CC2C(=O)NC(COCc3ccccc3)C(=O)N2C1]

In another aspect, a sirtuin modulating compound and/or salt thereof is of Formula X:

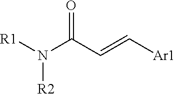

wherein R₁ and R₂ are independently selected from the group consisting of hydrogen, alkyl, heteroalkyl, cycloalkyl, alkenyl, alkynyl, acyl, and heterocycloalkyl; and wherein Ar¹ is selected from aryl and heteroaryl, wherein the cycloalkyl, heterocycloalkyl, aryl and heteroaryl are optionally substituted with one or more substituents selected from the group consisting of (C₁-C₁₀)-alkyl, (C₁-C₁₀)-alkenyl, aryl, alkoxy, halo, amine, nitro, sulfonyl-alkyl, acyl, amide, acetylamine, alkyl amine, alkynyl, and hydroxyl. In some embodiments, a compound of Formula X and/or salt thereof is:

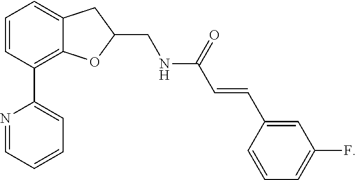 [SMILES: O=C(/C=C/c1cccc(F)c1)NCC1Cc2cccc(-c3ccccn3)c2O1]

In another aspect, a sirtuin modulating compound and/or salt thereof is of Formula XI:

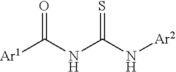

wherein Ar¹ and Ar² are independently selected from aryl and heteroaryl, wherein the aryl and heteroaryl are optionally substituted with one or more substituents selected from the group consisting of (C₁-C₁₀)-alkyl, (C₁-C₁₀)-alkenyl, alkoxy, halo, amine, sulfonyl-alkyl, acyl, amide, acetylamine, alkyl amine, alkynyl, and hydroxyl. In some embodiments, a compound of Formula XI and/or salt thereof is:

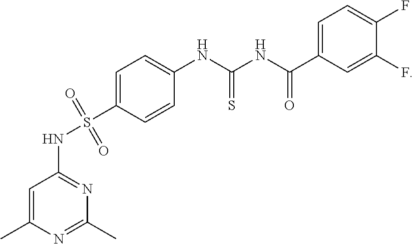 [SMILES: Cc1cc(NS(=O)(=O)c2ccc(NC(=S)NC(=O)c3ccc(F)c(F)c3)cc2)nc(C)n1]

In another aspect, a sirtuin modulating compound and/or salt thereof is of Formula XII:

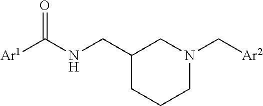

wherein Ar¹ and Ar² are independently selected from aryl and heteroaryl, wherein the aryl and heteroaryl are optionally substituted with one or more substituents selected from the group consisting of (C₁-C₁₀)-alkyl, (C₁-C₁₀)-alkenyl, alkoxy, halo, amine, sulfonyl-alkyl, acyl, amide, acetylamine, alkyl amine, alkynyl, and hydroxyl. In some embodiments, a compound of Formula XII and/or salt thereof is:

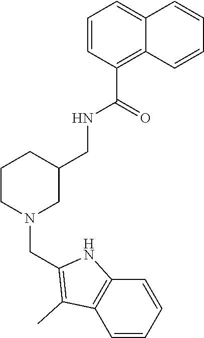 [SMILES: Cc1c(CN2CCCC(CNC(=O)c3cccc4ccccc34)C2)[nH]c2ccccc12]

In another aspect, a sirtuin modulating compound and/or salt thereof is of Formula XIII:

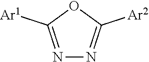

wherein Ar¹ and Ar² are independently selected from aryl and heteroaryl, wherein the aryl and heteroaryl are optionally substituted with one or more substituents selected from the group consisting of (C₁-C₁₀)-alkyl, (C₁-C₁₀)-alkenyl, alkoxy, halo, amine, sulfonyl-alkyl, acyl, amide, acetylamine, alkyl amine, alkynyl, and hydroxyl. In some embodiments, a compound of Formula XIII and/or salt thereof is:

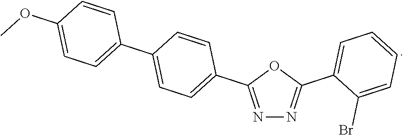 [SMILES: COc1ccc(-c2ccc(-c3nnc(-c4ccccc4Br)o3)cc2)cc1]

In another aspect, a sirtuin modulating compound and/or salt thereof is of Formula XIV:

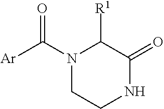

wherein R¹ is selected from the group consisting of hydrogen, alkyl, heteroalkyl, cycloalkyl, alkenyl, alkynyl, acyl, heteroalkenyl, heteroalkynyl, and heterocycloalkyl; and wherein Ar is selected from aryl and heteroaryl, wherein the aryl and heteroaryl are optionally substituted with one or more substituents selected from the group consisting of (C₁-C₁₀)-alkyl, (C₁-C₁₀)-alkenyl, alkoxy, halo, amine, sulfonyl-alkyl, acyl, amide, acetylamine, alkyl amine, alkynyl, and hydroxyl. In some embodiments, a compound of Formula XIV is:

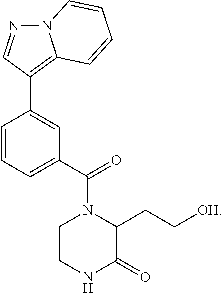 [SMILES: O=C1NCCN(C(=O)c2cccc(-c3cnn4ccccc34)c2)C1CCO]

As described further herein, compounds of Formulas I-XIV and/or salts thereof can modulate SIRT3, in some embodiments. Moreover, methods of treating patients are also described herein. In some embodiments, a method comprises administering a therapeutically effective amount of a compound selected from Formulas III-XIV herein to a patient, wherein the compound raises NAD⁺ in the patient. In some embodiments, administration of a compound described herein enhances cellular metabolism and/or energy production in the patient. Compounds of Formulas III-XIV, in some embodiments, can rejuvenate the activity of the major mitochondrial sirtuin levels observed in youth by increasing sirtuin sensitivity to NAD⁺ which provides effective treatment to age-related diseases, including mitochondrial diseases.

These and other embodiments are further described in the following detailed description.

### BRIEF DESCRIPTION OF THE DRAWINGS

FIG. 1. Frequency of all the compounds in the native library. All the derivatives of the 20 amino acids show a similar distribution over its 194 carboxylic acid counterparts in the library.

FIG. 2. SIRT3 selection in the presence of Carba-NAD and acetylated peptide. The derivatives of AC71 and AC92 show the highest enrichment. Among the other, few derivatives of AC40 and AC56 also show significant enrichment. The Y-axis represents the enrichment calculated for each compound as the ratio of their frequencies in the target selection to their frequencies in the no target selection.

FIG. 3. SIRT3 selection in the presence of OAADPr and deacetylated peptide. The derivatives of AC71 and AC92 show the highest enrichment. Among the other, few derivatives of AC40 and AC56 also show significant enrichment. The Y-axis represents the enrichment calculated for each compound as the ratio of their frequencies in the target selection to their frequencies in the no target selection.

FIG. 4. Effect of top ranked hit compounds on Sirt3 deacetylation activity. Bar diagram of % control by different compounds ([NAD⁺]=1 mM, [FdL2 peptide]=50 μM, [cpd]=1, 10, 50 μM or maxi, [E]₀/[NAD]₀=0.000245, Time point=30 min, n=2).

FIG. 5. Sirt3 activation by top ranked hit compounds. Bar diagram of % control (HKL) by different compounds ([NAD⁺]=50 μM, [MnSOD K122]=600 μM, [cpd]=10 μM or maxi, [E]₀/[NAD]₀=0.1223, Time point=2 min, n=3).

FIG. 6. DNA-tagged and C-terminal amidated forms for selected active and inactive ligands identified by affinity selection assay. (A) Structure of DNA-tagged version of AA12-AC71, an active ligand; (B) Structure of C-terminal amidated version of AA12-AC71; (C) Structure of DNA-tagged version of AA20-AC92, another active ligand; (D) Structure of C-terminal amidated version of AA20-AC92; (E) Structure of DNA-tagged version of AA5-AC117, an inactive ligand; (F) Structure of C-terminal amidated version of AA5-CA117. Compounds depicted in B, D and F were used for all ligand-docking studies, instead of those in A, C and E.

FIG. 7. Location of Site 1 in 4BVG and top poses of docked hits. (A) Location of first putative atypical site, defined by pseudo-atoms, in 4BVG is almost antipodal to well-known internal site. Lining residues are highlighted using cyan. (B) Close-up of top docked poses of AA5/7/11/12-AC92 at putative atypical site. (C) Overlay for top docked poses of AA5/7/11/12-AC92 shows the high degree of overlap for pharmacophores. (D) Overlay for top docked poses of AA5/7/11/12-AC71 also show a high degree of overlap for pharmacophores at atypical Site 1 in 4BVG.

FIG. 8. Docking pose of hit compound 6875218 (FIG. 13) in SIRT3 open (4FVT) and close (4BVG) loop conformations. The figure shows the ternary complex of SIRT3 bound to Ac-ACS peptide and Carba-NAD. The protein is depicted in cyan, peptide in green, NAD⁺ in blue, and the compound are in pink. The protein residues that form interactions are shown in white sticks. The inter-molecular hydrogen bonds are shown in yellow dashes with corresponding distance labels.

FIG. 9. The modulation effect of the top 70 selected compounds under steady state conditions (N=3).

FIG. 10. The modulation effect of the top 20 selected compounds under non-steady state conditions (N=3).

FIG. 11. Common structural patterns in Sirt3 activator hit compounds. ChemBridge ID (A) 6068318 (FIG. 12), (B) 5749820, (C) 5761865, (D) 7965907 (FIG. 31). All the compounds presented here appear to share certain common structural features specifically a scaffold made up of a large bicyclic aromatic ring link to a six-member aromatic by an amide group.

FIGS. 12-295. illustrate various compounds of Formulas 3, 5-11, and 14 described herein.

FIG. 296. is a schematic illustrated the compound screening process employed in some embodiments of the present application.

FIGS. 297(A)-(D). Effect of VS hit compounds on SIRT3 deacetylation activity under steady state conditions: dose-response curves and time series of enzyme activation. (A) Dose-response curves for 3 VS hit compounds under [E]₀/[NAD⁺]₀=0.00157, in the presence of 1 mM NAD⁺ and 50 μM MnSOD peptide, t=30 min, N=3. (B) Bar diagram of % control by 2 VS hit compounds (20 μM 5329973 or 1 μM 5689785) on SIRT3 deacetylation activity under steady state conditions where [NAD⁺]=100 μM, [MnSOD K122]=600 μM, [E]₀/[NAD⁺]₀=0.00157, t=10 min, N=2. (* P<0.001). Plots of product formation vs. time in the presence and absence of (C) 20 μM 5329973 for [NAD⁺]=100 and 500 μM, [MnSOD K122]=600 μM, [E]₀/[NAD⁺]₀=0.00157, (N=2); (D) 1 μM 5689785 for [NAD⁺]=100 and 500 μM, [MnSOD K122]=600 μM, [E]₀/[NAD⁺]₀=0.00157, (N=2).

FIGS. 298(A)-(E). Binding affinity measurements for complexes in the sirtuin reaction mechanism. (A, C, E) Pathways in the sirtuin reaction network; E, enzyme; Ac—Pr, acetylated peptide substrate; NAD, nicotinamide adenine dinucleotide; A, modulator (HKL or hit compound 5329973); NAM, nicotinamide adenine mononucleotide. (B) Carba-NAD binding in the ternary complex: effect of mechanism-based modulator HKL. Binding of carba NAD (c-NAD) to Sirt3. Ac-MnSOD complex, in presence and absence of 6.25 μM HKL measured using MST. (D) HKL binding: effect of NAM. Binding of HKL to the Sirt3.Ac-MnSOD complex, in presence and absence of NAM, measured using MST. (F) Hit compound 5329973 binding to the apo enzyme Sirt3.

FIGS. 299(A)-(D). Steady state kinetic characterization of hSIRT3¹⁰²⁻³⁹⁹ deacetylation of MnSOD substrate by non-steady state activator HKL and steady state activators. Double reciprocal plots for deacetylation initial rate measurements in the presence and absence of HKL at (A) [NAM]=0 μM. (B) [NAM]=5 mM. Double reciprocal plots for SIRT3 deacetylation initial rate measurements in the presence and absence of (C) 20 μM 5329973 and (D) 1 μM 5699785. The initial rate was measured under [MnSOD K122]=600 μM with different [NAD⁺] concentrations at series of time points (N=2).

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Definitions

The term “alkyl” as used herein, alone or in combination, refers to a straight or branched saturated hydrocarbon group optionally substituted with one or more substituents. For example, an alkyl can be C₁-C₃₀ or C₁-C₁₈.

The term “alkenyl” as used herein, alone or in combination, refers to a straight or branched chain hydrocarbon group having at least one carbon-carbon double bond and optionally substituted with one or more substituents

The term “aryl” as used herein, alone or in combination, refers to an aromatic monocyclic or multicyclic ring system optionally substituted with one or more ring substituents. The term “aryl” includes fused ring systems and non-fused ring systems, where non-fused ring systems include alkylene and sulfonamide linking moieties between the rings.

The term “heteroaryl” as used herein, alone or in combination, refers to an aromatic monocyclic or multicyclic ring system in which one or more of the ring atoms is an element other than carbon, such as nitrogen, oxygen and/or sulfur. The term “heteroaryl” includes fused ring systems and non-fused ring systems, where non-fused ring systems include alkylene and sulfonamide linking moieties between the rings. For multicyclic ring systems, there is no requirement that each ring be aromatic.

The term “cycloalkyl” as used herein, alone or in combination, refers to a non-aromatic, mono- or multicyclic ring system optionally substituted with one or more ring substituents.

The term “heterocycloalkyl” as used herein, alone or in combination, refers to a non-aromatic, mono- or multicyclic ring system in which one or more of the atoms in the ring system is an element other than carbon, such as nitrogen, oxygen or sulfur, alone or in combination, and wherein the ring system is optionally substituted with one or more ring substituents.

The term “heteroalkyl” as used herein, alone or in combination, refers to an alkyl moiety as defined above, having one or more carbon atoms in the chain, for example one, two or three carbon atoms, replaced with one or more heteroatoms, which may be the same or different, where the point of attachment to the remainder of the molecule is through a carbon atom of the heteroalkyl radical.

The term “alkoxy” as used herein, alone or in combination, refers to the moiety RO—, where R is alkyl or alkenyl defined above.

The term “halo” as used herein, alone or in combination, refers to elements of Group VIIA of the Periodic Table (halogens). Depending on chemical environment, halo can be in a neutral or anionic state.

### I. DNA Encoded Library Selection (Formulas I and II)

In the effort to develop novel small molecules that modulate sirtuin activities, DNA encoded Library technology (ELT) was employed. Such a technique provided a robust hit identification approach that used large collections of diverse DNA-encoded small molecule libraries which are screened for their affinity against a desired protein target. The technology provided an efficient method to screen a broad chemical space of structures. It is also an attractive strategy as it requires negligible amounts of target protein to do the selection experiments and it identifies ligands regardless of their functional activity.

A 3880-member DNA encoded library based on the design defined by Mannocci et al. and consisting of the coupling of 20 amino acids with 194 carboxylic acids was produced and screened. The affinity mediated selection against SIRT3 was performed both under the presence of Carba-NAD (a stable NAD⁺ analog) and OAADPR to identify compounds that differentially bind to SIRT3 under such conditions. The Carba-NAD or OAADPr along with the acetylated or deacetylated peptide substrate is necessary to exhibit a functional SIRT3 conformation.

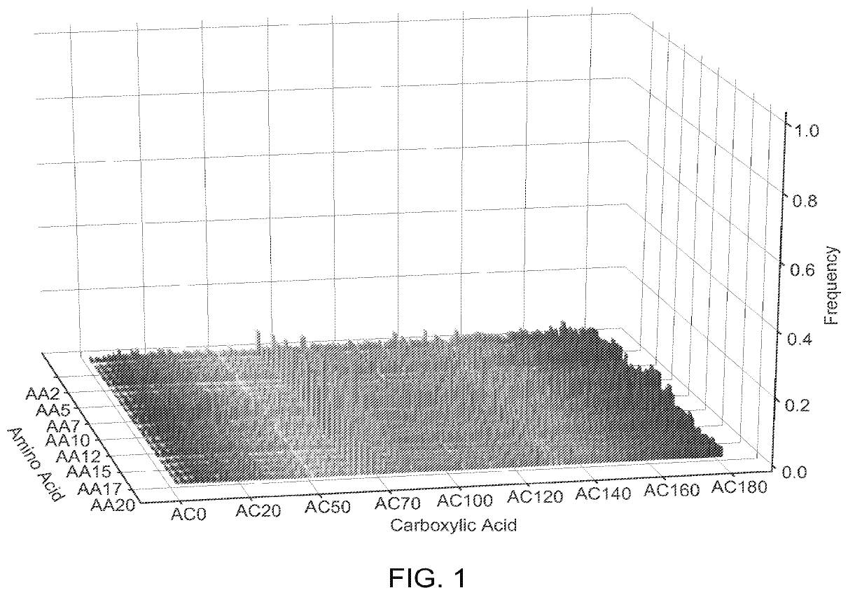

For each screening, a no target screening was concurrently performed that served as the negative control. This enabled the discardment of all the compounds in the library that bound non-specifically to the nickel beads. All the screening experiments were performed at 4° C. to retain SIRT3 activity. During the selection procedure, special care was taken to wash the beads post incubation of the protein and the library to get rid of non-specific binders as much as possible. Only one round of selection was performed, and final eluates were amplified, clustered and sequenced by Illumina iseq100. Prior to selection, we sequenced the native library to ensure that all the compounds are present at nearly similar concentrations so that the selection is not biased against any compound which might be present at a higher concentration than others. FIG. 1 shows the 3D plot of the native library showing the reads of the DNA barcodes corresponding to each compound. This is representative of the distribution of the compounds in the library. We observed that all the 194 derivatives of each of the 20 amino acids showed a similar distribution in terms of their frequency post next generation sequencing. Only AA5 derivatives appeared to be slightly overrepresented in the native library. The initial frequencies of the compounds helped us normalize the final output to cancel out any bias that might occur due to difference in the concentrations of the individual compounds in the native library.

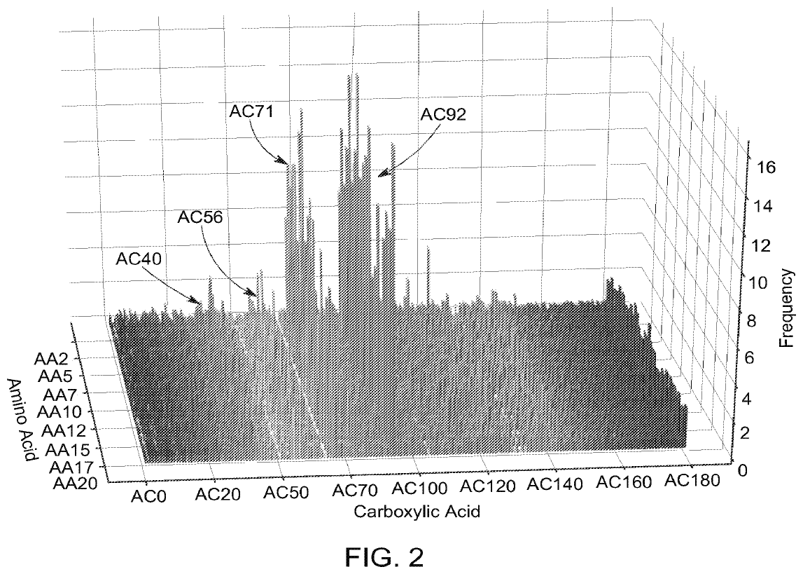

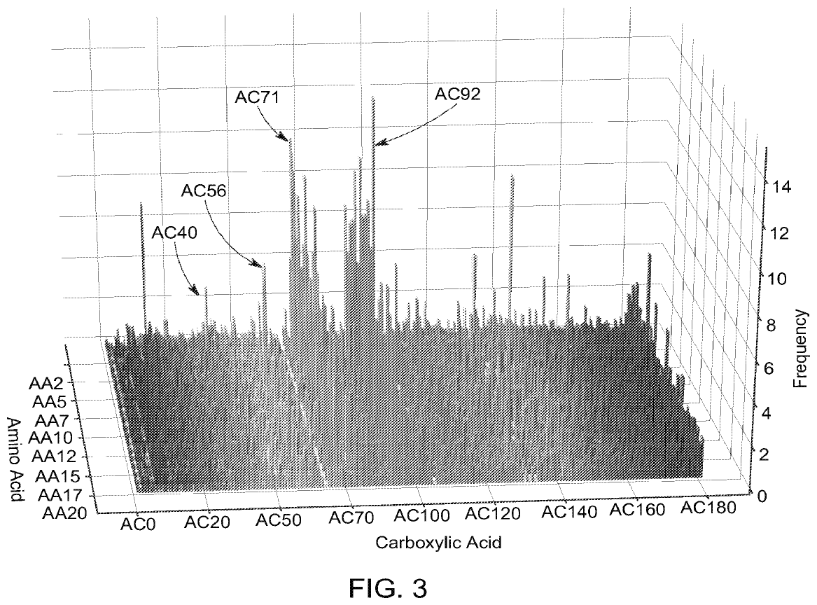

FIGS. 2 and 3 shows the 3D plot of the enrichment of the compounds over the background (no target selection) calculated after normalizing their abundance with respect to the total number of DNA sequence reads obtained for each selection. The top 10 highly enriched samples were selected for characterization, represented in Table 1. All these compounds showed an enrichment between 12-16 folds over the no target control selection.

Interestingly, for both the selection conditions, in the presence of Carba-NAD and OAADPr the same derivatives of carboxylic acids, AC71 and AC92 showed the highest enrichment. Among others, few derivatives of AC40 and AC56 showed significant enrichment between 4 and 8 folds over negative selection and would be worth investigating. The compounds were not overrepresented in the native library further corroborating the hypothesis that enrichment was observed only due to binding to SIRT3. These compounds showed similar enrichment when performed in duplicates for both the selection conditions. This emphasizes the role of both the AC71 and AC92 derivatives as putative binding partners of SIRT3, which is our rationale behind choosing them for further analysis. We observed that nearly all the compounds in the library did not show any affinity to the nickel beads indicating the veracity of the choice of the immobilization matrix. All the top 10 hits are derivatives of either AC71 or AC92 and in their corresponding amino acids four of them namely AA5, AA8, AA11 and AA12 are common in both the derivatives.

Thus, it was decided to synthesize 7 compounds of which 6 (AA12AC92, AA12AC78, AA12AC71, AA8AC92, AA8AC71, AA8AC40) were among the compounds that showed the best enrichments (top 10 hits) and one compound (AA12AC02Bis) that showed no enrichment, serving as a negative control. These compounds were synthesized off-DNA (The DNA chain has been replaced by a butyl group) in order to validate their binding affinity by Switchsense® and to test their modulation effect of the SIRT3 activity.

TABLE 1
The chemical structures of the top 10 hits from both the SIRT3 selection experiments in
the presence of Carba-NAD or OAADPr. These compounds were resynthesized without the
oligonucleotide conjugate for further characterization.

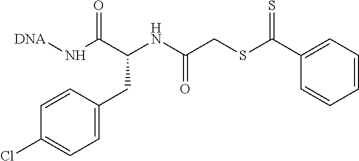

AA12-AC71

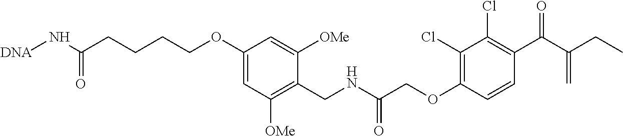

AA11-AC92

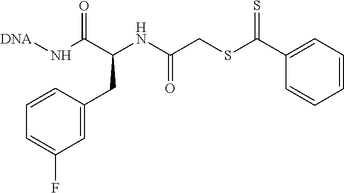

AA5-AC71

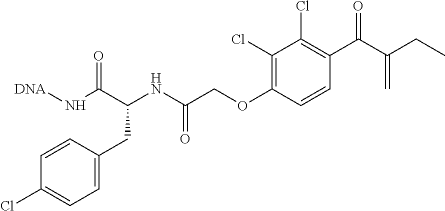

AA12-AC92

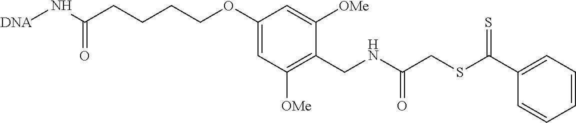

AA11-AC71

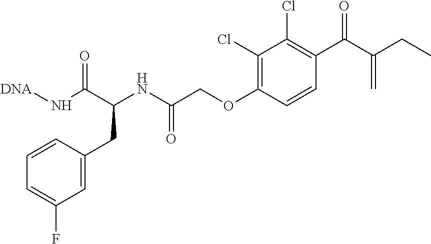

AA5-AC92

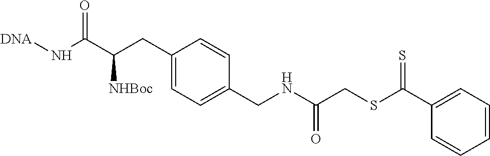

AA7-AC71

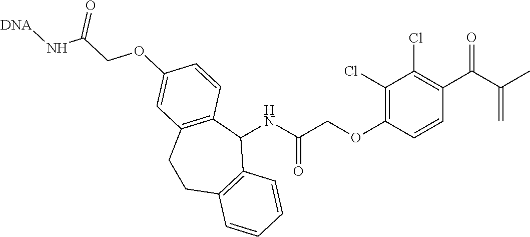

AA8-AC92

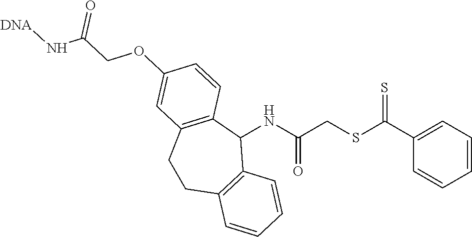

AA8-AC71

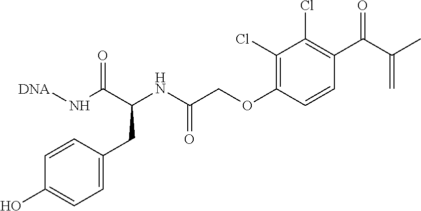

AA20-AC92

### SIR T3 Modulation Effect of Hit Compounds

The test compound is expected to experience three phases (presteady state, steady-state, and post steady state) during the enzymatic reaction. The pre and post-steady state can be considered as non-steady state (Conditions II for activity tests), in which the enzyme concentration vs. NAD⁺ substrate concentration ratio is high. Depending on the initial ratio of enzyme to limiting substrate in the system (and the ratio of enzyme to test compound concentration), the net effect of test compound over the course of the reaction can be either inhibitory or activating, with activation occurring if this ratio is above a threshold.

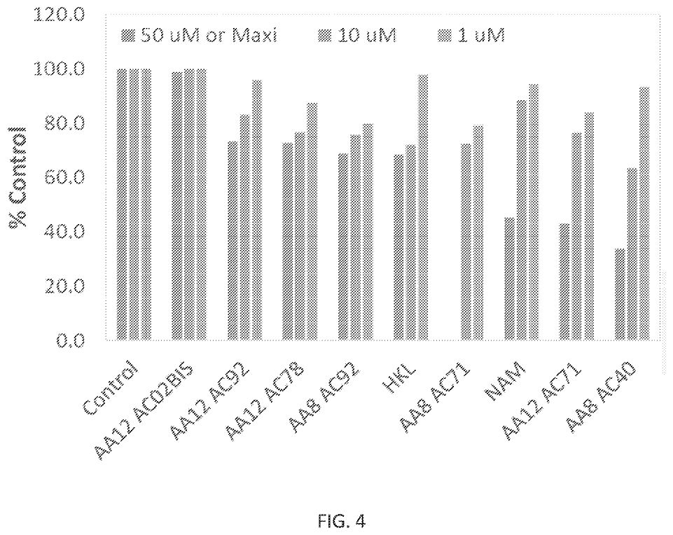

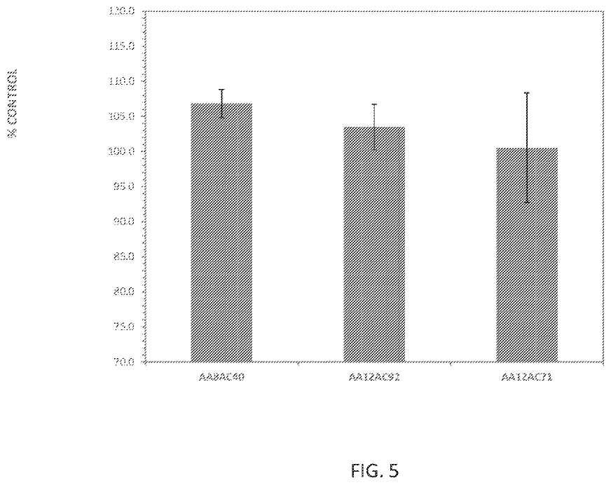

The 6 top ranked compounds from DEL screening were tested modulation effect of SIRT3 using fluorescence-based assay (FIG. 4). The selected compounds show different degree of inhibition effect under steady-state condition (Conditions I for activity tests). It was found that the SIRT3 deacetylation activity was inhibited 66.3% in the presence of 50 μM compound AA8AC40. It was also found that the inhibition level increases as the compound dose increasing. The IC₅₀ values for the compounds are expected to fall in μM range (Table 2). The negative control (AA12AC02BIS) has no contribution of SIRT3 modulation. The activity results agree with the DEL screening output.

TABLE 2
The potency of the selected compounds
on SIRT3 deacetylation activity.

| | 50 μM or Maxi | | | |
|---|---|---|---|---|
| | [ ], μM | % | [ ] = 10 μM | [ ] = 1 μM |
| AA12 AC02BIS | 50 | 99.0 | 100.0 | 100.0 |
| AA12 AC92 | 20 | 73.3 | 83.1 | 95.7 |
| AA12 AC78 | 25 | 72.7 | 76.6 | 87.5 |
| AA8 AC92 | 25 | 68.9 | 75.7 | 79.9 |
| HKL | 50 | 68.5 | 72.0 | 98.0 |
| AA8 AC71 | 10 | — | 72.4 | 79.1 |
| NAM | 50 | 45.2 | 88.5 | 94.3 |
| AA12 AC71 | 20 | 43.1 | 76.5 | 84.0 |
| AA8 AC40 | 50 | 33.7 | 63.5 | 93.3 |

### Nonsteady State Activation of SIRT3

The deacetylation activity of SIRT3 was detected in the presence of 10 μM compound of AA8AC40 under non-steady state condition (FIG. 5) using HPLC-based assay. In previous work, HKL was studied as a model non-steady state SIRT3 activator extensively. The test compound is expected to experience three phases (presteady state, steady-state, and post steady state) during the enzymatic reaction. The pre and post-steady state can be considered as non-steady state, in which the enzyme concentration vs. NAD⁺ substrate concentration ratio is high. Depending on the initial ratio of enzyme to limiting substrate in the system (and the ratio of enzyme to test compound concentration), the net effect of test compound over the course of the reaction can be either inhibitory or activating, with activation occurring if this ratio is above a threshold value.

In the current study, the top hit compounds have bigger volume structurally comparing to HKL (structures below). The computational study indicates that AA8AC40 binds to SIRT3 externally.

### Protein-Compound Docking

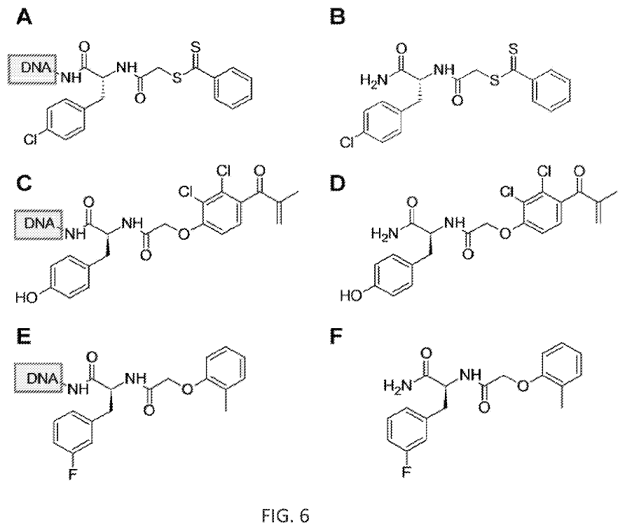

Ligand docking, as described in Materials and Methods, was performed on top-binding ligands from the DEL, as identified by the affinity mediated selection assay. However, the large size and flexibility of each DNA-tagged and combinatorially-generated ligand made it impossible to dock the entire complex to SIRT3. To address this problem, we decided to only dock the structure of combinatorial ligands, specifically their C-terminal amidated form, to SIRT3. In addition to most active combinatorial ligands, we also choose a few inactive ligands, also identified by the same assay, as negative controls to validate the choice of binding site and docking procedure. Structures for some positive and negative controls used in our docking studies are shown in FIG. 6.

Despite the substantial size differences between DEL-derived ligands and compounds known to bind at internal sites in SIRT3, such as Ex-527 and Honokiol, it was decided to first explore known internal sites in that protein. However, attempts at docking both actives and inactive hits from the DEL-library to internal sites in two structures of SIRT3, 4BVG and 4FVT, were not successful. There results were not unexpected for two reasons, which are as follows: (1) there is no worthwhile overlap between pharmacophores or other structural features of hits obtained from DEL-library and known compounds capable of binding at internal sites in peptide and NAD⁺ (or Carba-NAD) bound structures of SIRT3 such as Ex-527 and Honokiol; (2) Hits, even without their DNA-tags, are noticeably longer than either Ex-527 or Honokiol and available space within internal sites to accommodate compounds, in the presence of peptide and Carba-NAD, is barely adequate to accommodate these two known ligands. While it is possible to accommodate larger compounds in that site, doing so requires the displacement of NAD⁺, or Carba-NAD, from it such as that is seen for some potent inhibitors of SIRT3.

Attempted docking of hits and control at internal site within both SIRT structures (4FVT and 4BVG) reveals a few more problems. Firstly, clustering of top poses for hits from active ligands is poor at internal sites within both structures, which is not helped by a significant percentage of them lying partially outside cavity (not shown). Furthermore, directionality of top docked poses for many hits is not compatible with location of DNA-tag linkage. Inactive control compounds from library also exhibit substantially better clustering than hits when docked to internal sites in both structures (not shown). Hence it is unlikely that hits from DEL-library bind at internal sites in SIRT3, leading us to the possibility that they bind to more exposed sites which are on (or near) the surface of protein.

A search was then initiated for putative binding sites in SIRT3 which were either atypical or external. Two such sites were readily identified in 5H4D and 4O8Z, one in each. Both were defined by a co-crystallized, though weak, ligand of SIRT known as Amiodarone. However, once again, the top docked poses of DEL-library derived hits did not display any significant overlay with each other when docked at the external site in either protein structure (not shown). Moreover, the top docked poses of positives had poor to non-existent hydrogen bond interactions with residues lining the site in 5H4D and 4O8Z.

Next, 4FVT and 4BVG were scanned, using a geometry-based method, for locating potential binding sites. We were able to identify one atypical binding site large enough to partially accommodate positive library hits (in addition to known internal site) in 4FVT. A similar scan of 4BVG revealed two more atypical potential binding sites large enough to accommodate these compounds. Attempts to dock positive hits to the atypical binding site in 4FVT revealed some degree of overlap between top docked poses for a few of the positive hits, however the degree of pharmacophore overlap for top poses of different ligands was still poor and no conserved pattern of interactions with receptor was seen.

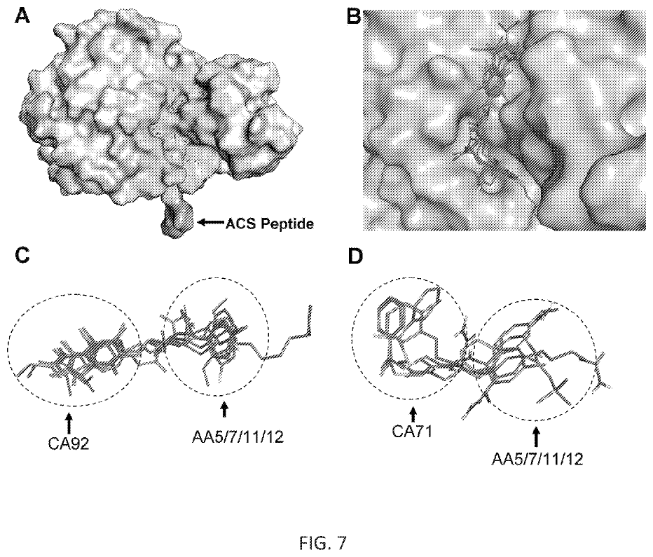

Docking at the first putative atypical binding site in 4BVG revealed a high degree of overlap for top docked poses of most positive hits. Only 2/10 positive hits (AA8-AC71, AA8-AC92) did not consistently overlap with top docked poses of the other 8/10 positive hits as shown in FIG. 7. Top poses of most positive hits also demonstrated a high degree of pharmacophore overlap and conserved interactions with residues lining the site. Notably inactive controls also dock well at this site but display fewer interactions (hydrophobic and hydrophilic) that hits with lining residues.

The second putative atypical site in 4BVG is similar to the one defined by Amiodarone in 5H4D and 4O8Z. The top poses of hits do display some pharmacophore overlay with each other (not shown), but the degree of such overlap nowhere near that seen at atypical Site 1 in 4BVG, and therefore the later is the most likely binding site for hits from DEL-library. It should, however, be noted that 2/10 tested hits did not dock properly at atypical Site 1. Also, developing ligands for atypical Site 1 in 4BVG might be harder than usual, since it is shallower and more exposed to solvent than is typical for small-molecule sites.

### II. Identification of Hits Capable of Mechanism-Based Activation of Sirtuin-3 Using Virtual Screening (Formulas III-XIV)

AWS cloud servers were used for virtual screening of a 1.2 million compound database from ChemBridge using AutoDock Vina. Two QM-MM geometry optimized SIRT3 complexes (4FVT and 4BVG) were used as receptors in this docking exercise. Hits were identified based on their docking scores (in AutoDock Vina and MOE) for these two receptors as well as their H-bond interactions with specific residues lining the binding site, including those in cofactor binding loop. Honokiol was included as a positive control in the library and subsequently identified as a hit compound according to these criteria. Top 20 compounds, out of the top thousand, were chosen based on their docking scores (between −11.7 to −9.5 as shown in Table 3). AutoDock Vina scores for selected compounds were significantly better than average values of whole dataset (median−5.5) indicating a good fit to the binding site, demonstrating that compounds with higher scores exhibited better overall interaction with SIRT3 than those with lower values. The residues having H-bond with compounds and the degree of receptor buried for each compound were also reported in Table 3.

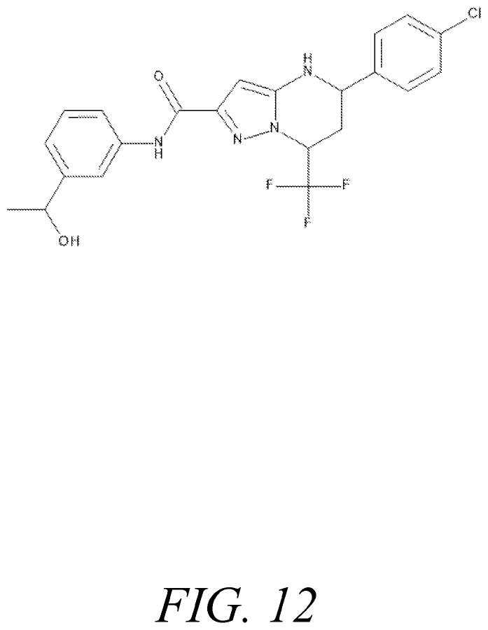

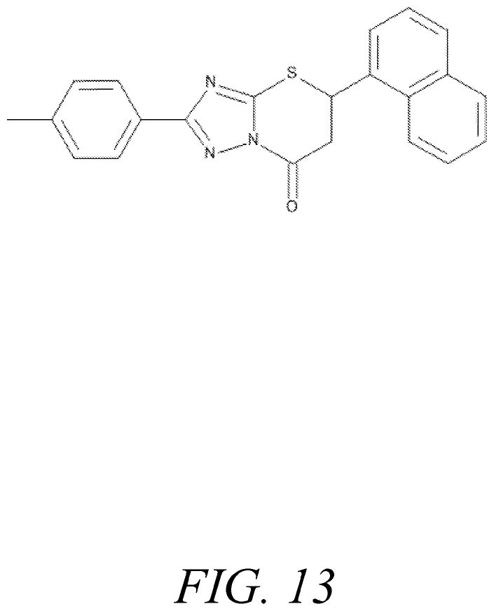

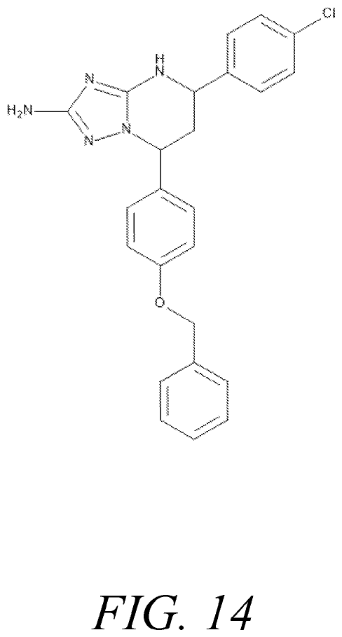

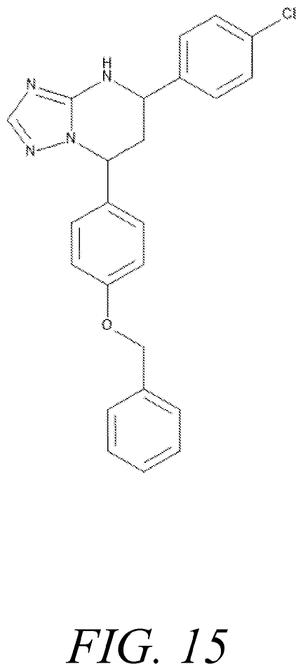

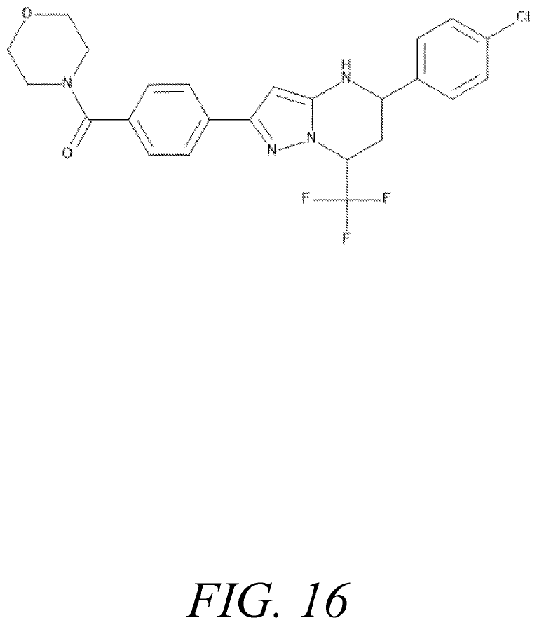

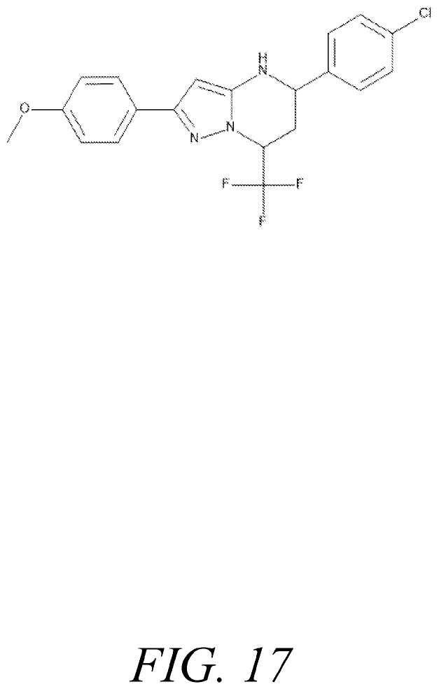

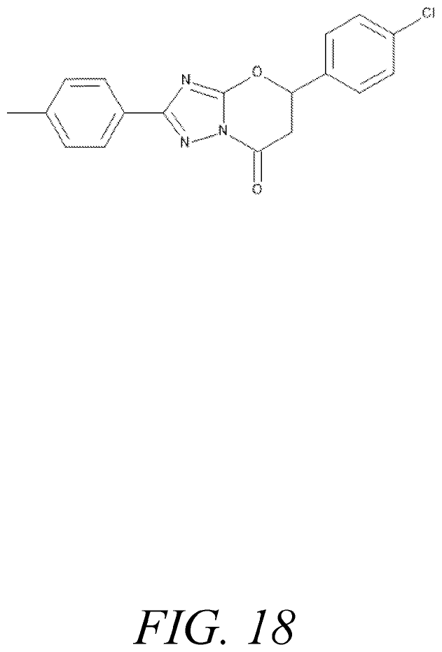

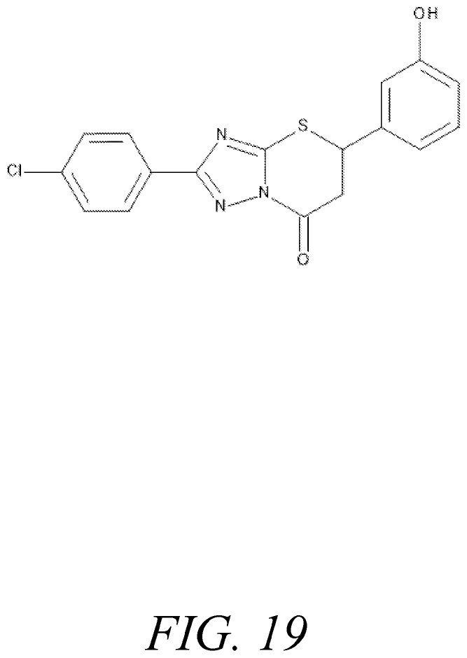

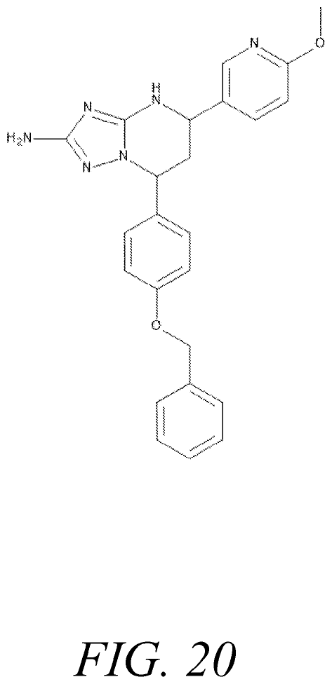

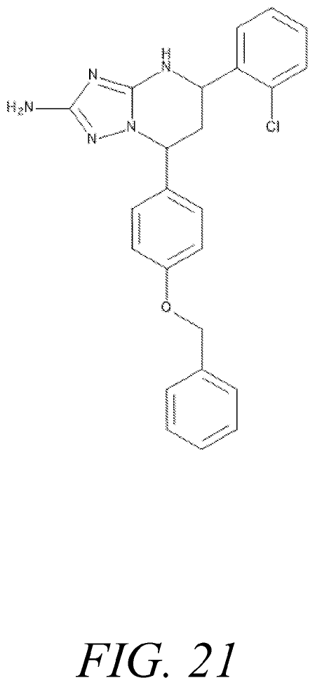

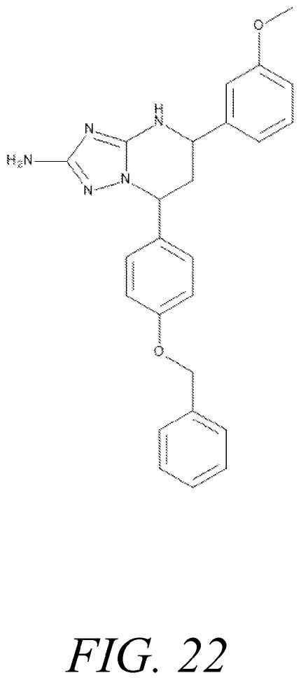

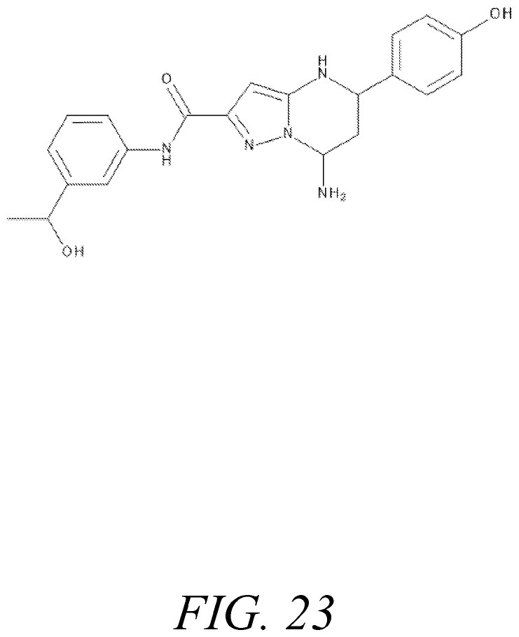

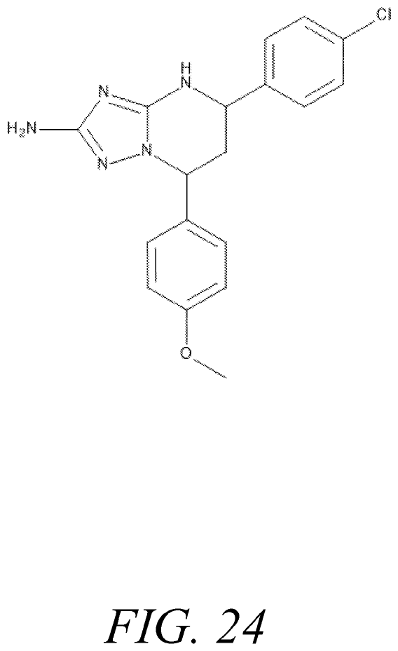

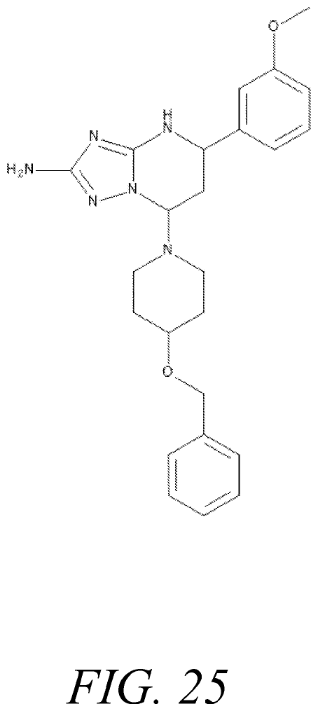

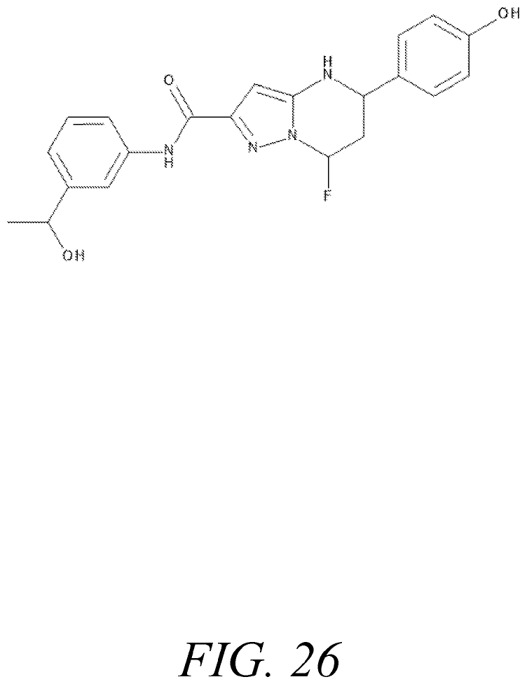

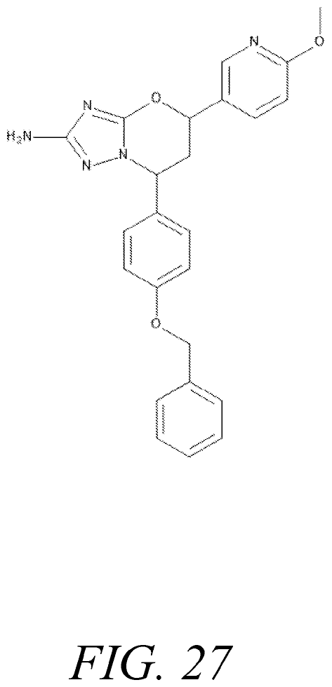

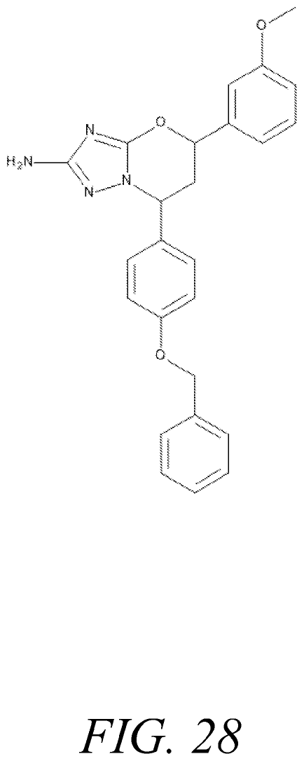

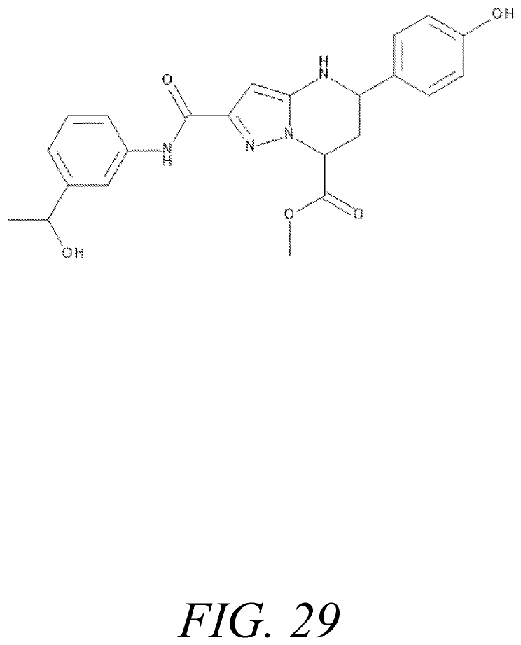

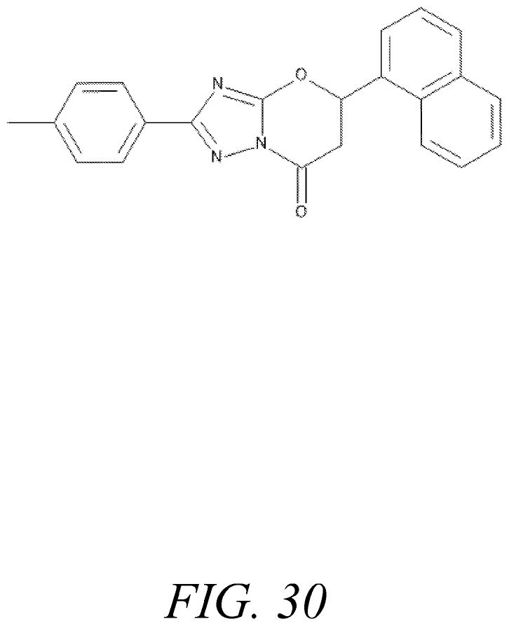

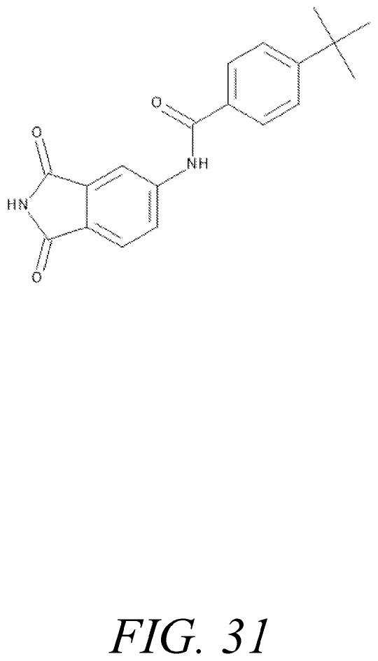

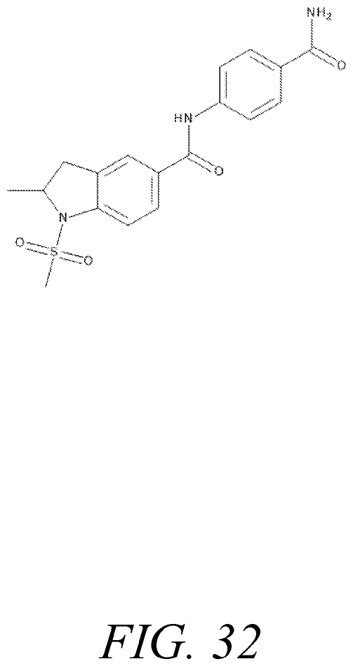

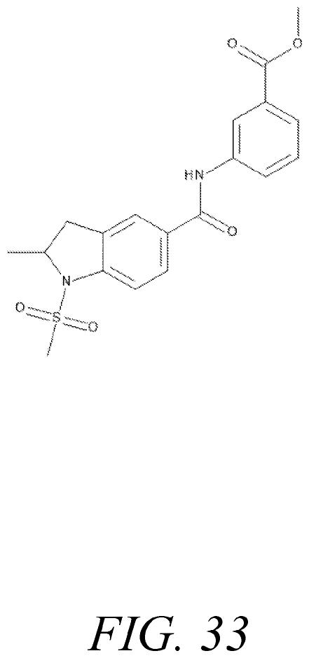

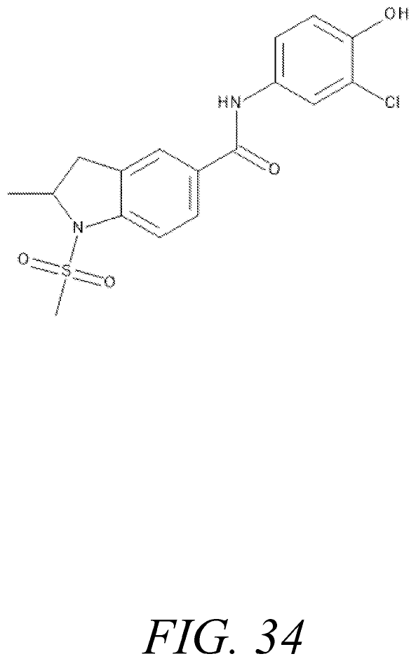

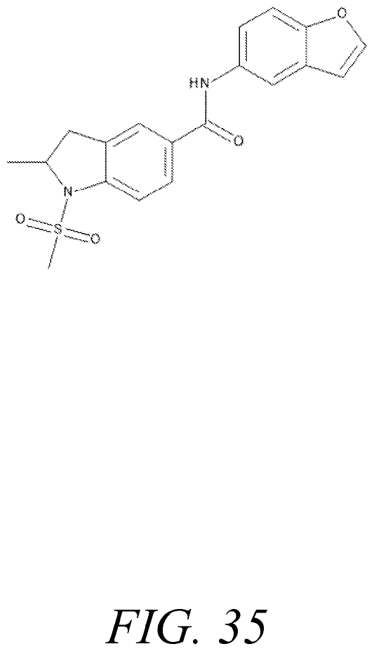

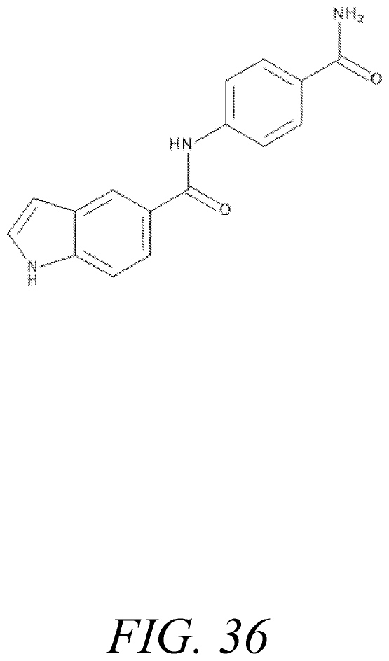

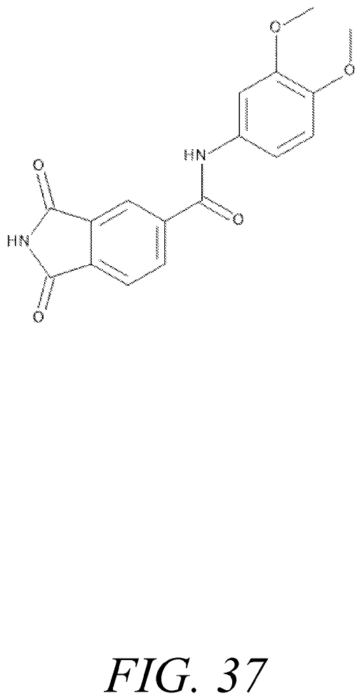

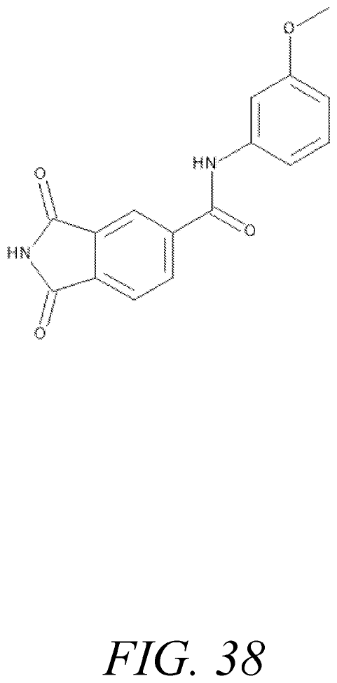

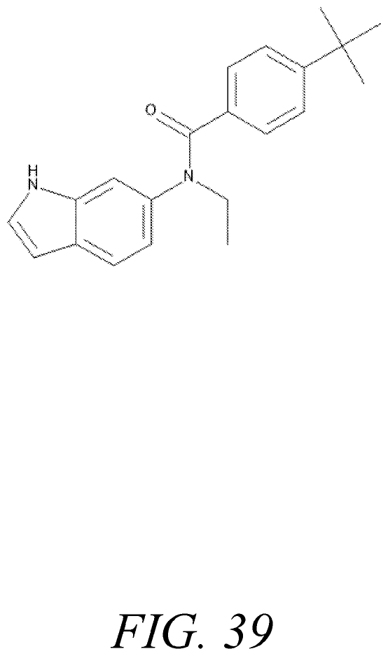

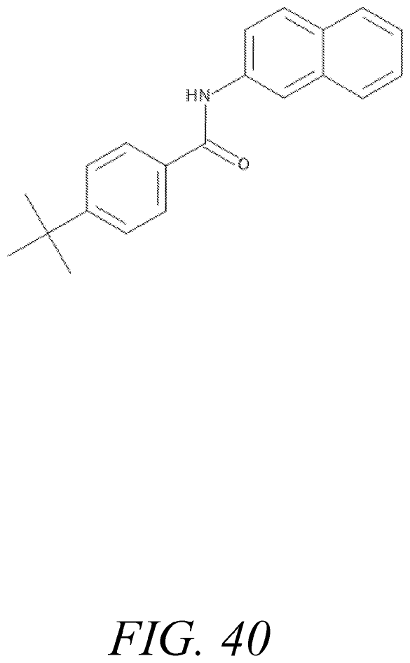

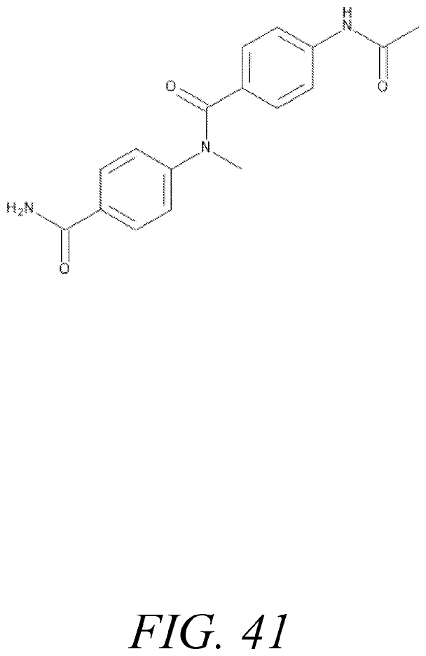

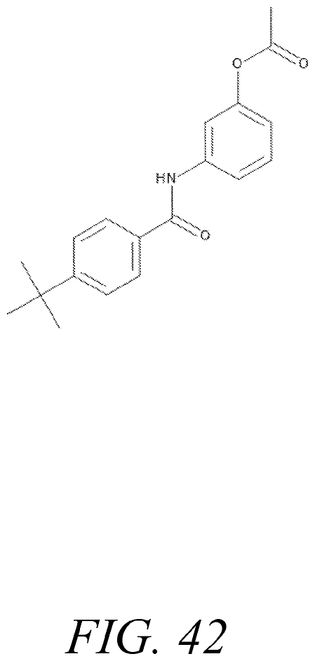

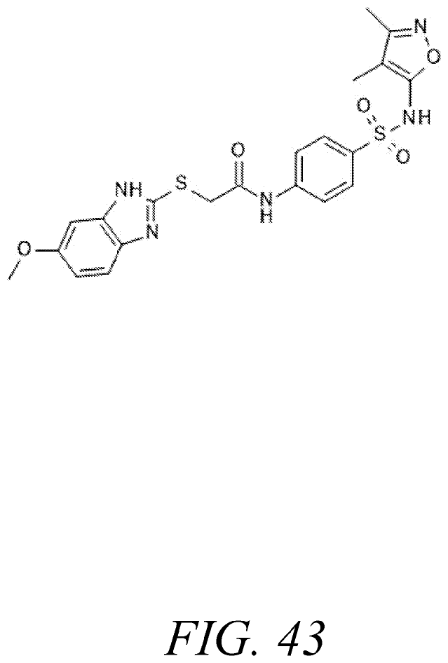

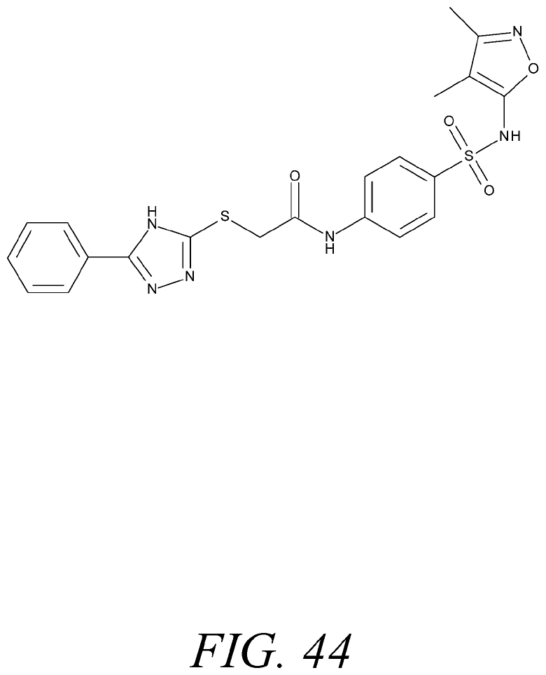

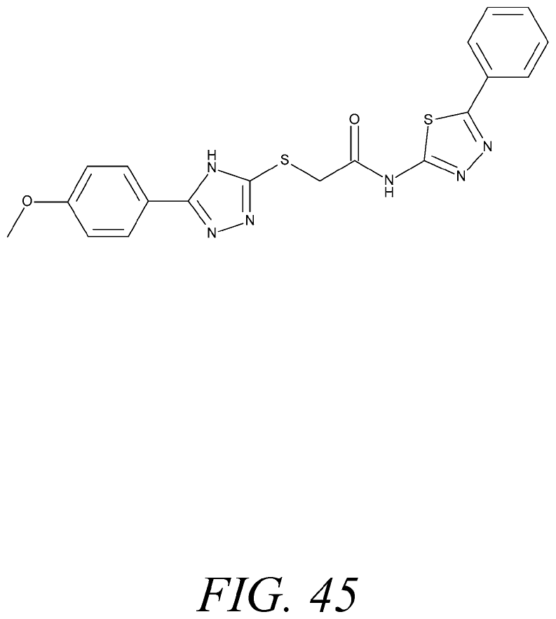

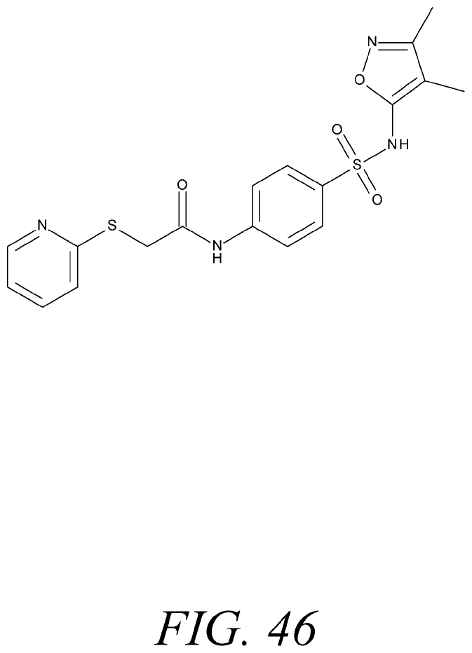

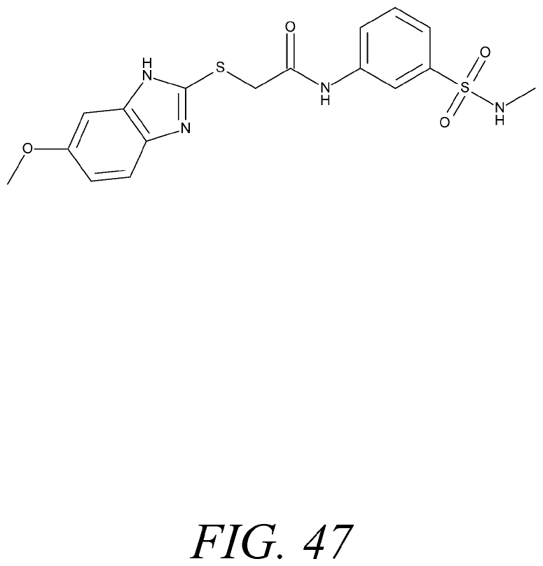

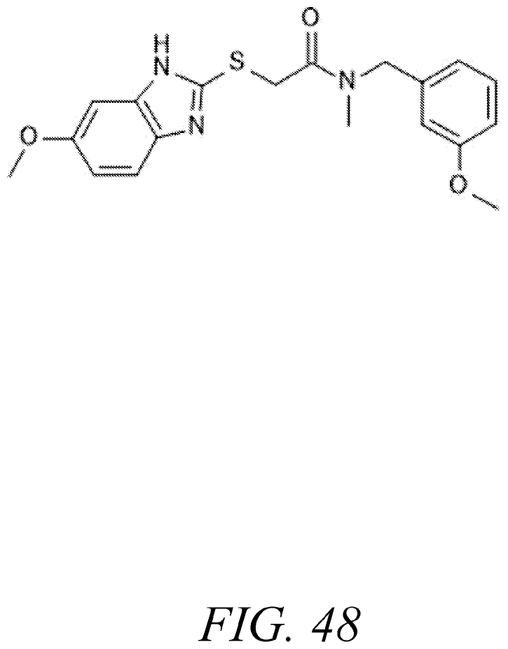

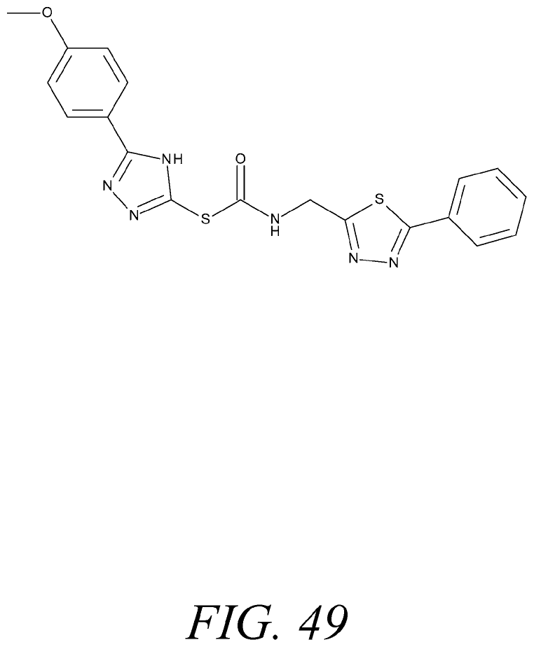

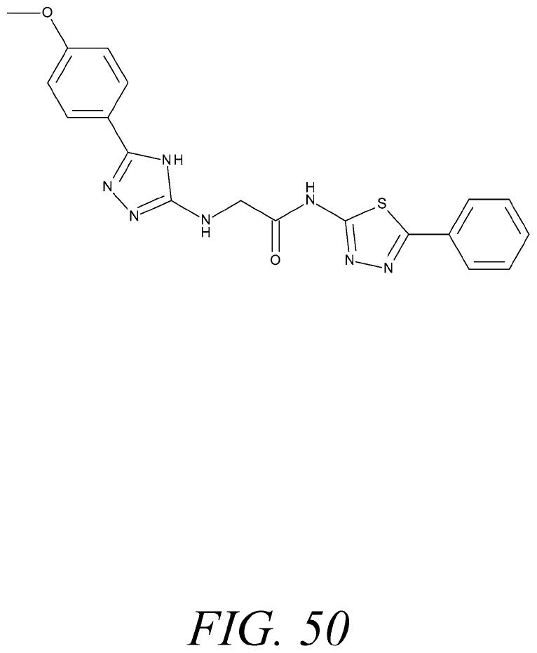

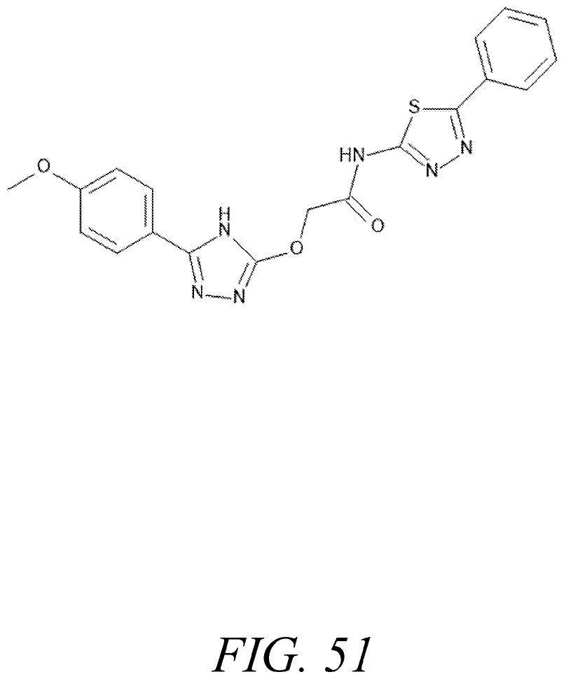

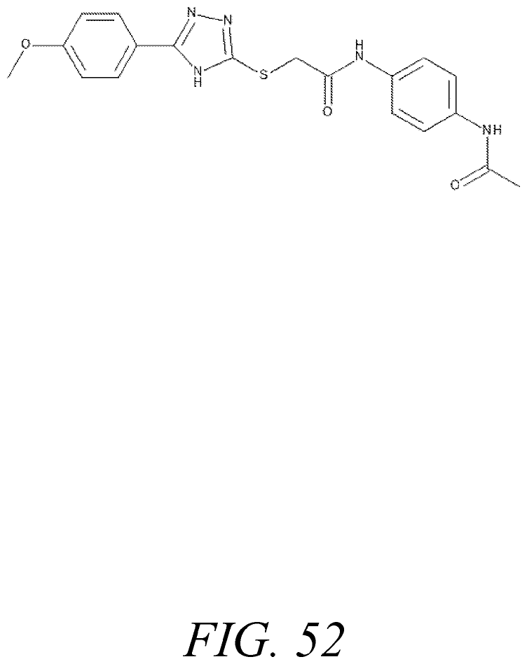

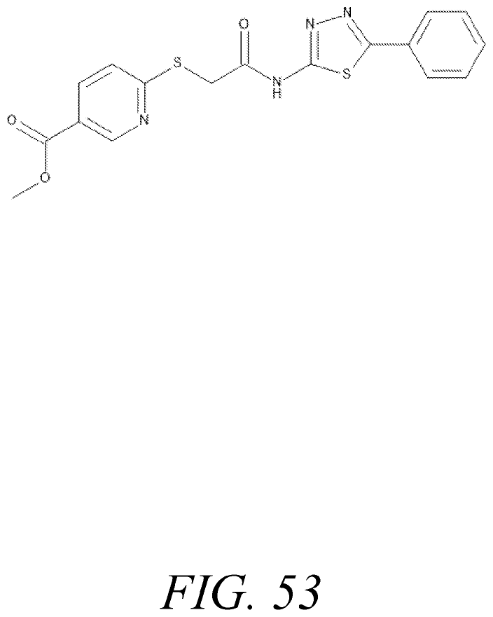

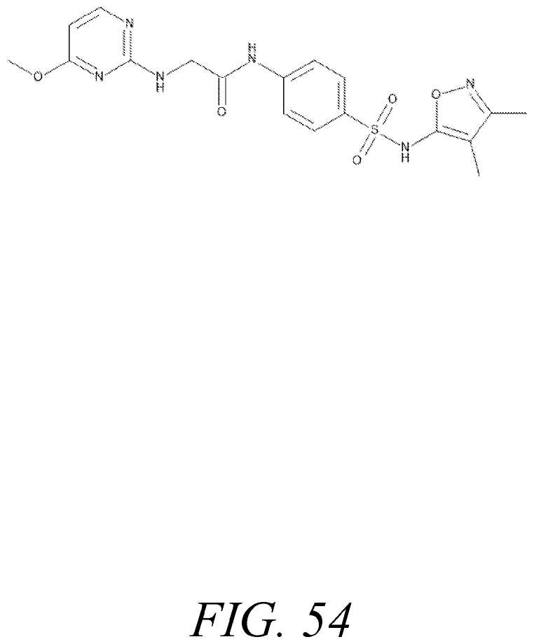

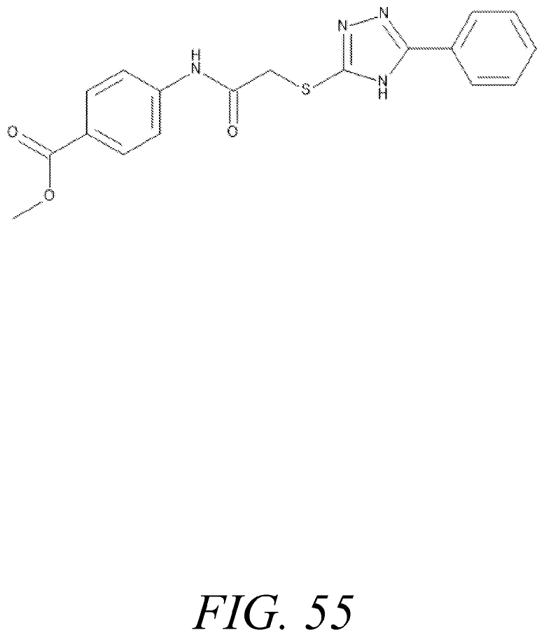

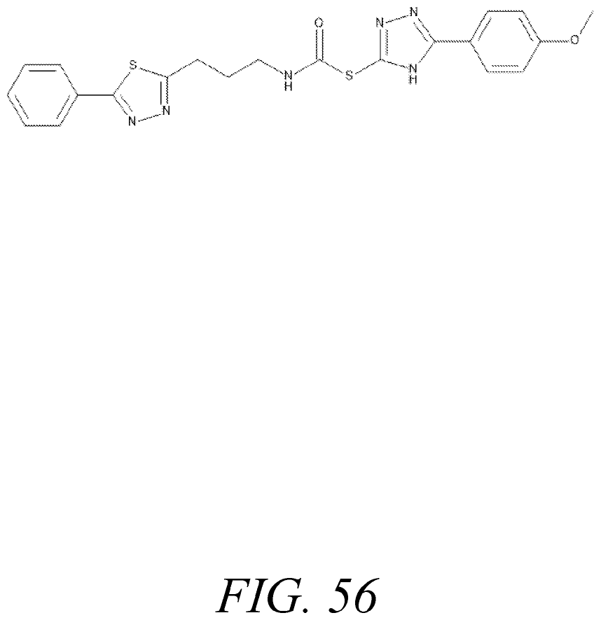

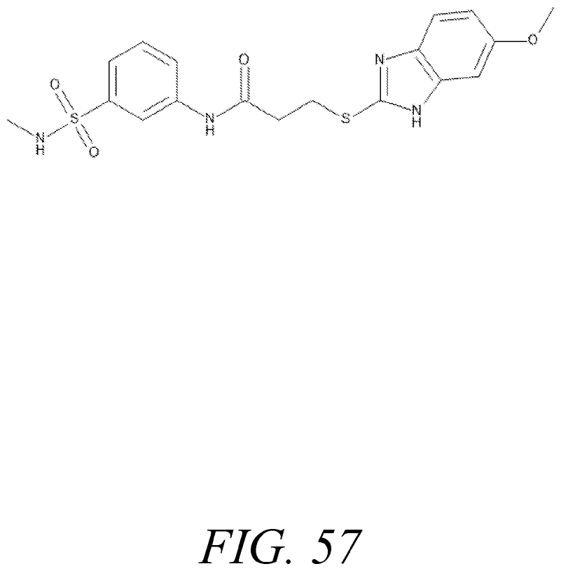

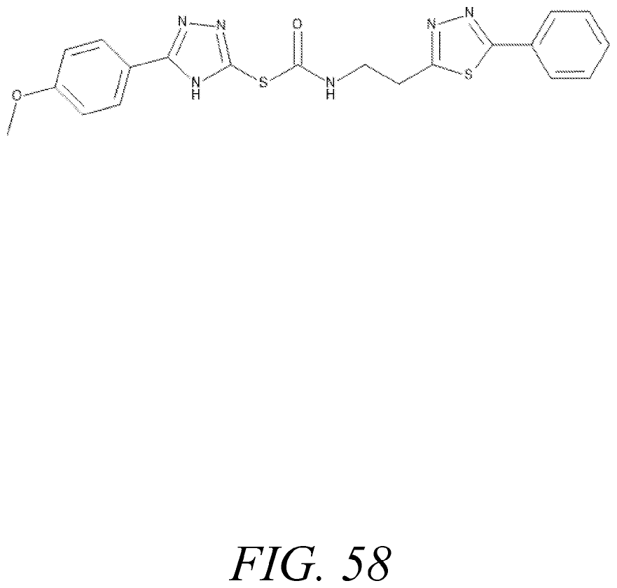

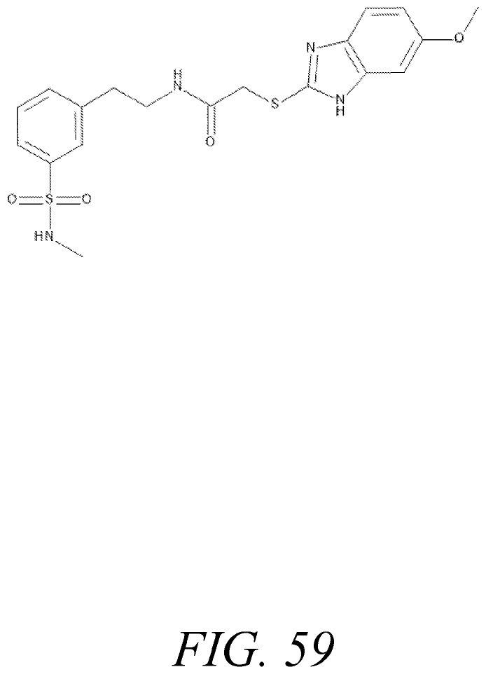

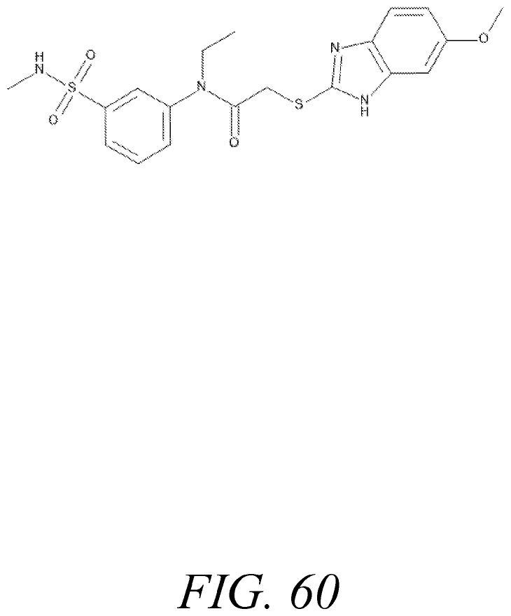

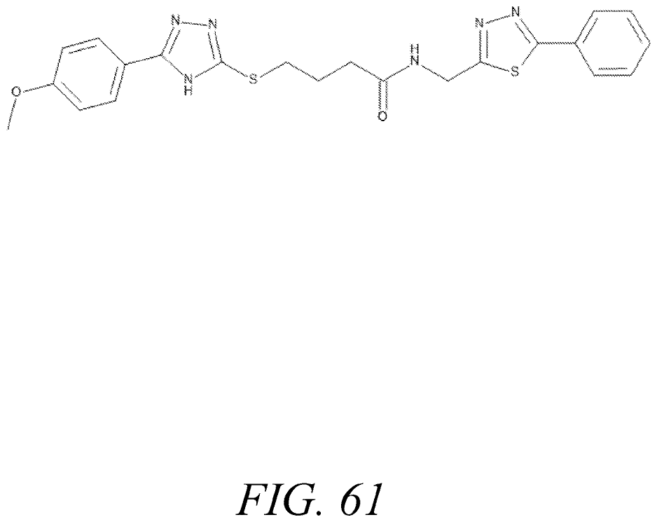

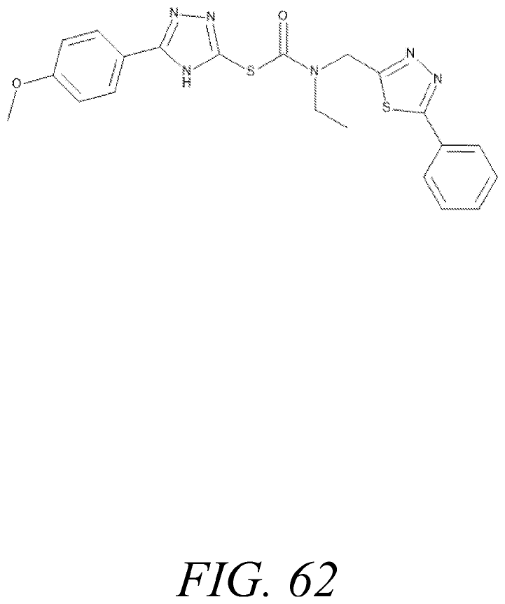

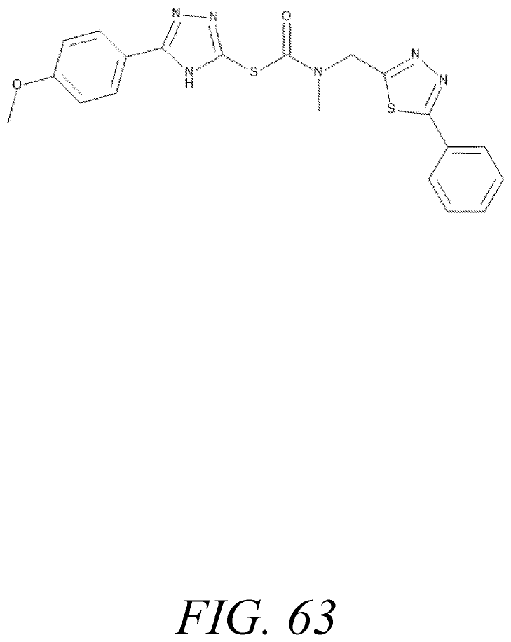

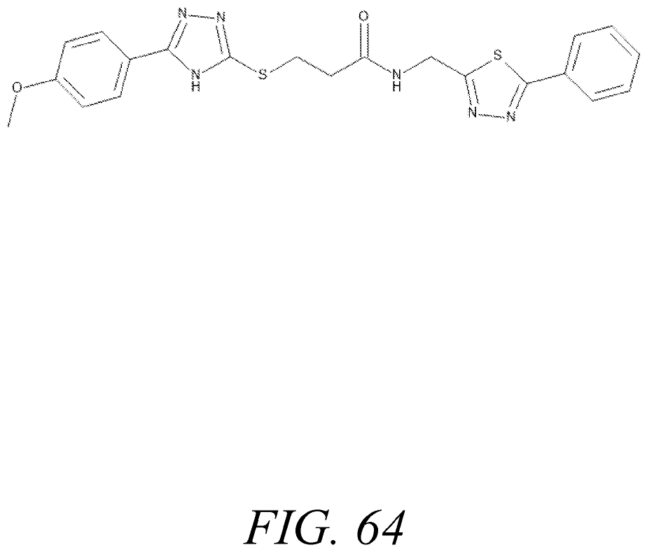

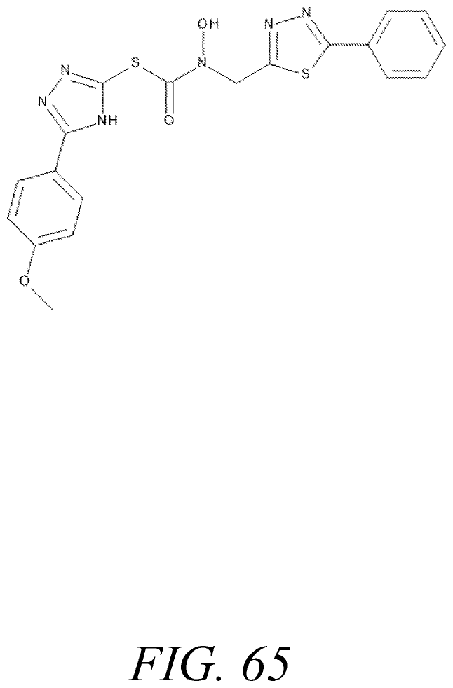

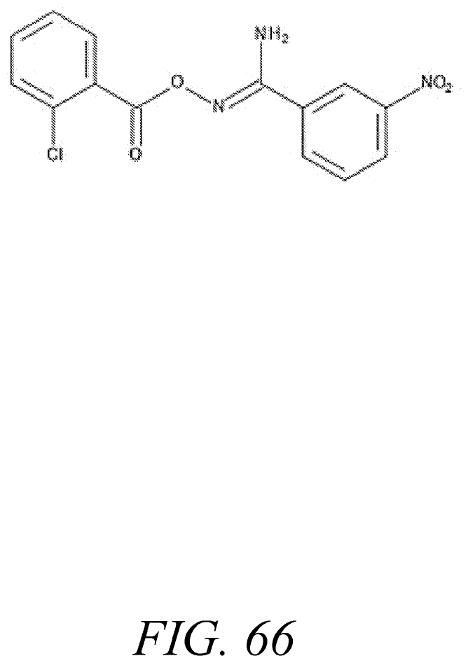

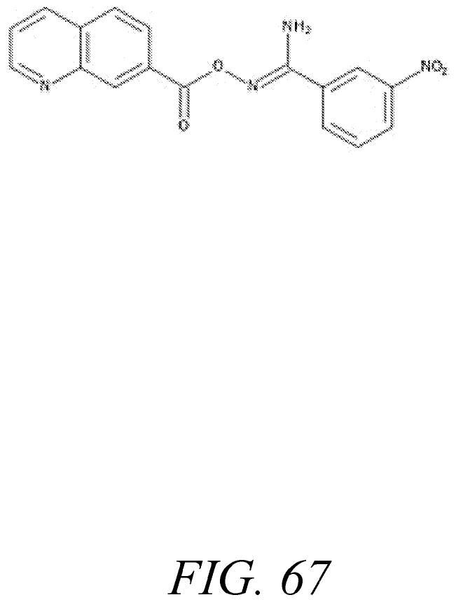

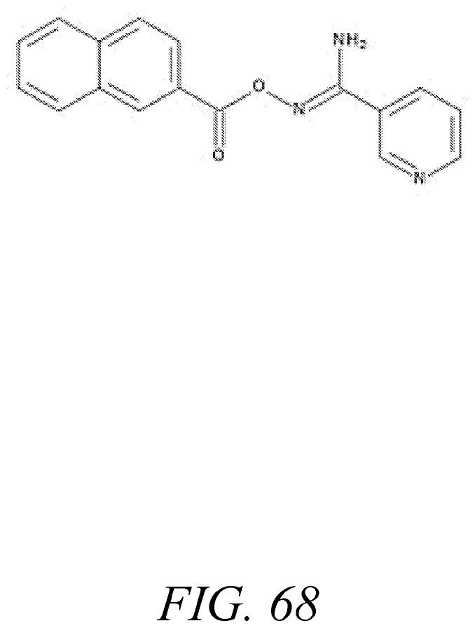

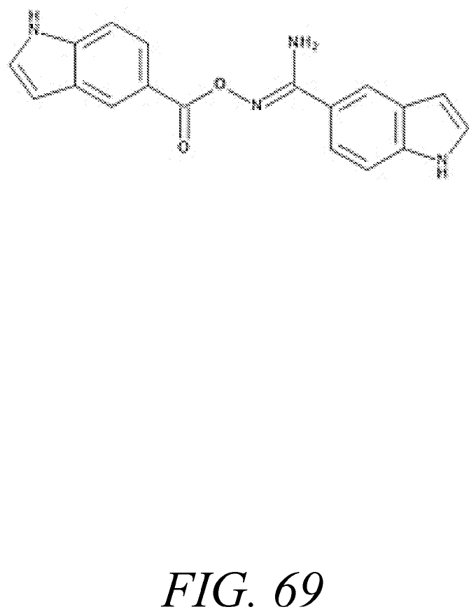

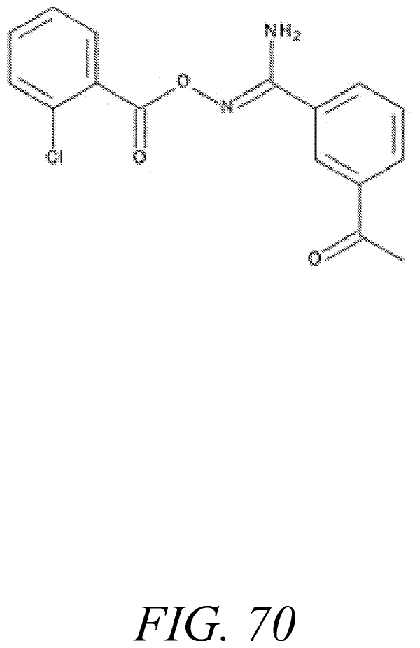

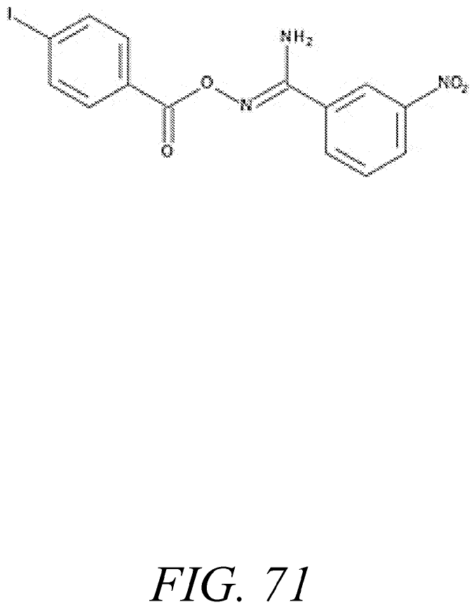

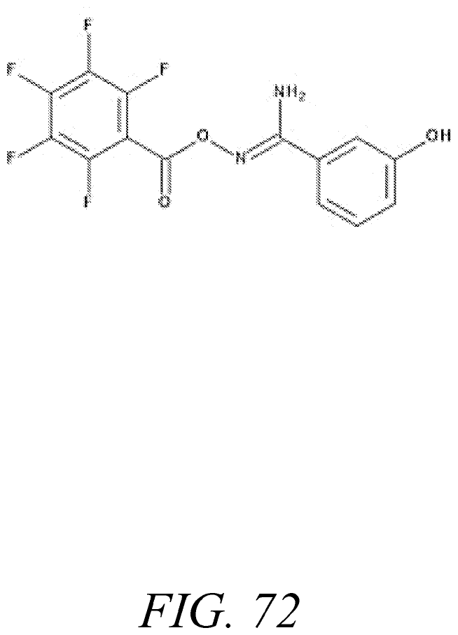

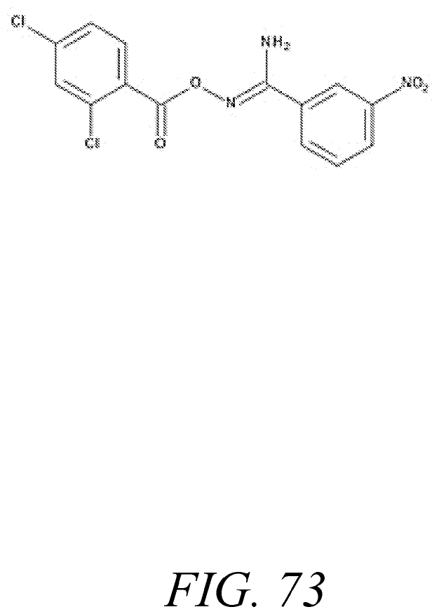

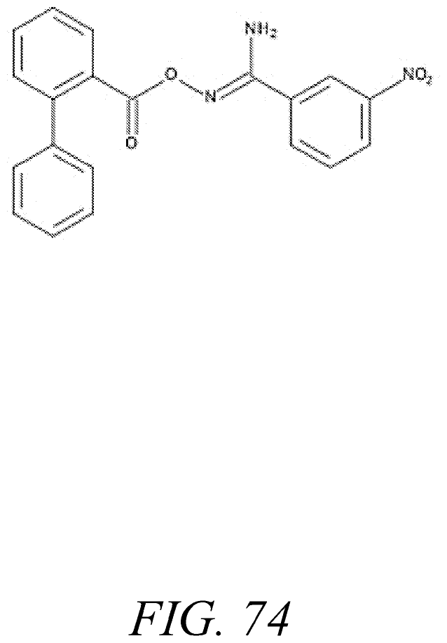

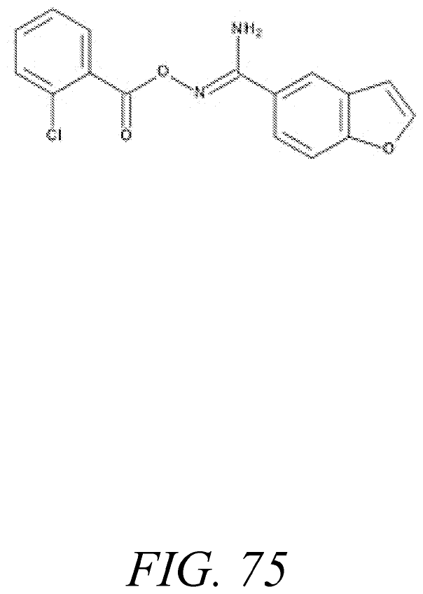

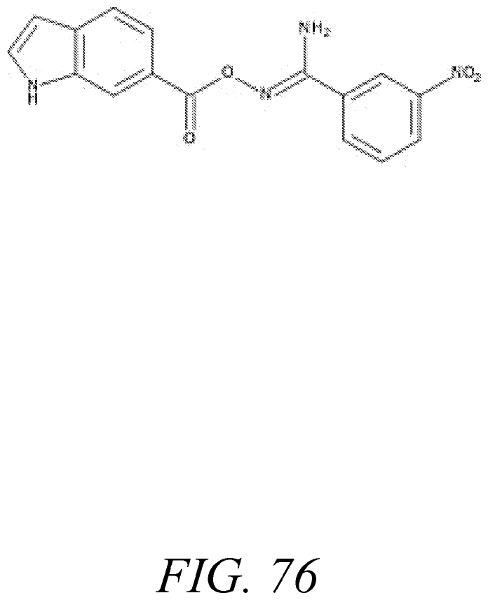

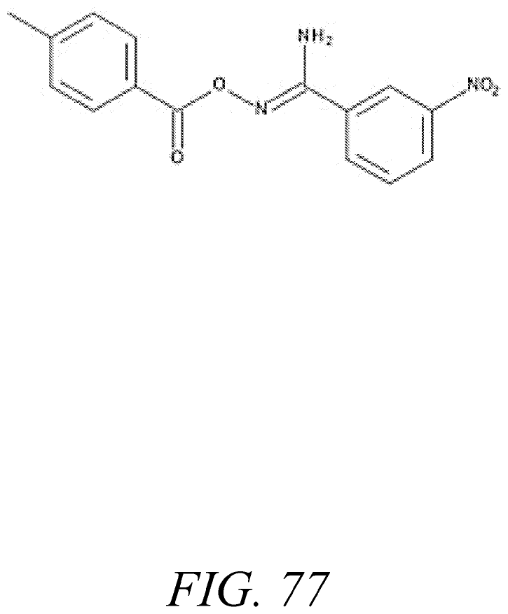

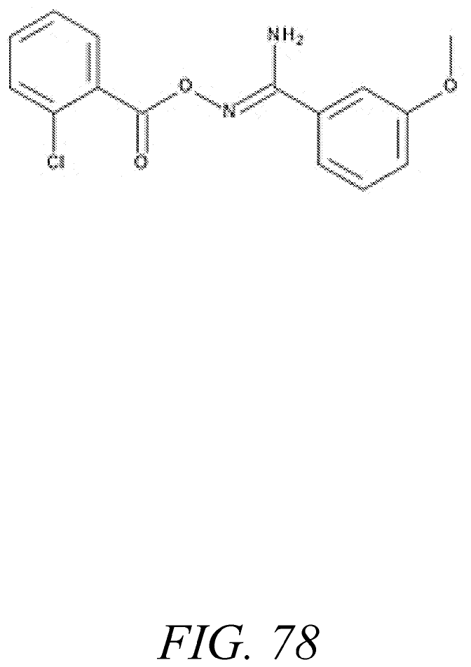

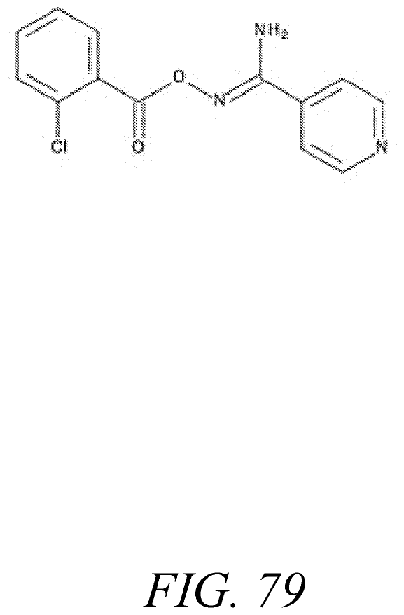

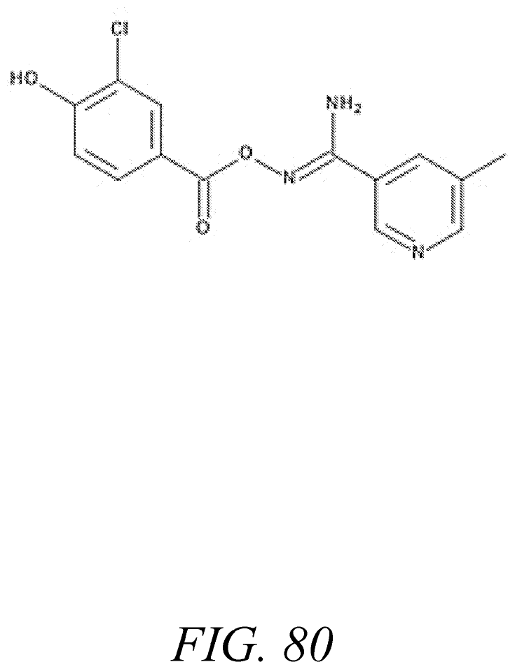

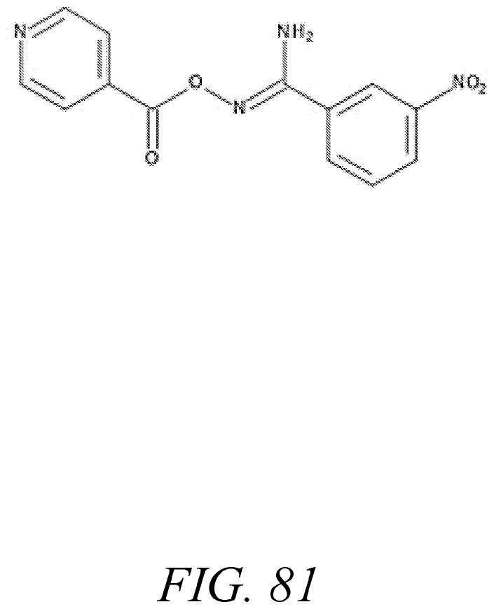

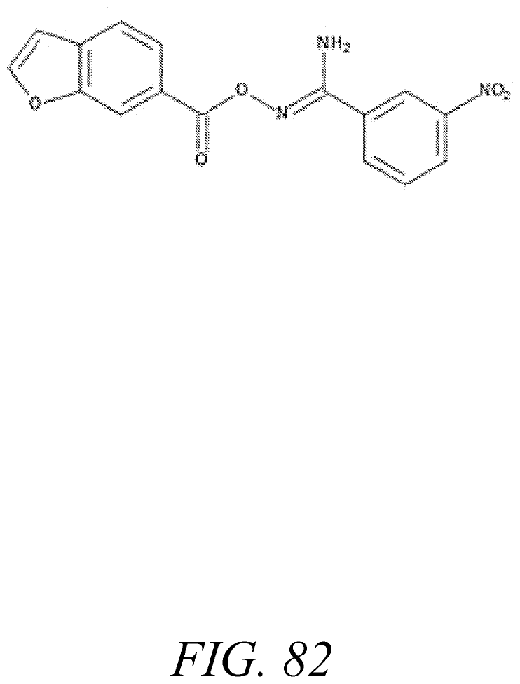

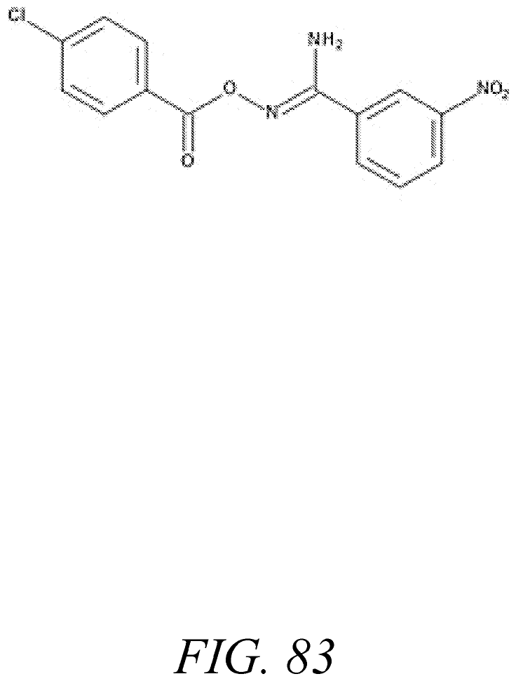

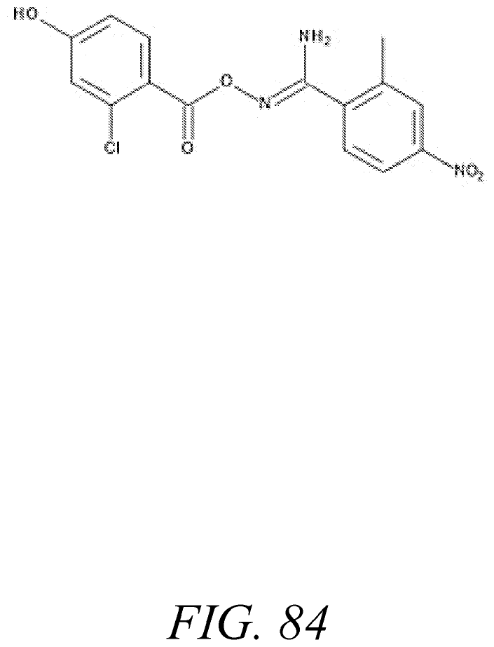

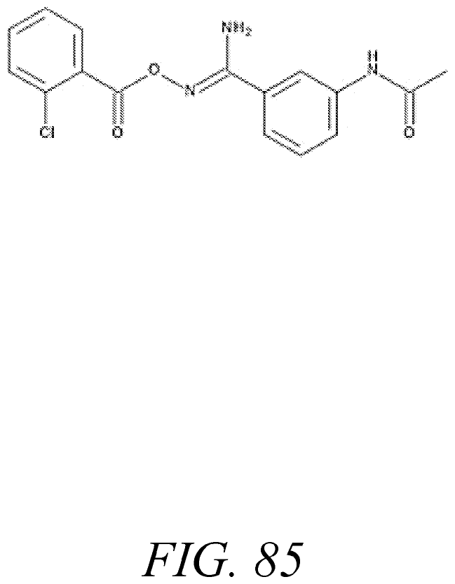

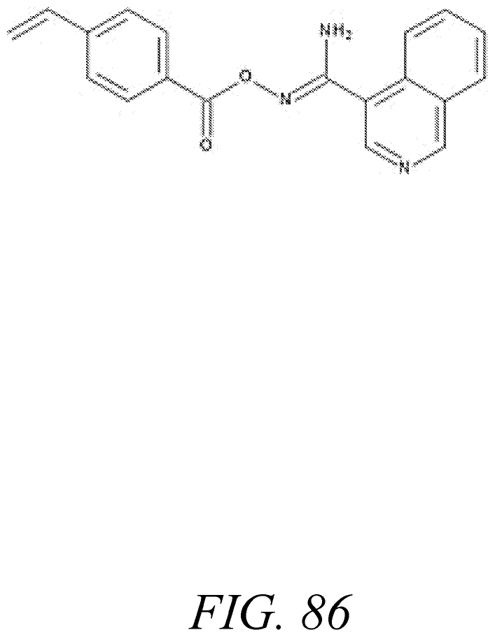

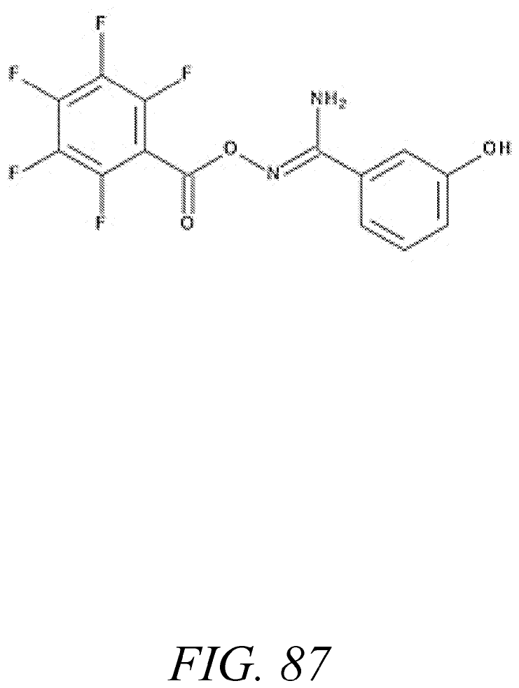

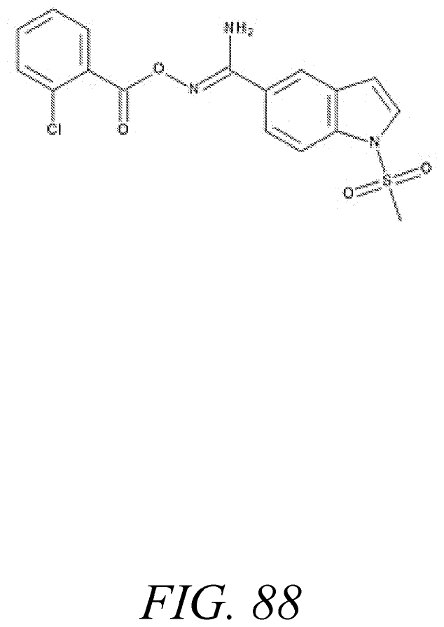

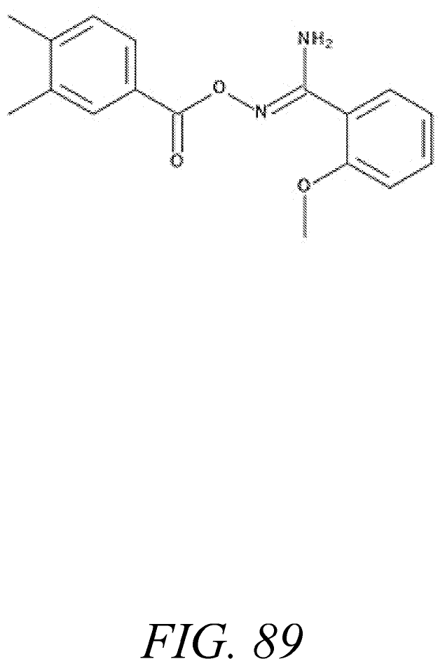

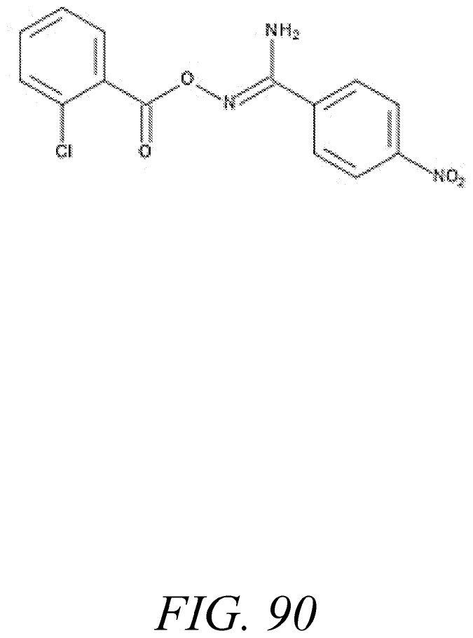

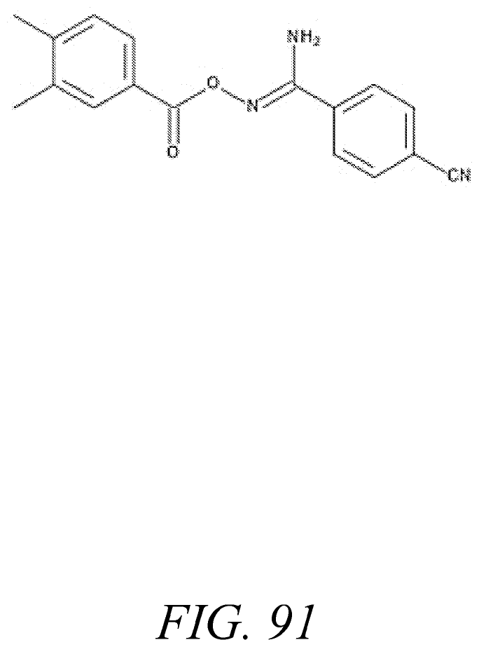

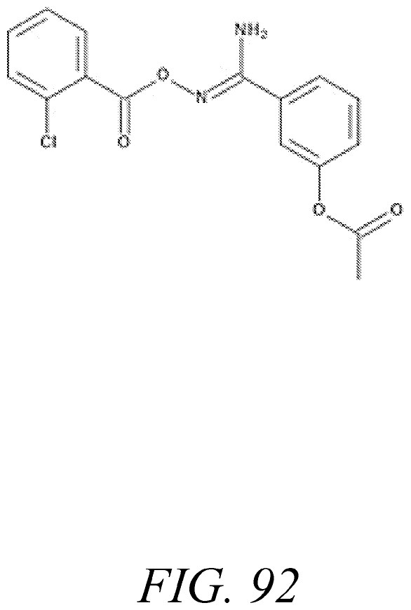

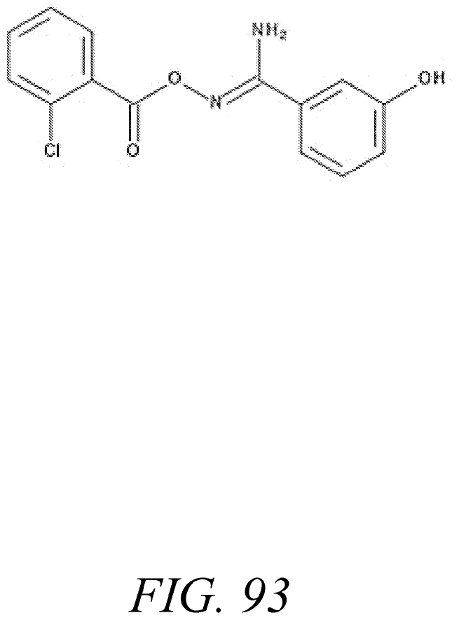

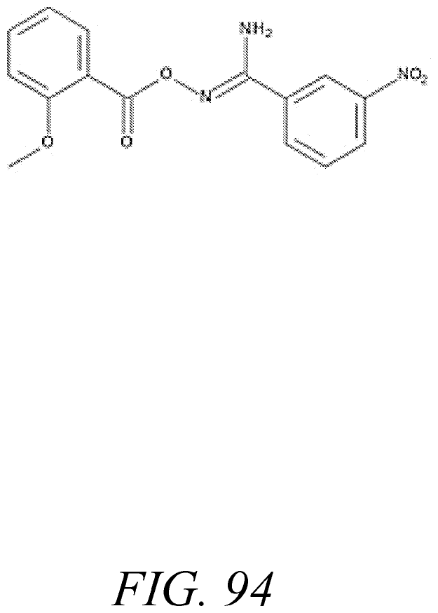

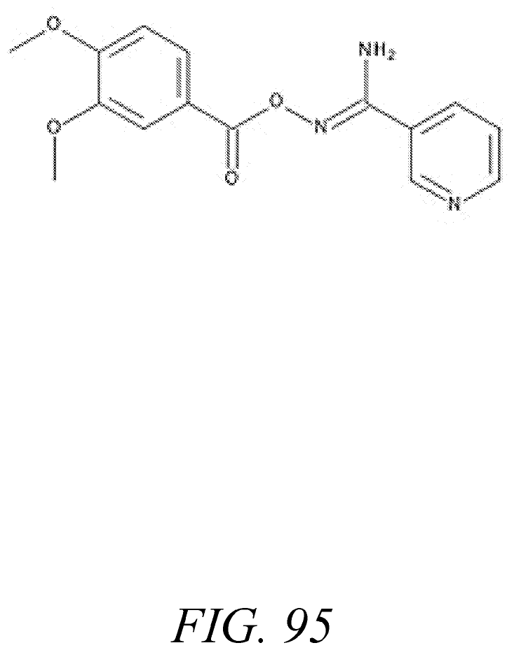

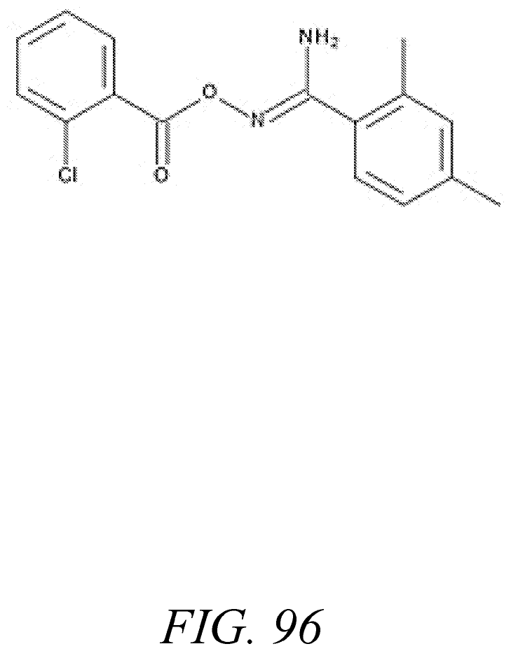

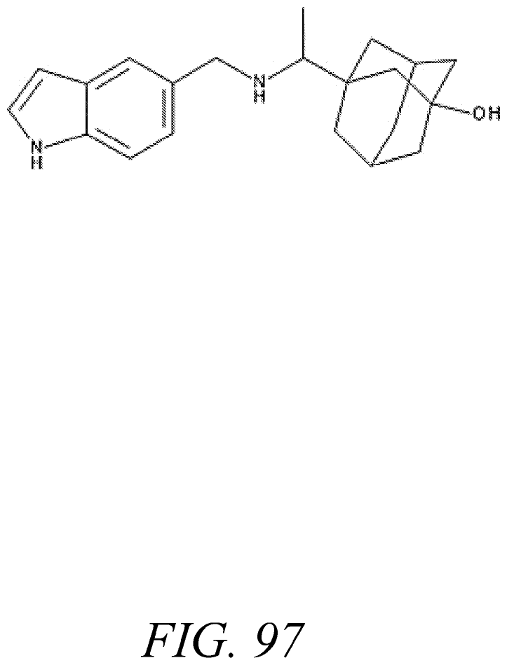

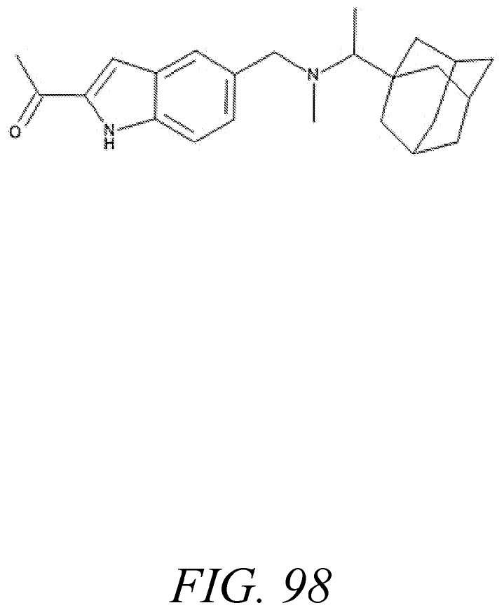

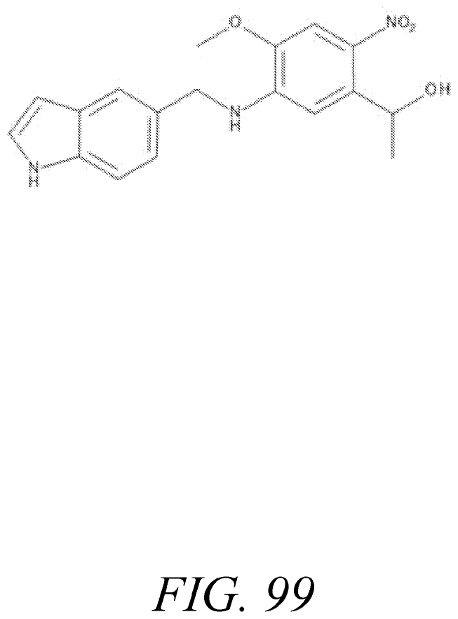

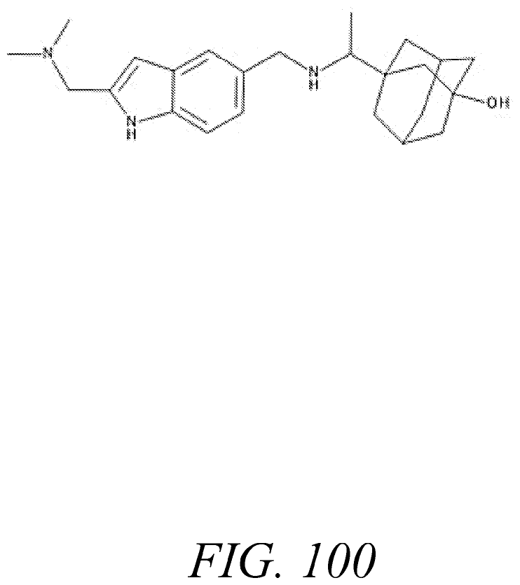

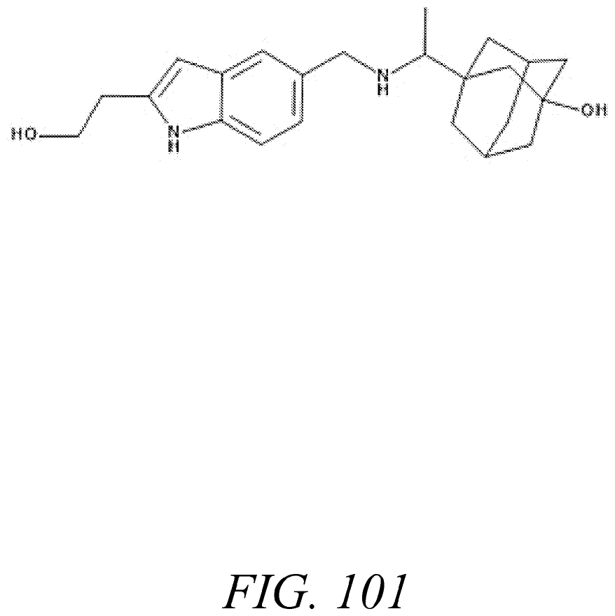

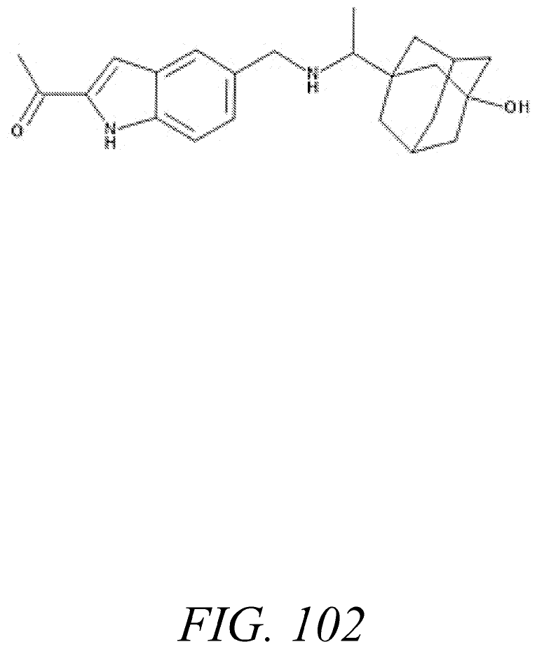

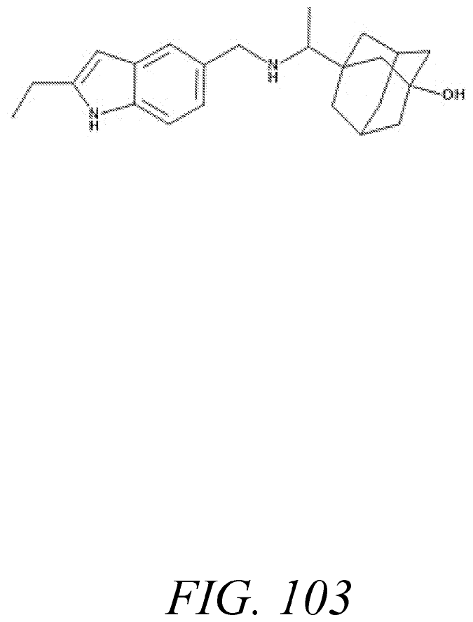

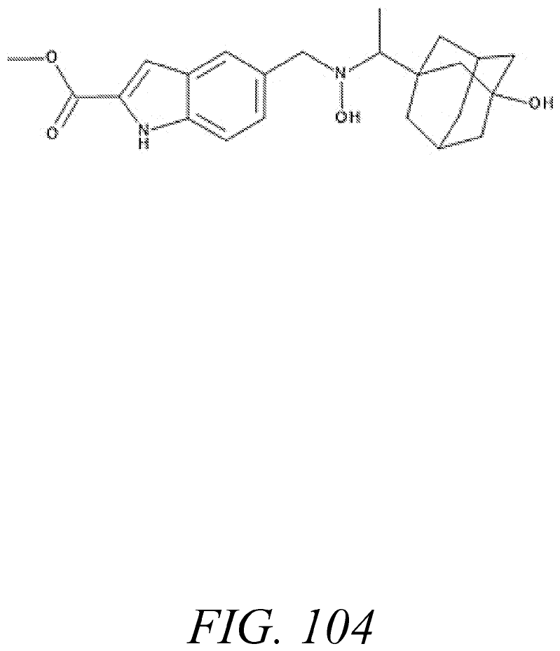

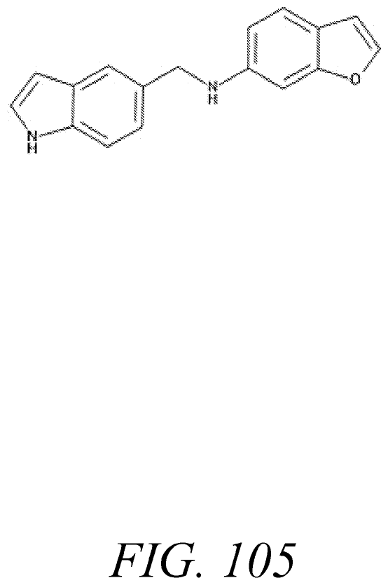

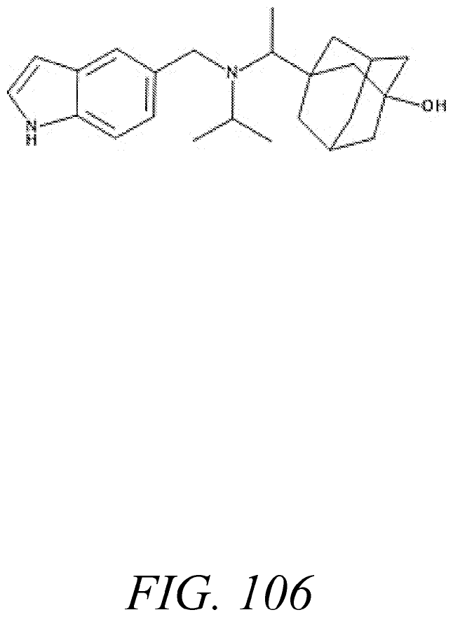

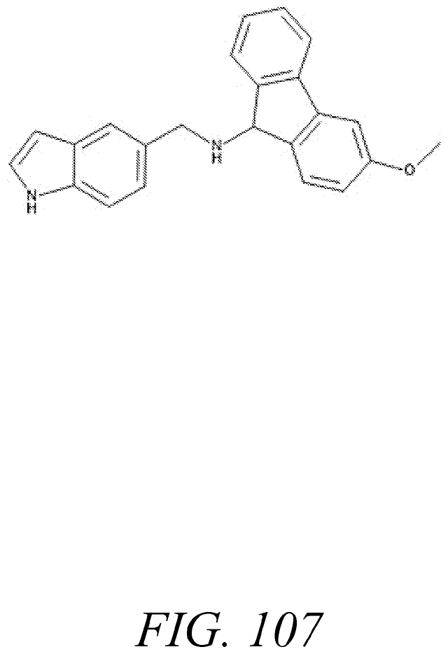

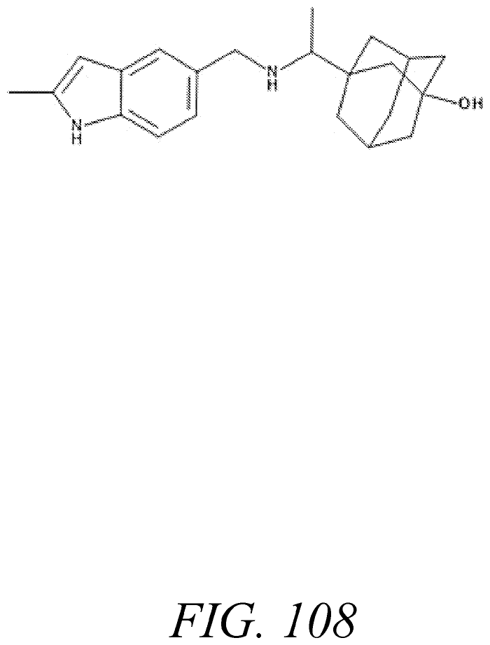

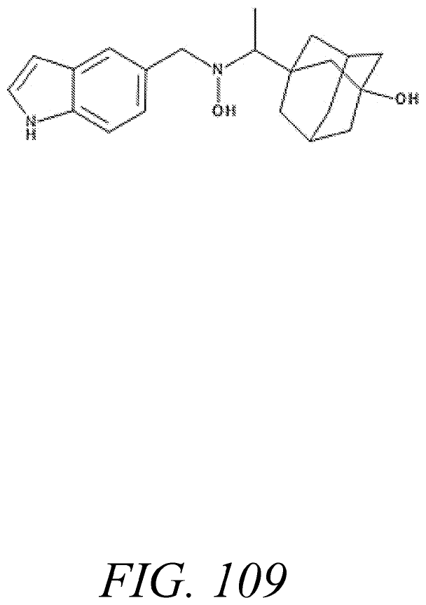

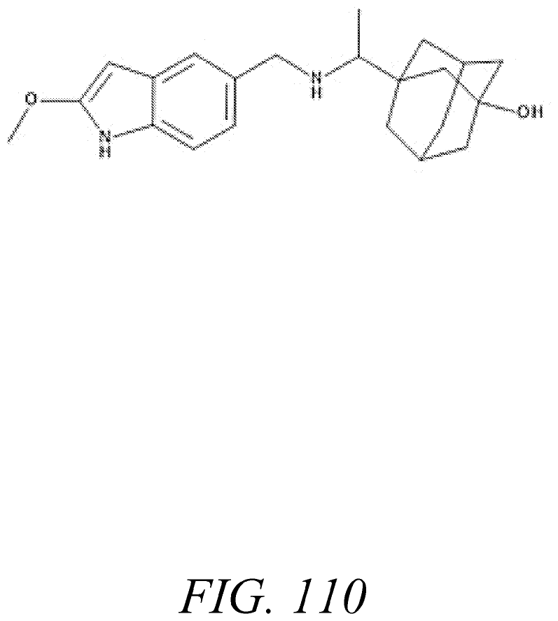

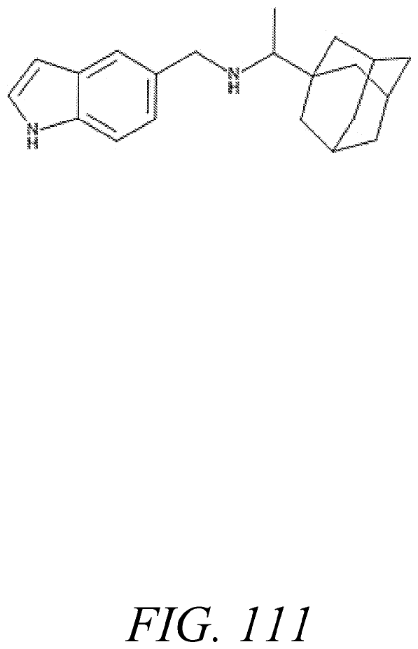

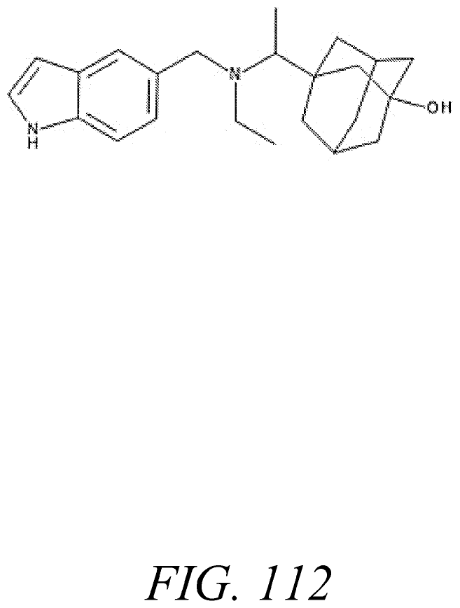

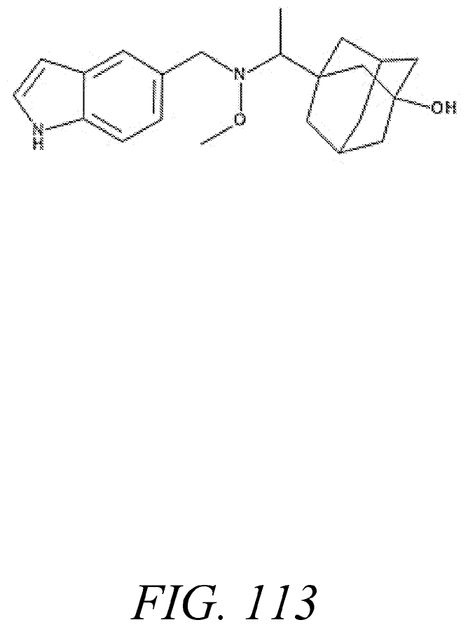

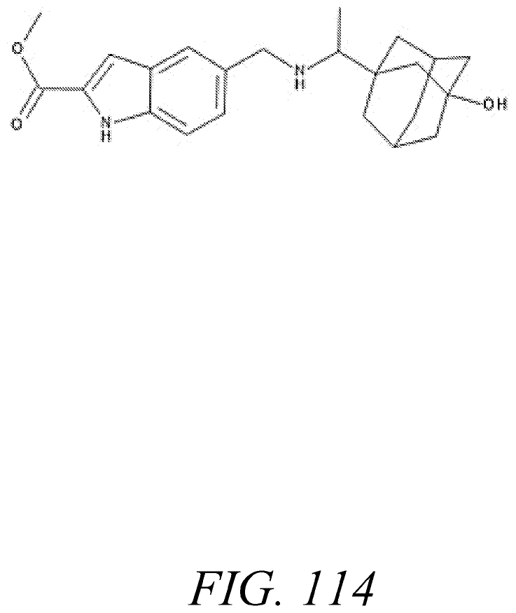

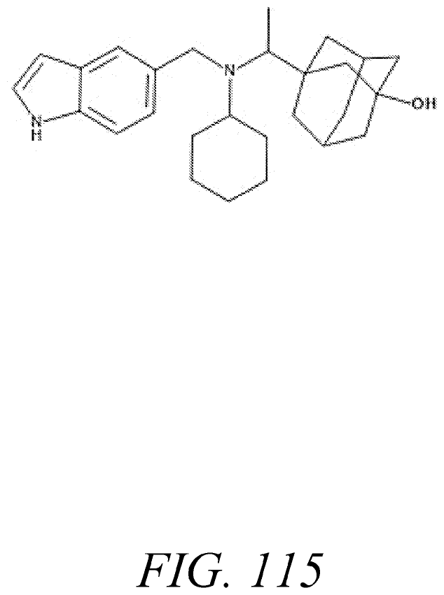

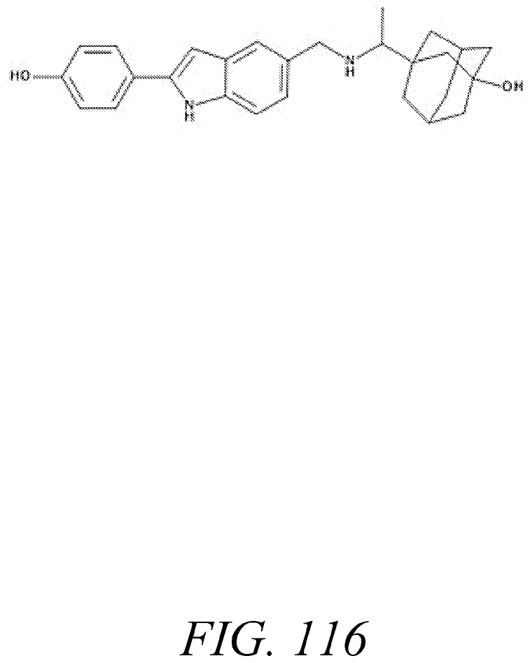

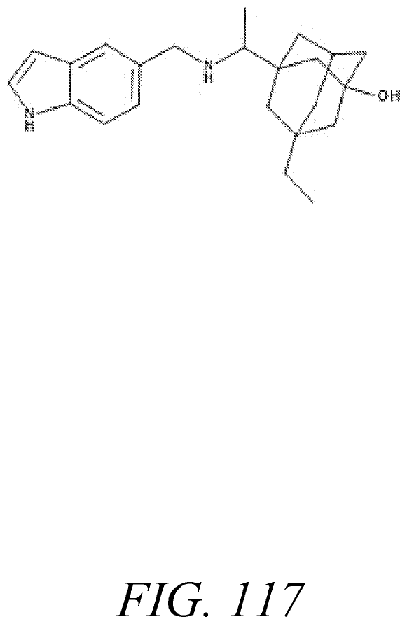

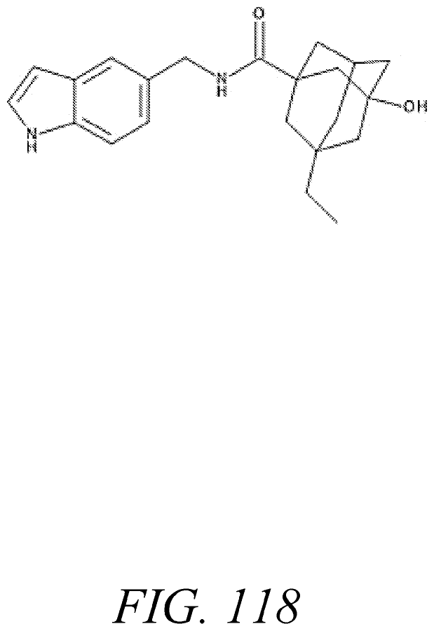

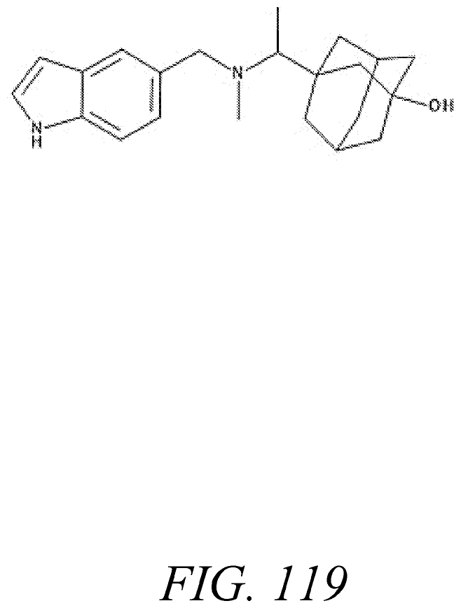

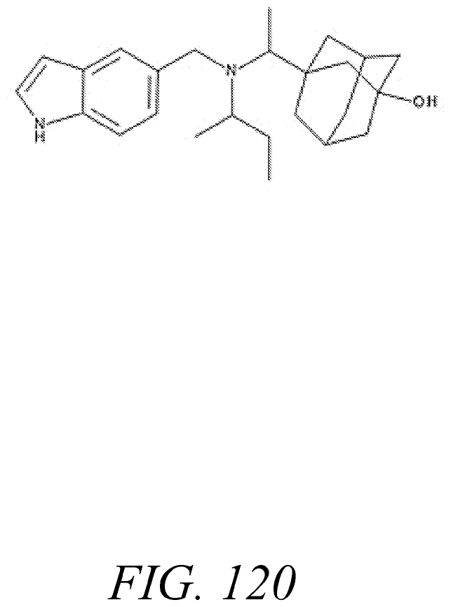

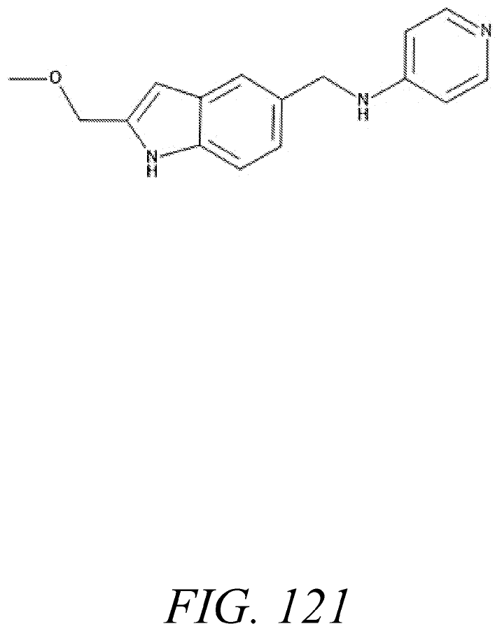

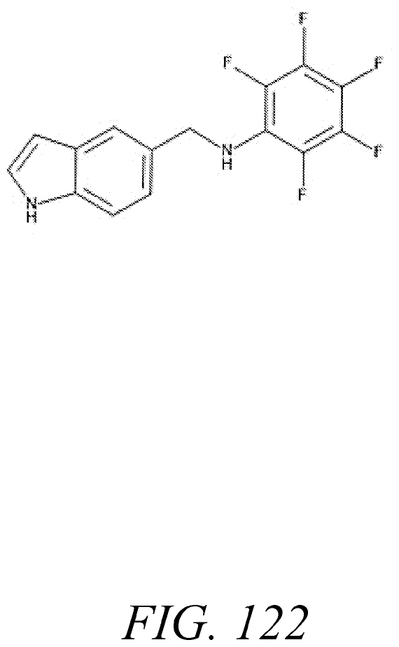

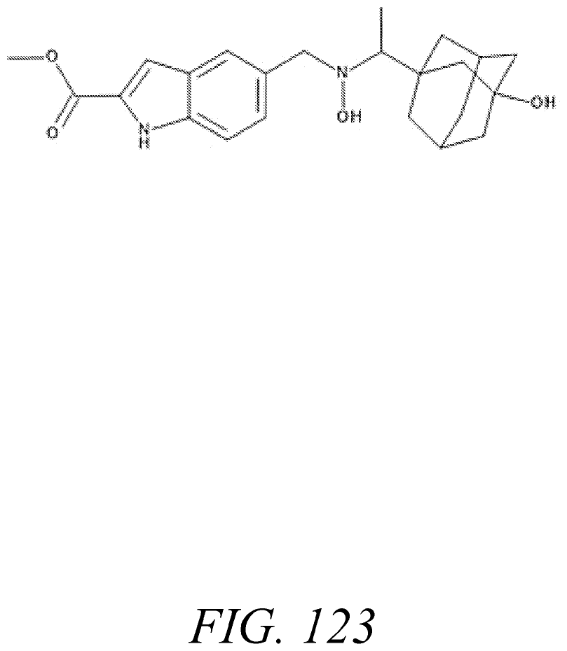

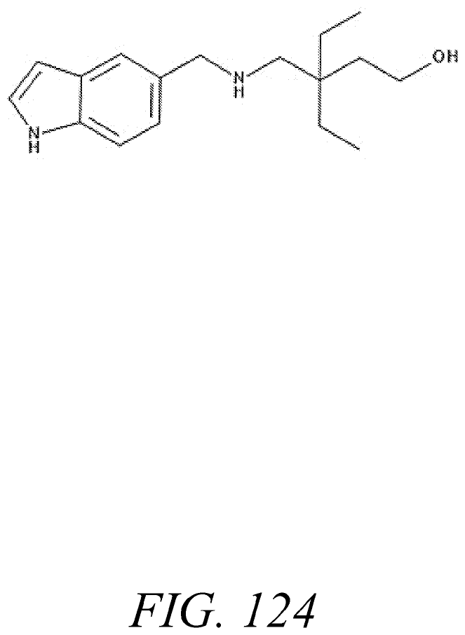

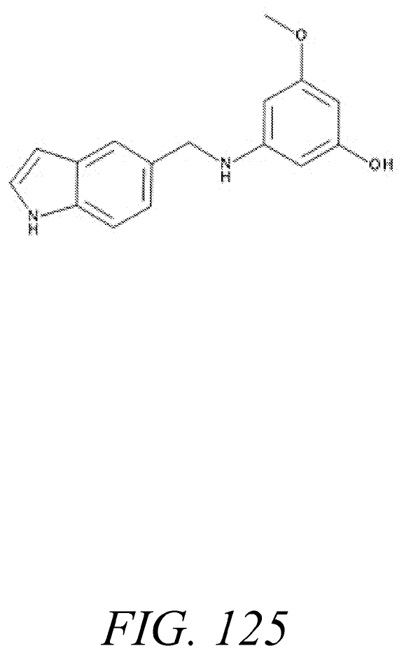

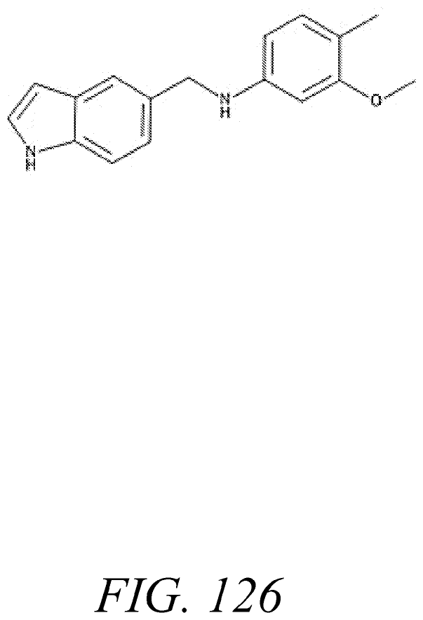

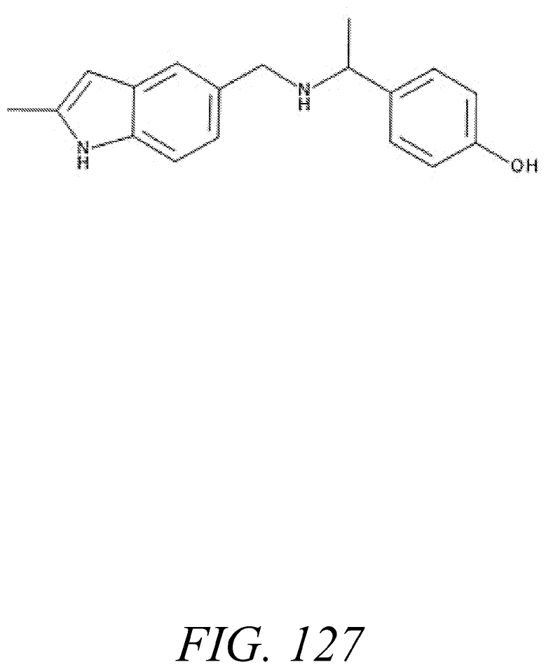

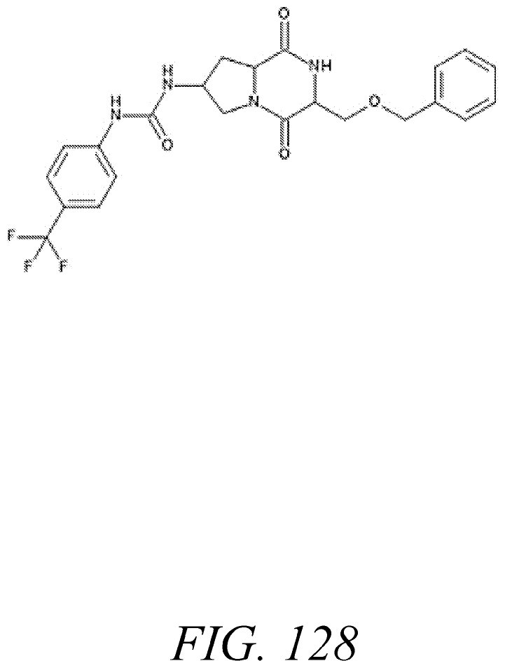

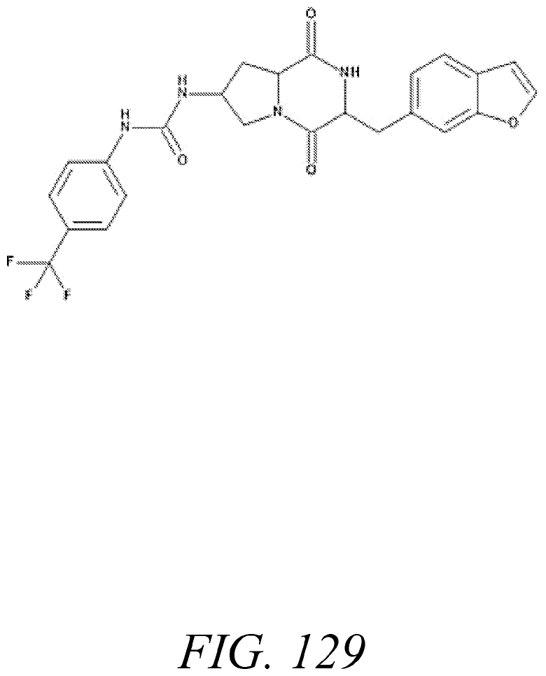

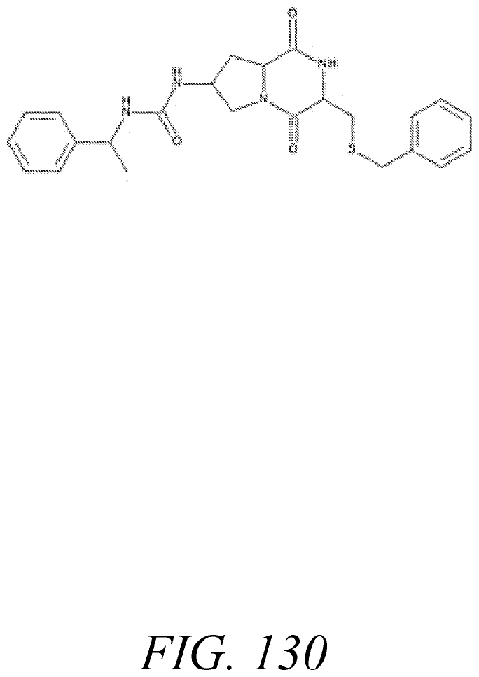

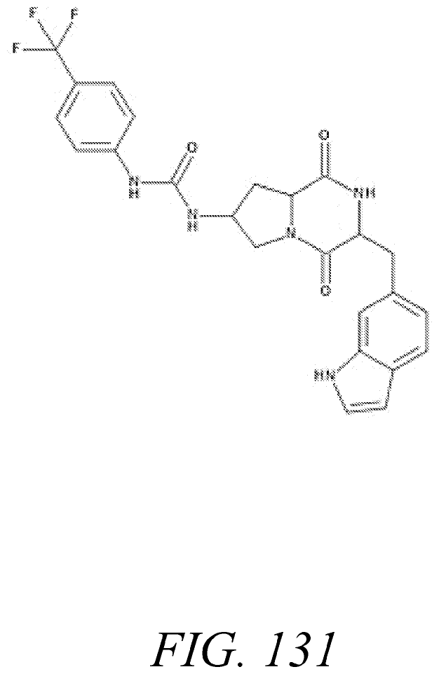

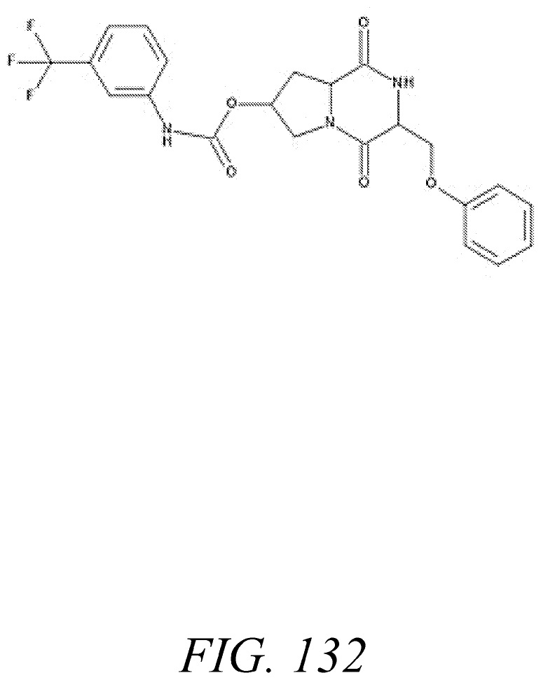

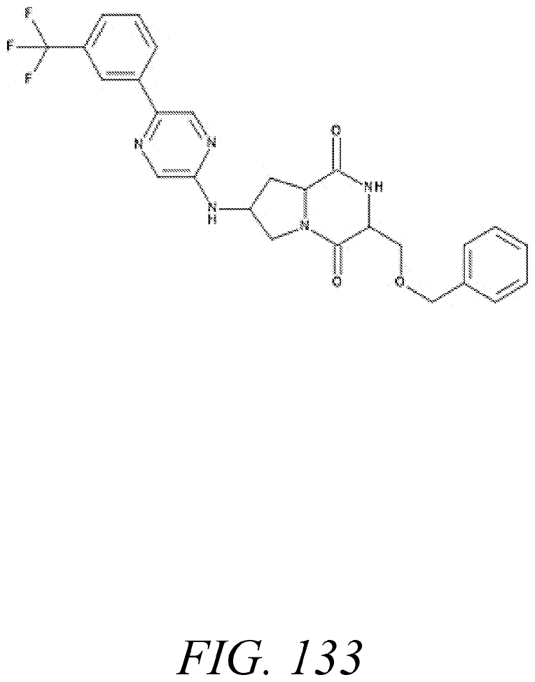

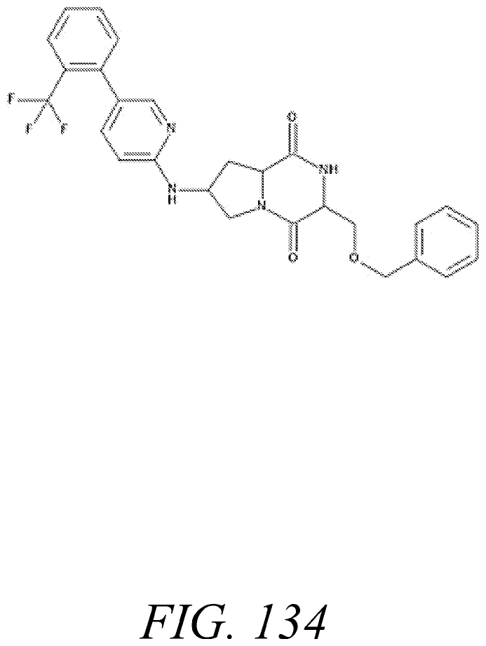

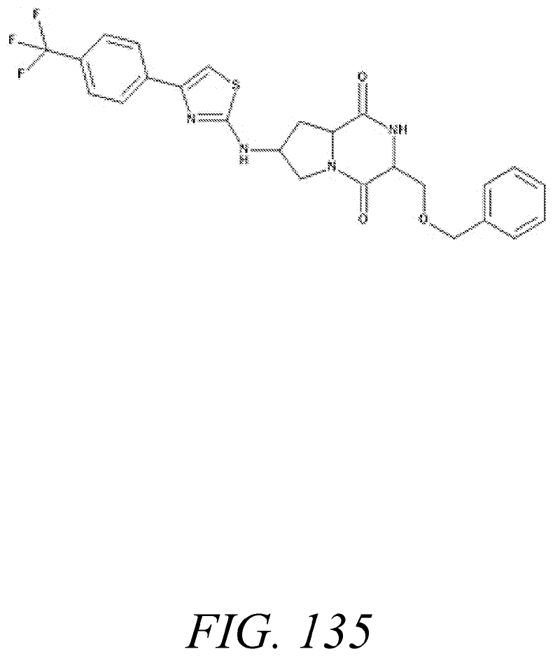

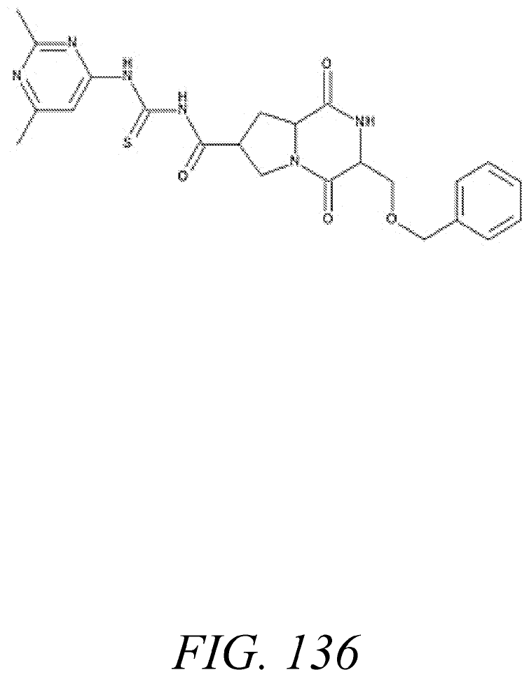

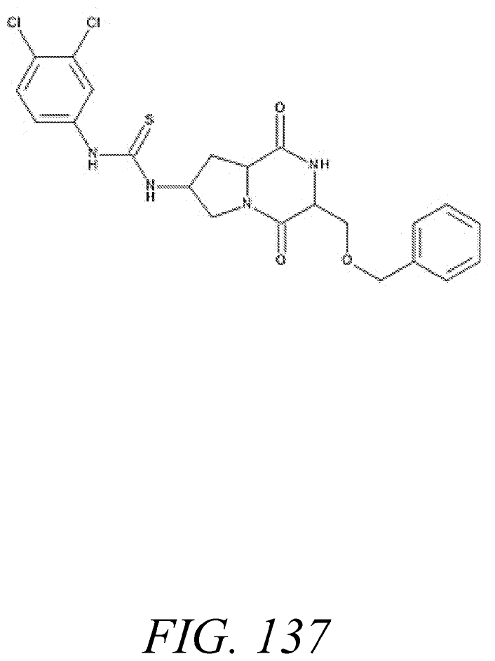

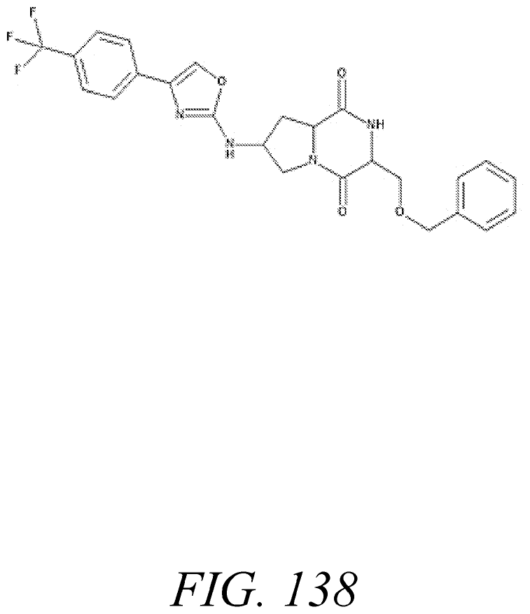

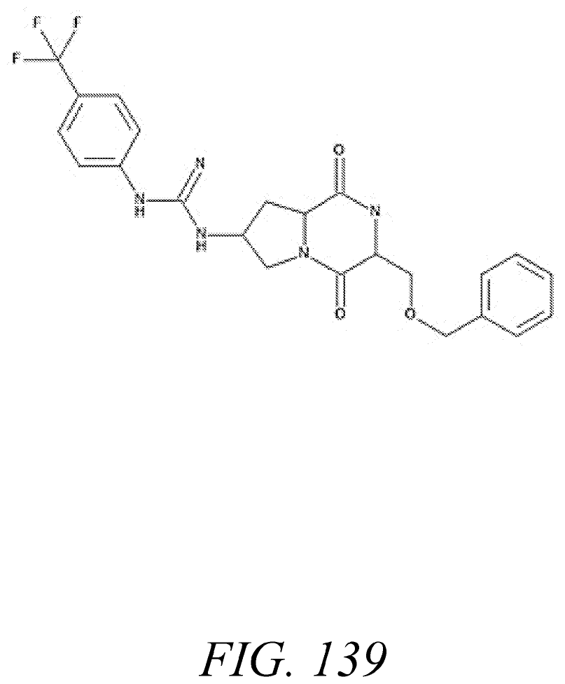

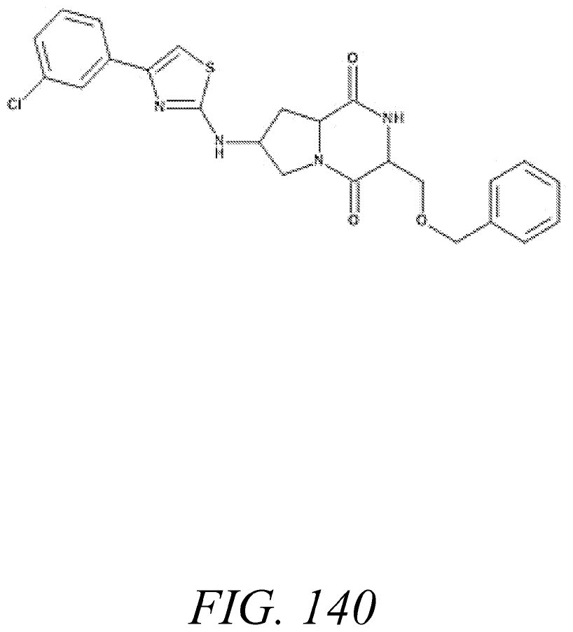

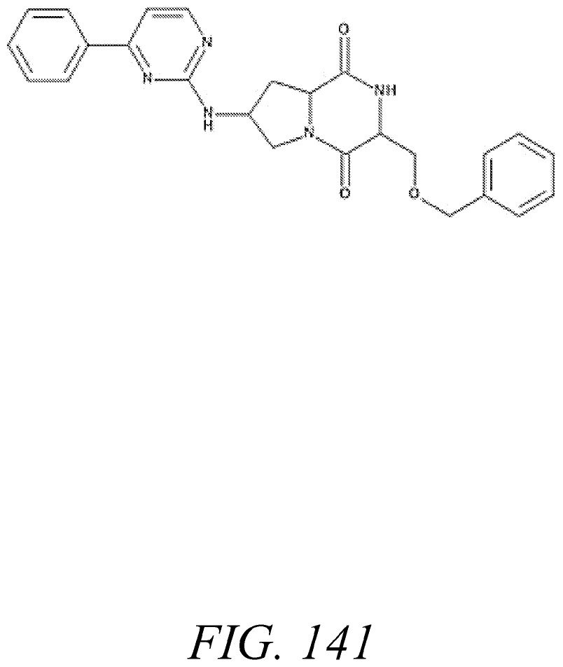

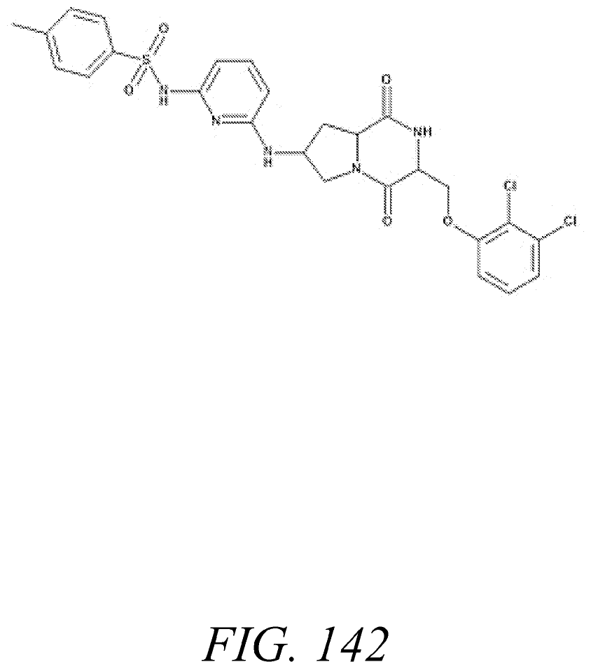

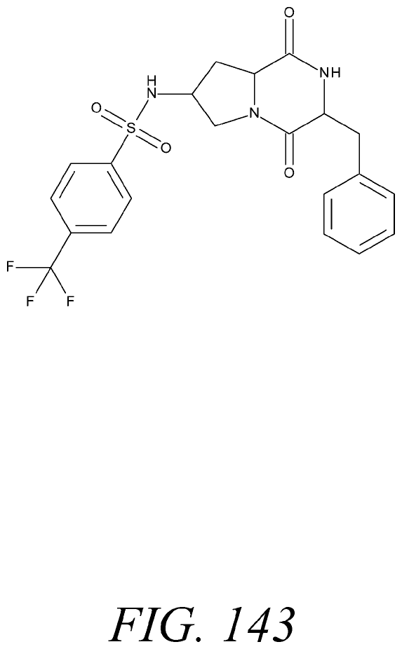

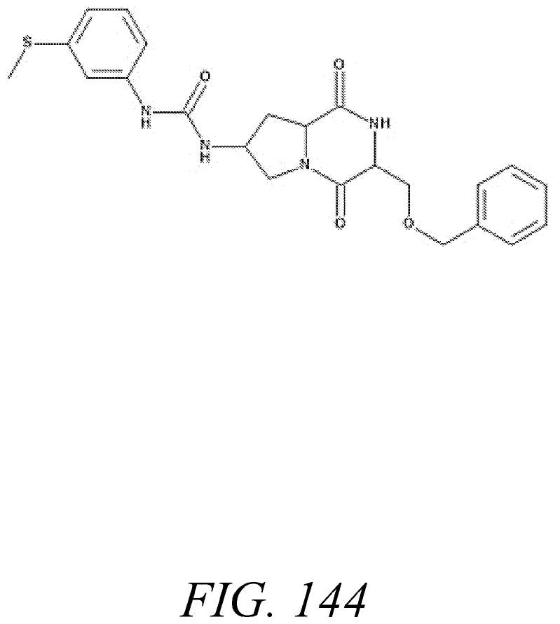

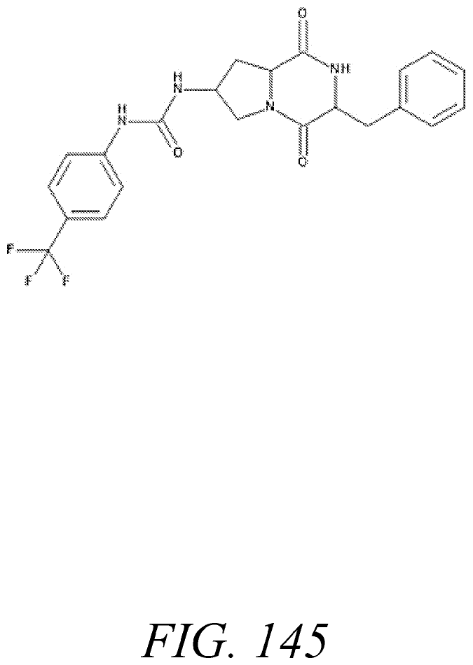

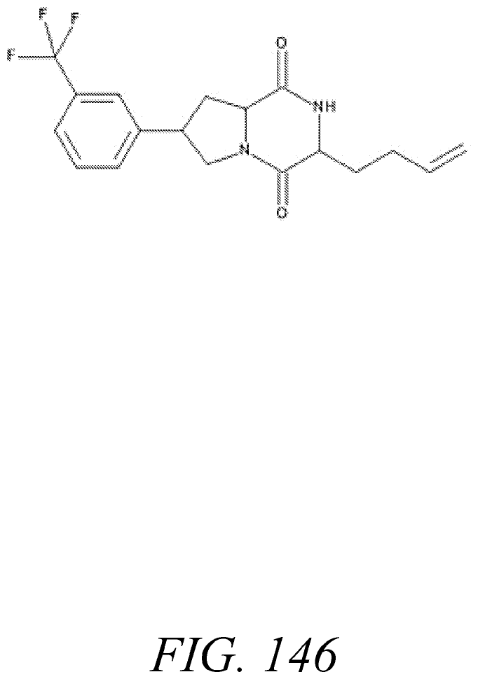

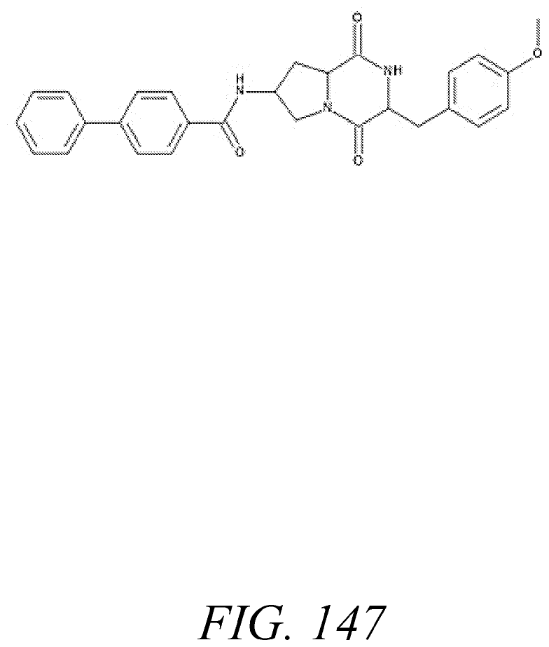

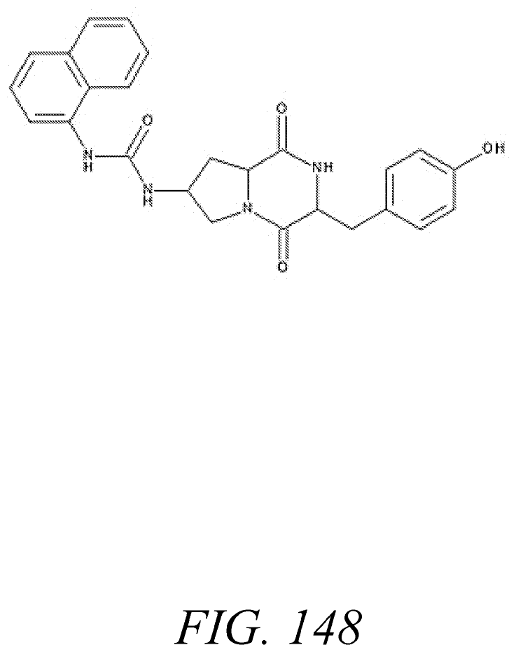

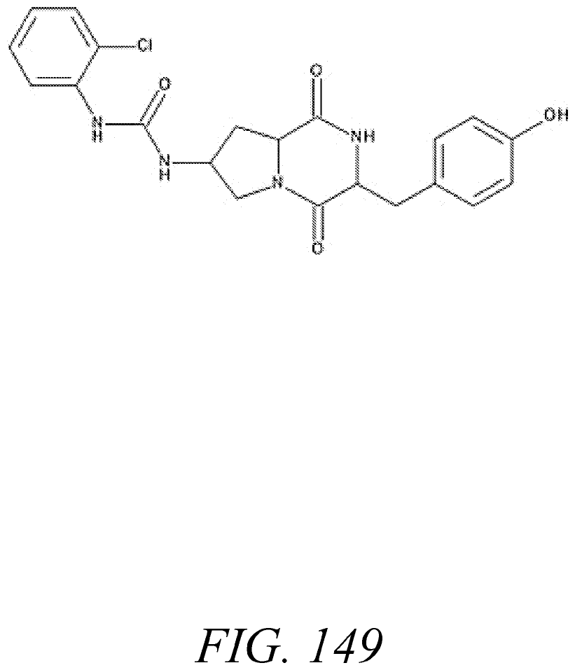

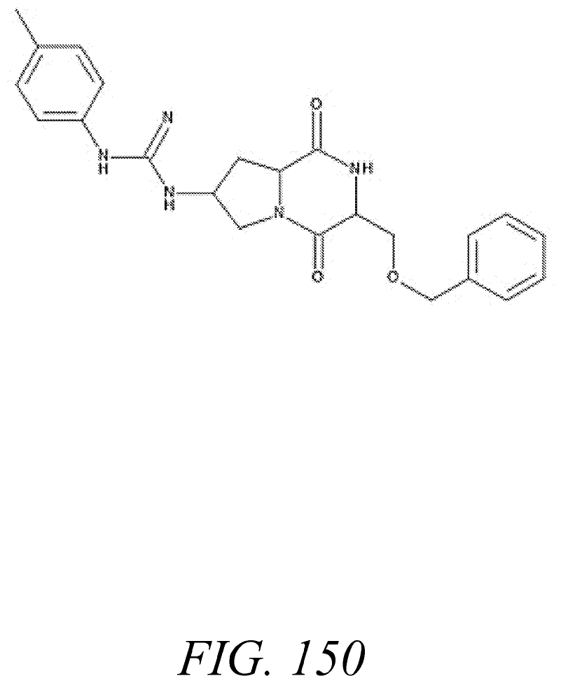

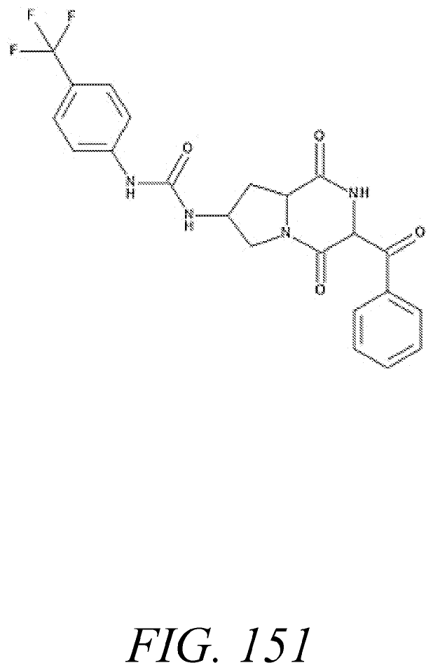

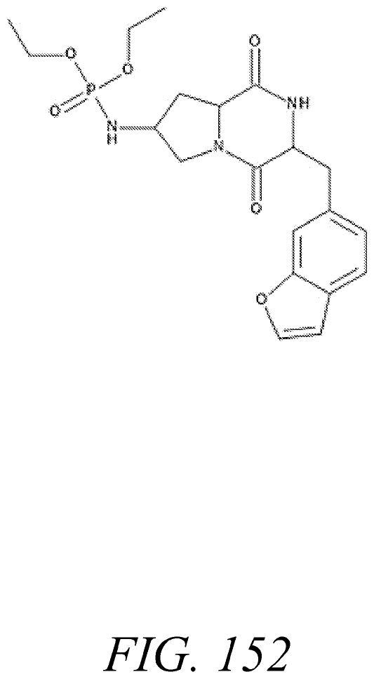

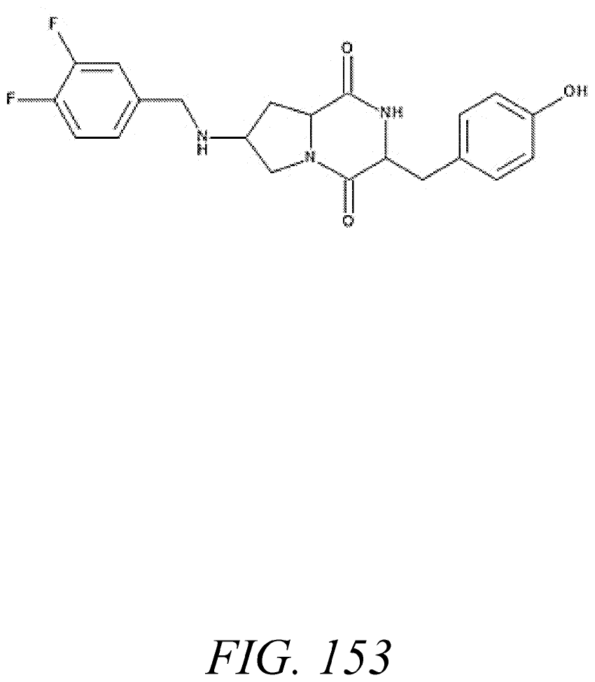

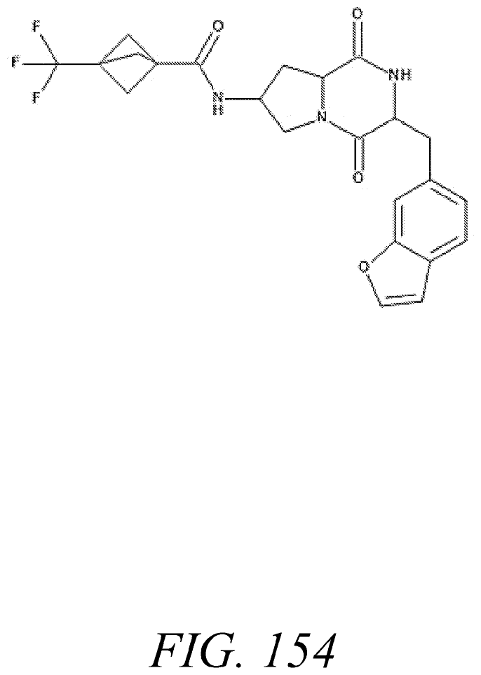

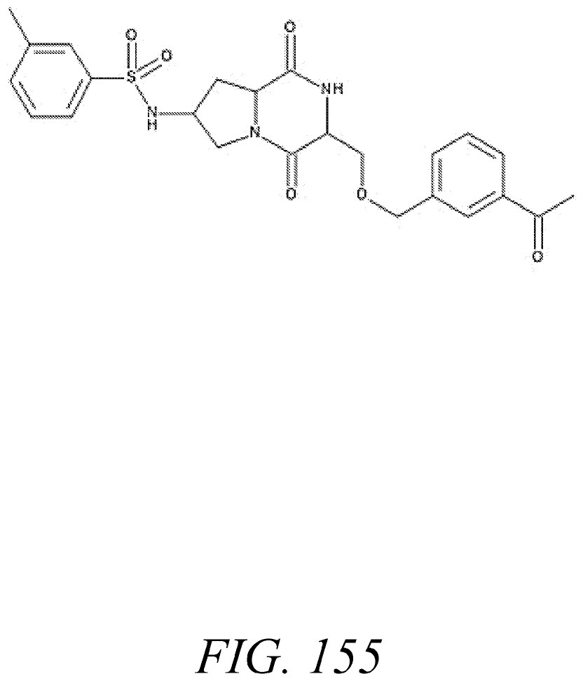

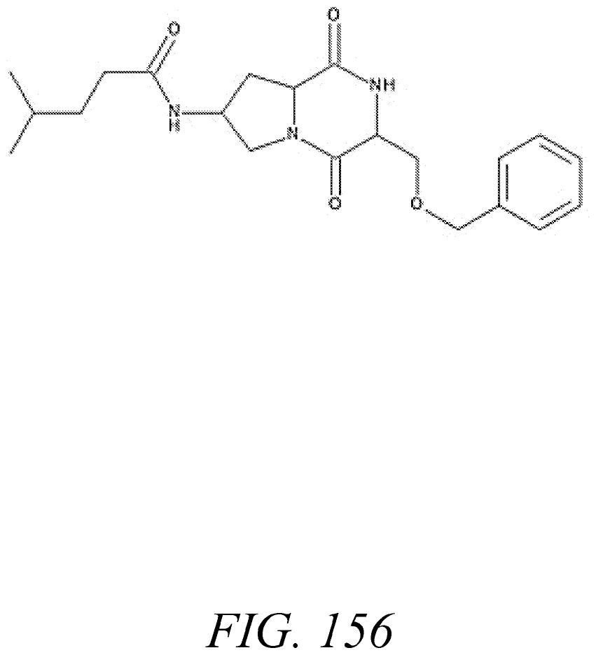

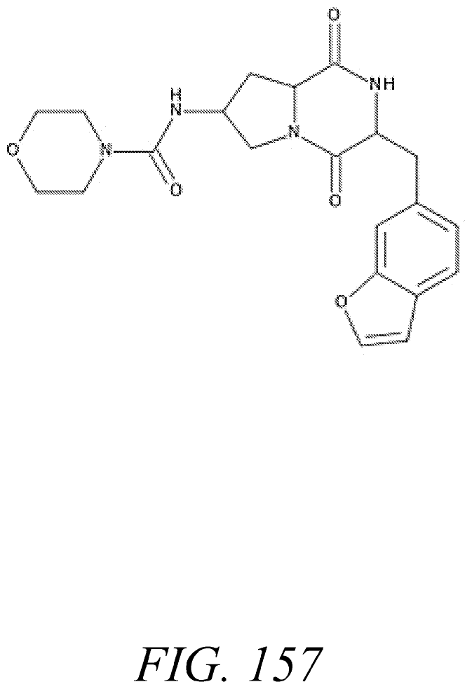

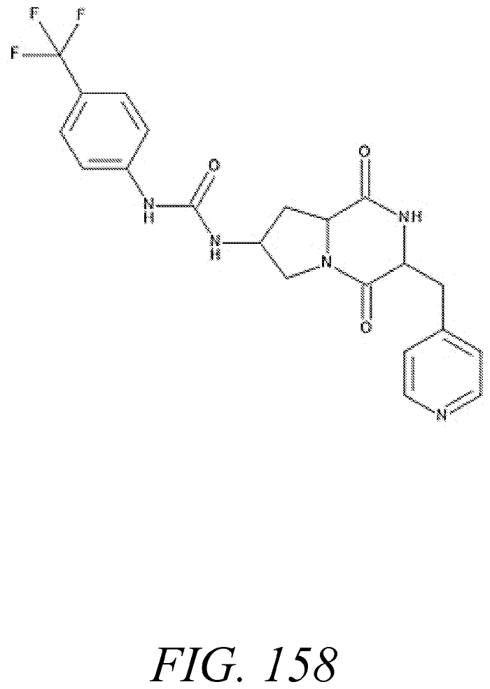

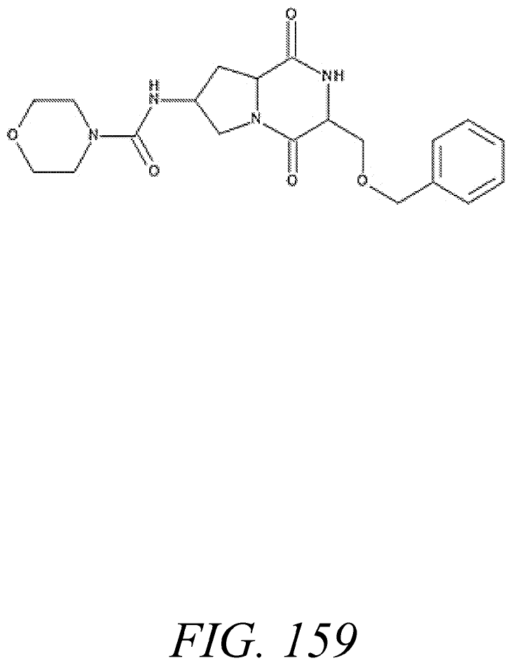

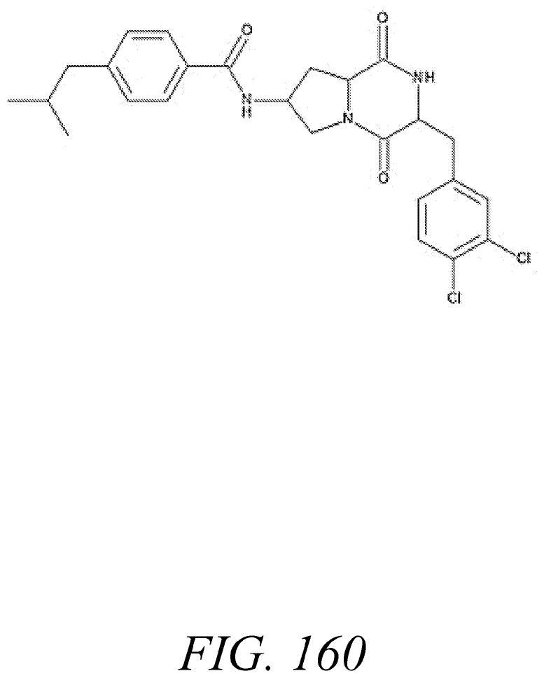

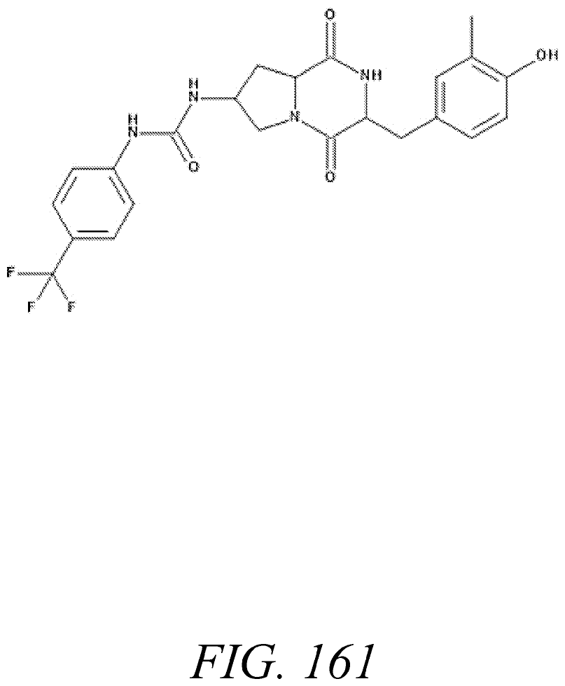

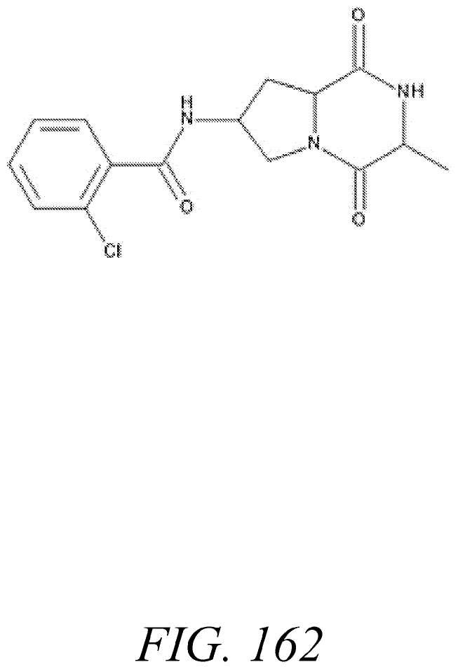

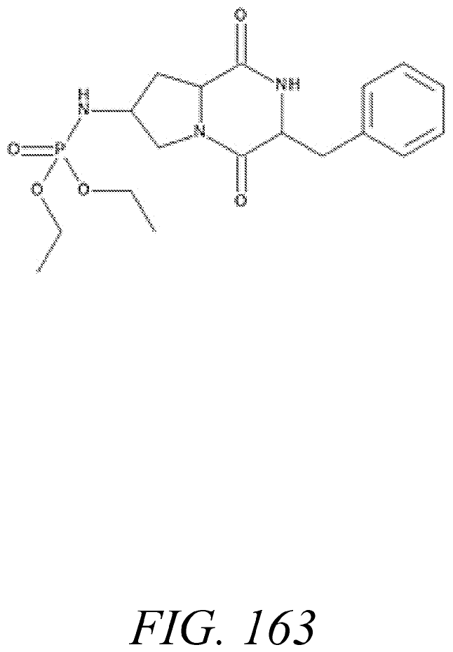

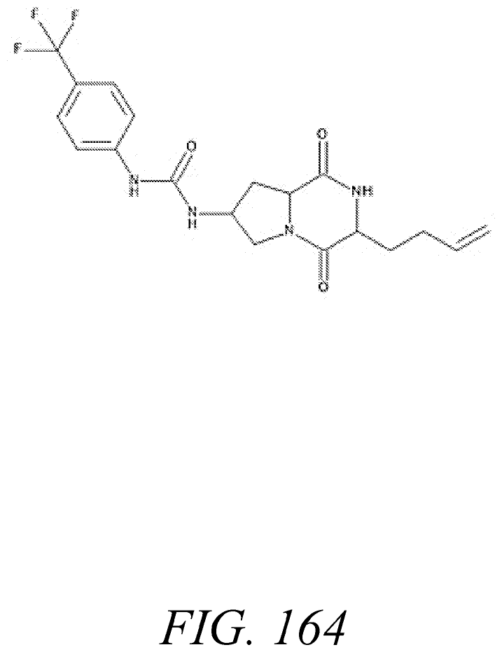

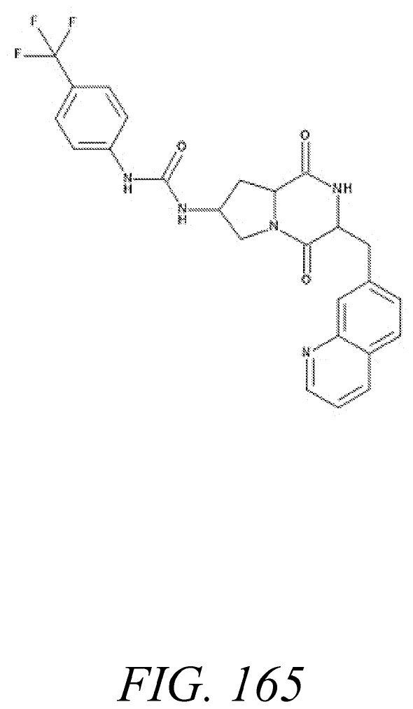

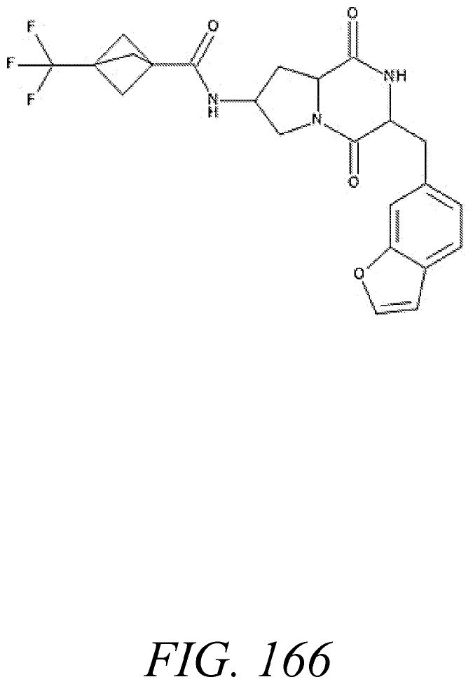

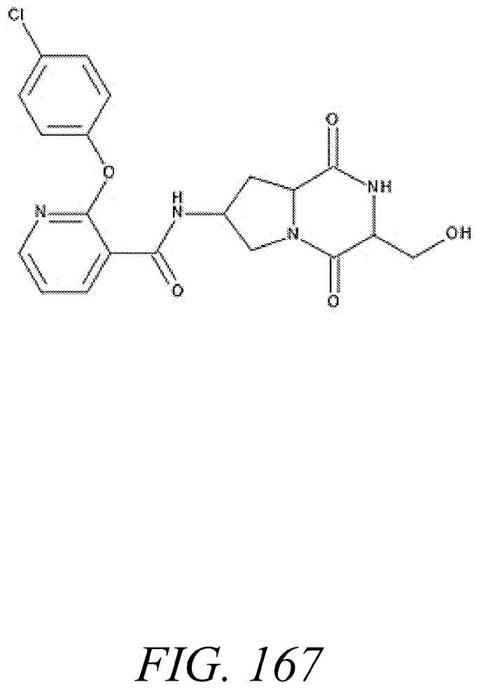

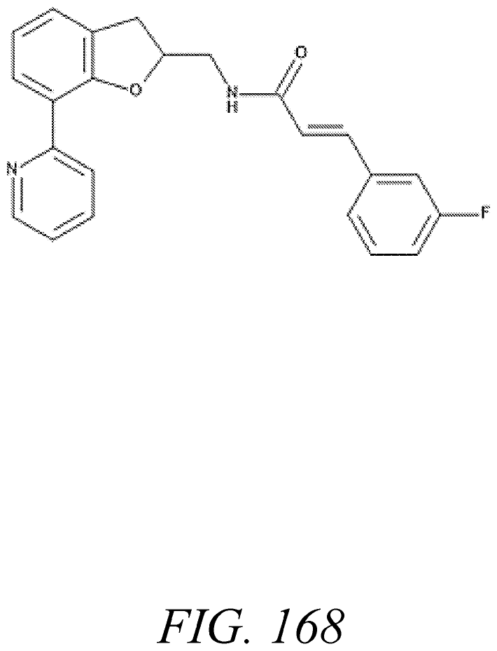

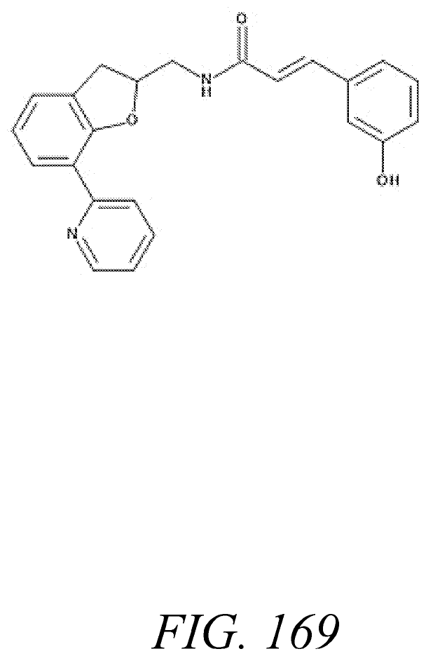

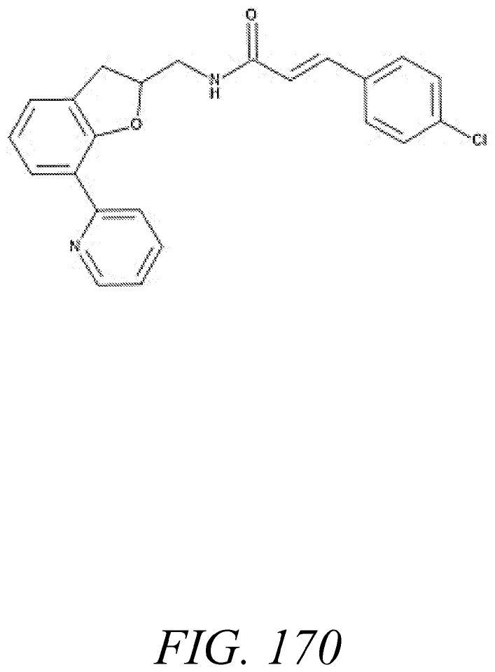

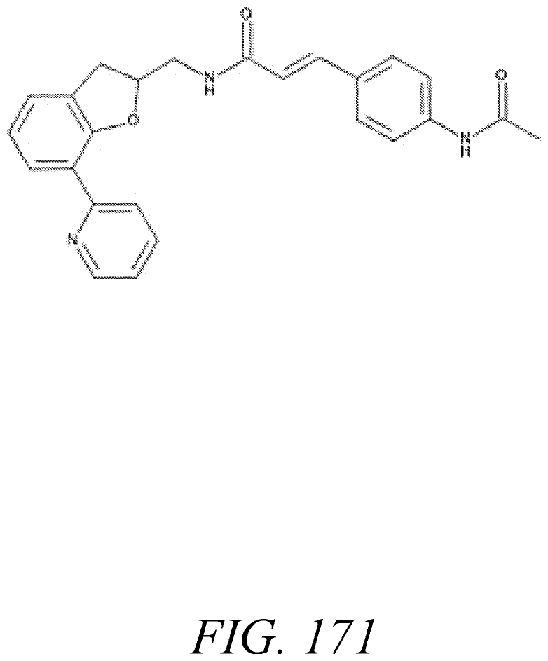

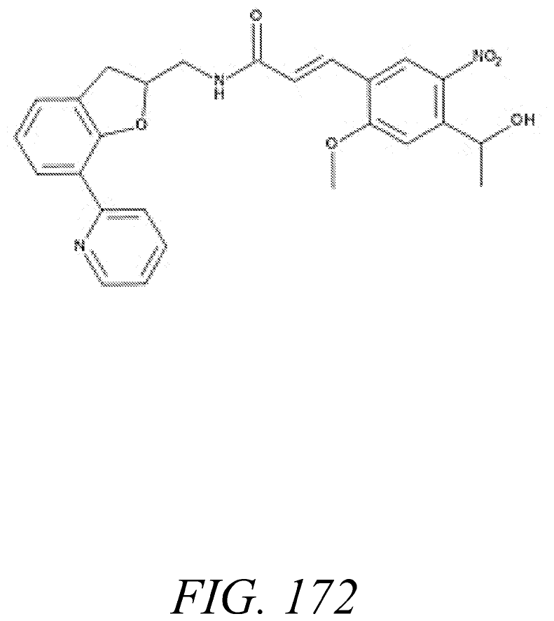

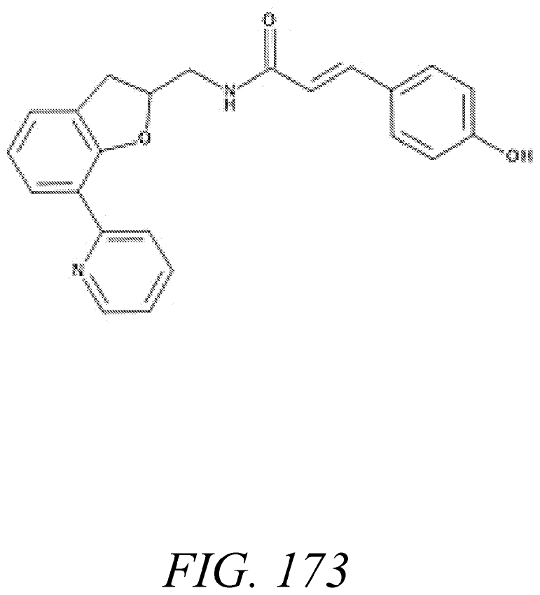

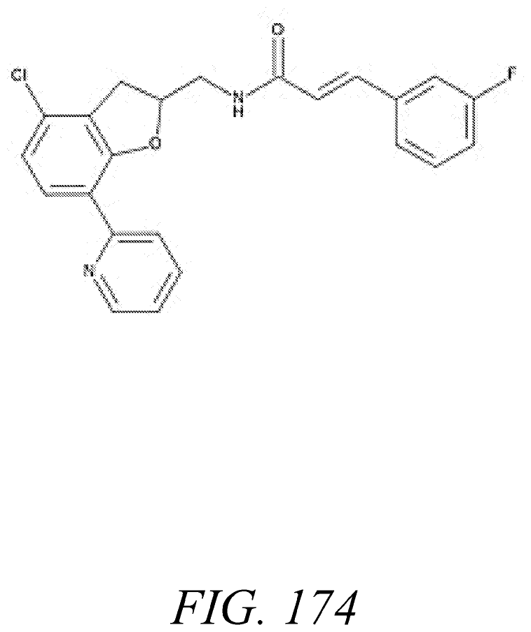

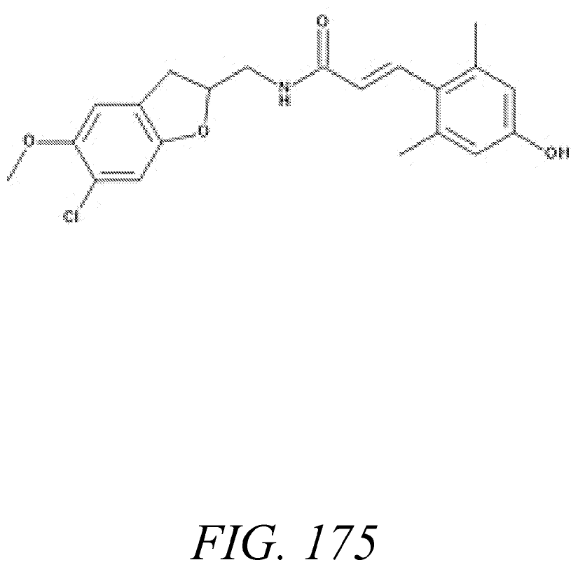

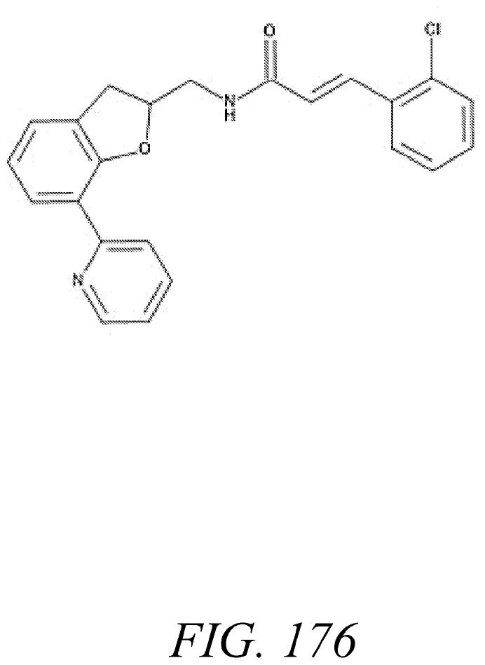

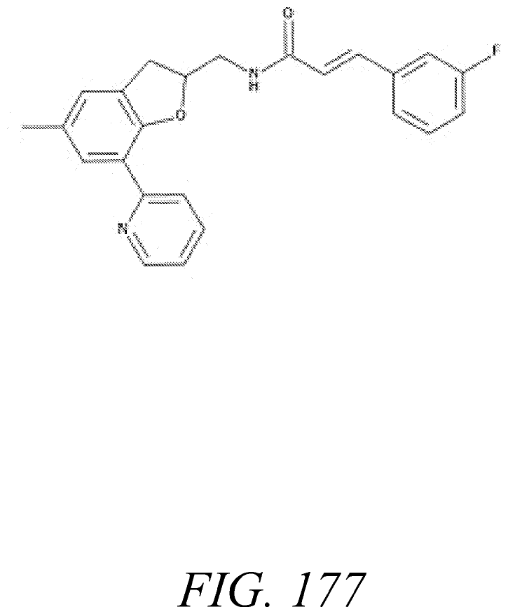

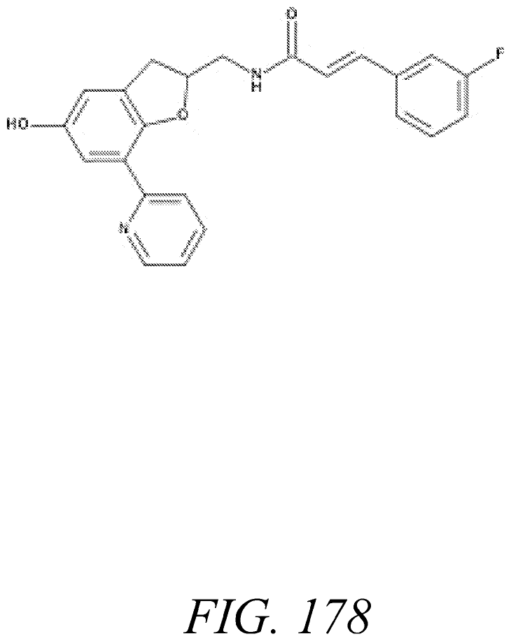

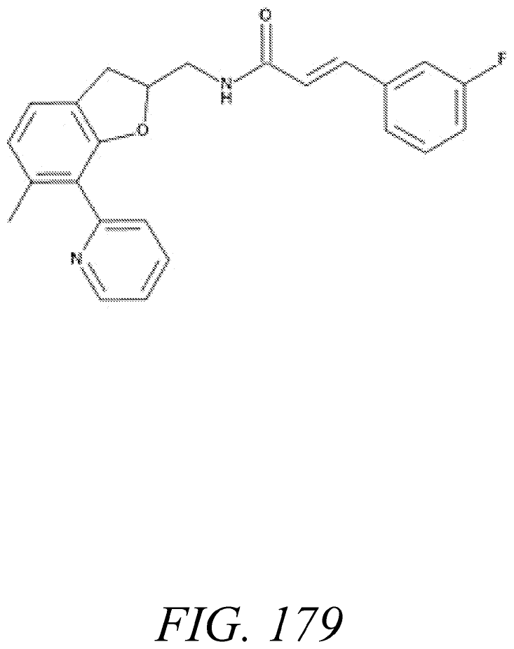

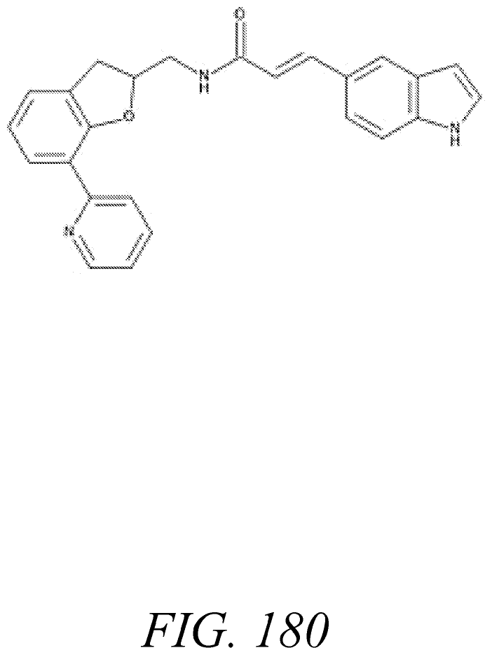

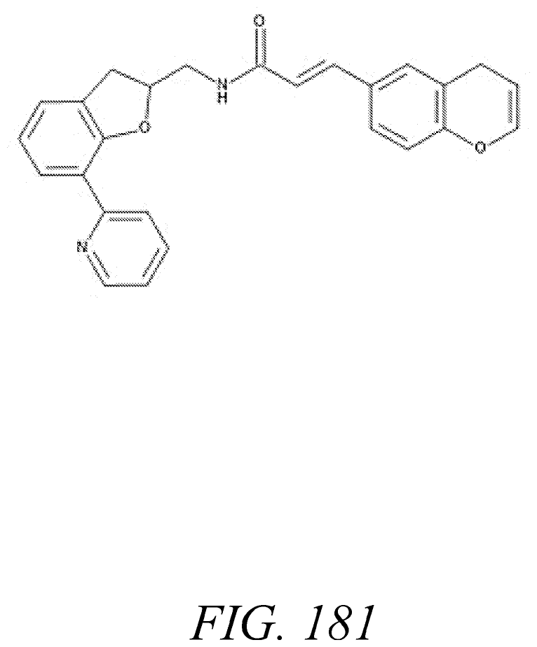

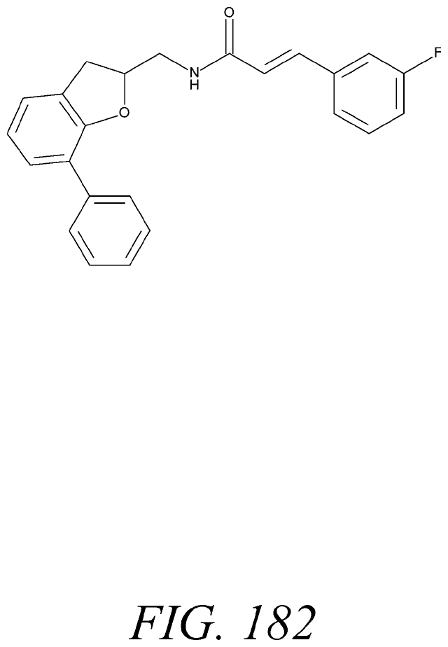

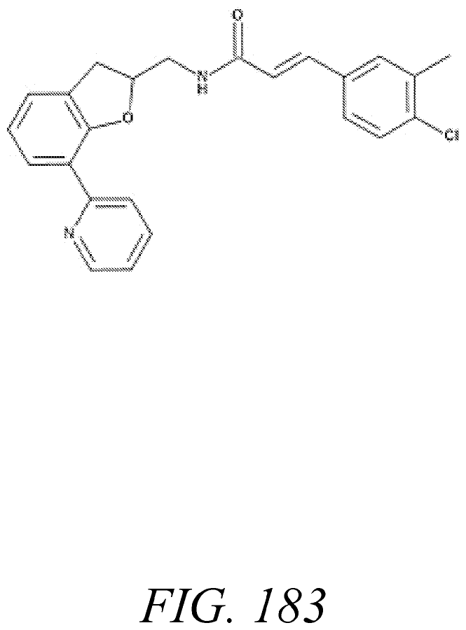

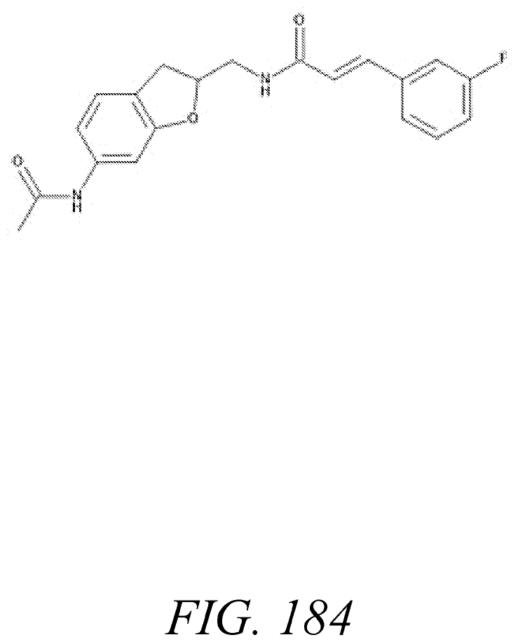

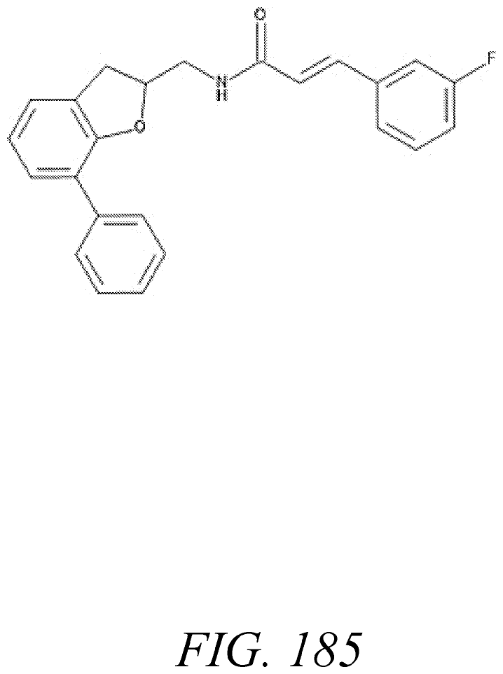

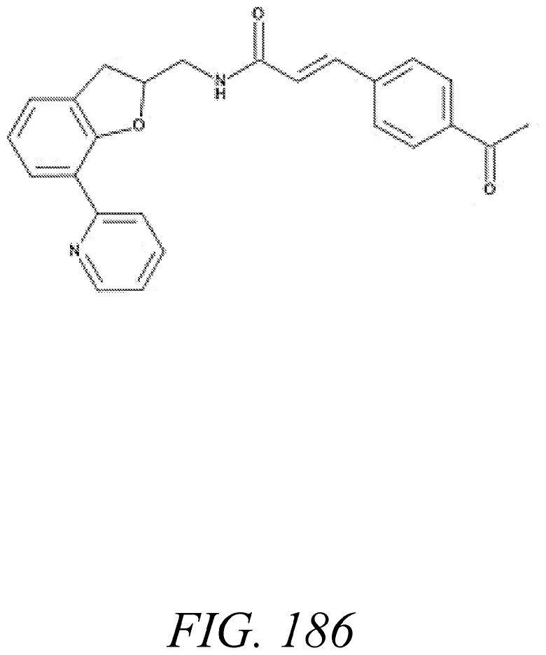

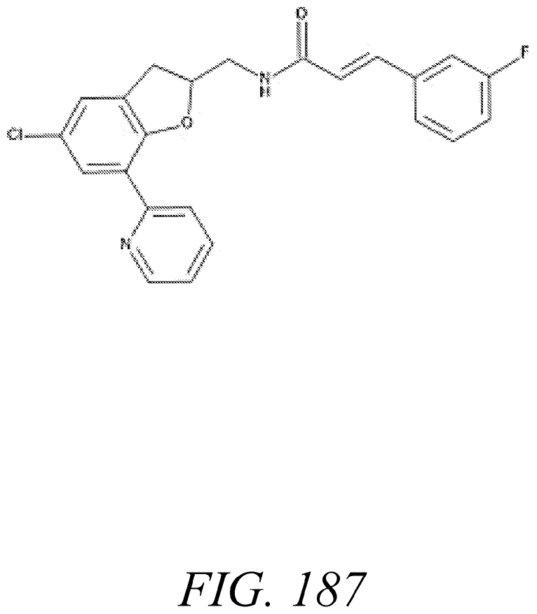

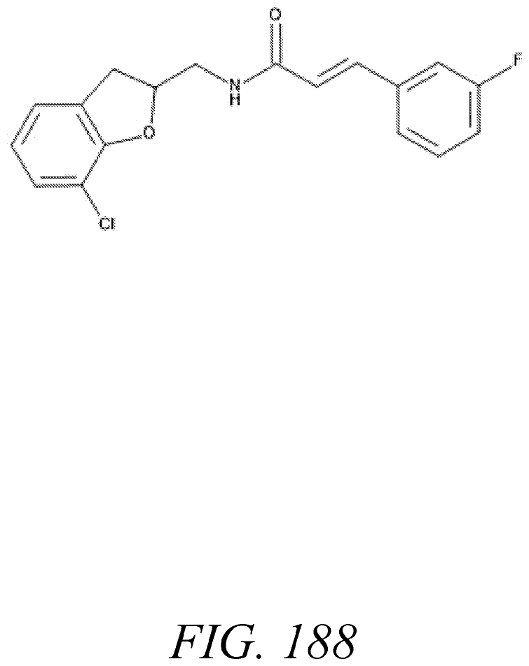

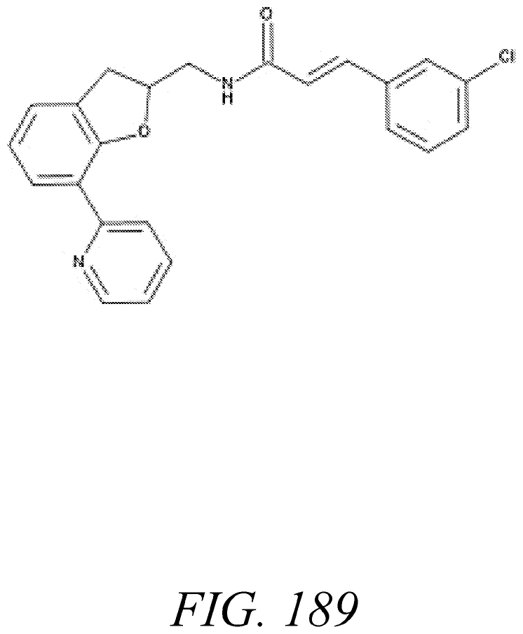

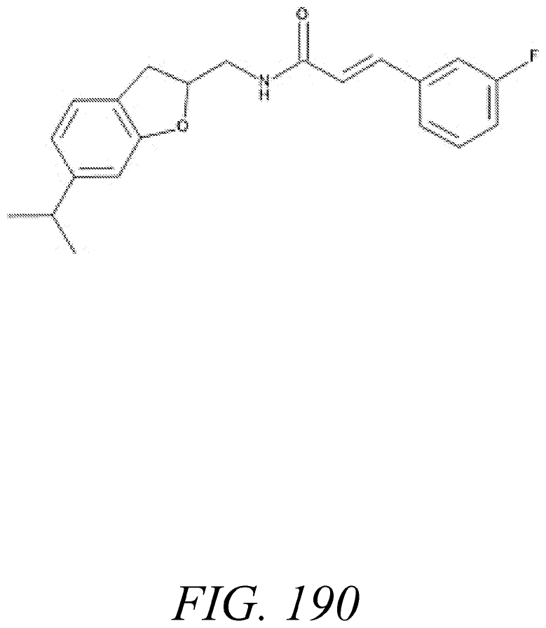

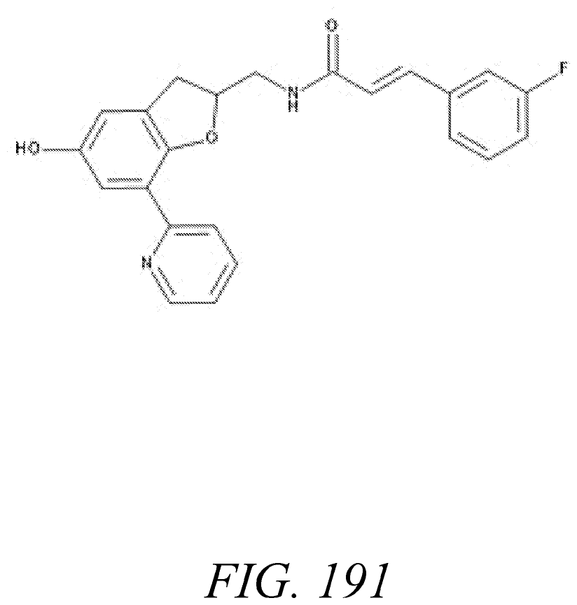

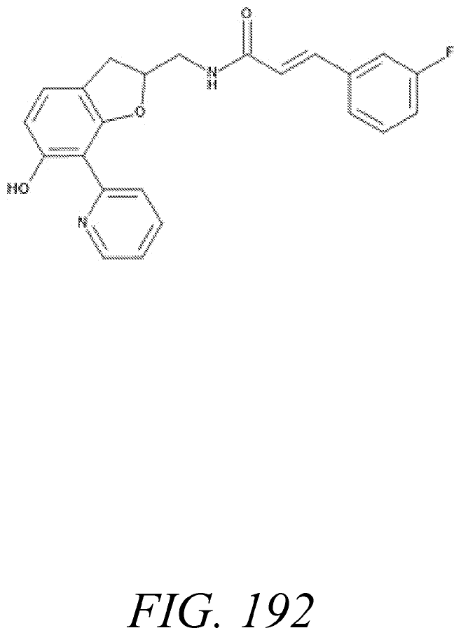

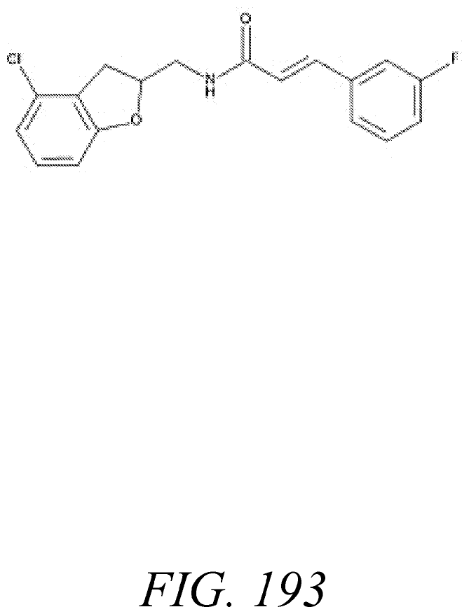

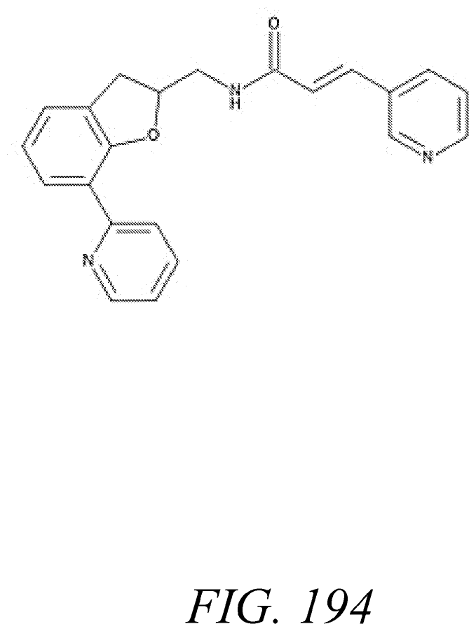

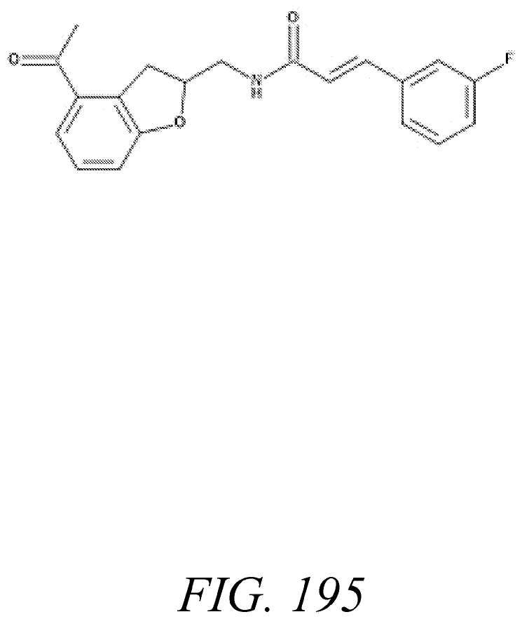

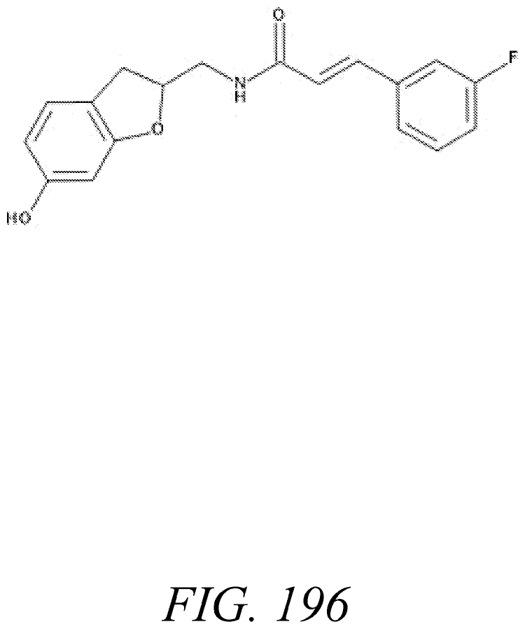

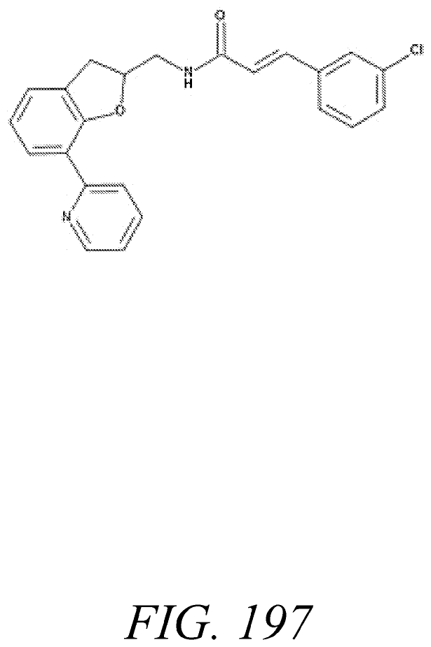

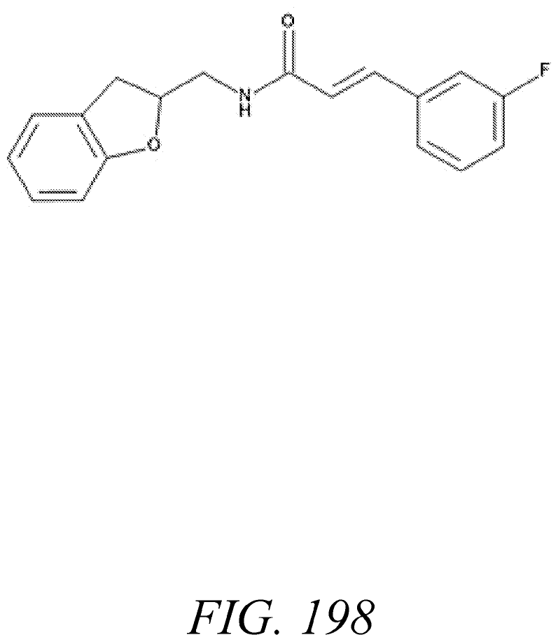

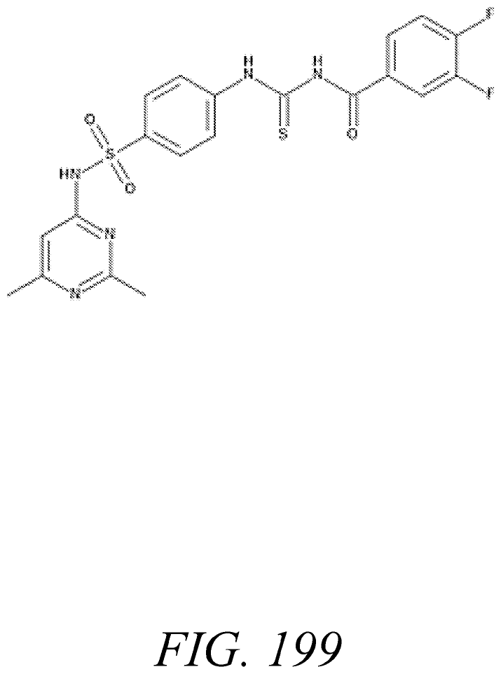

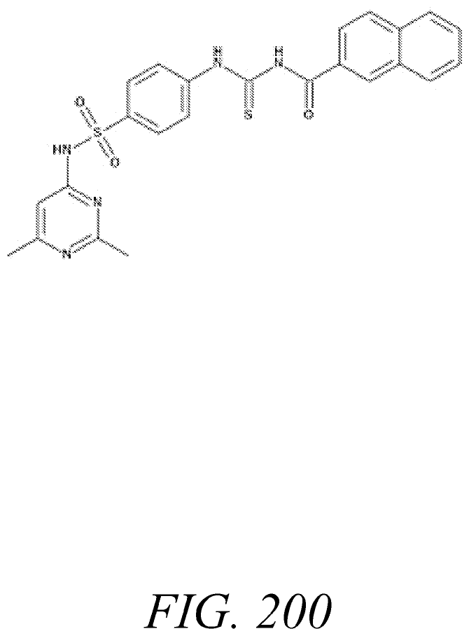

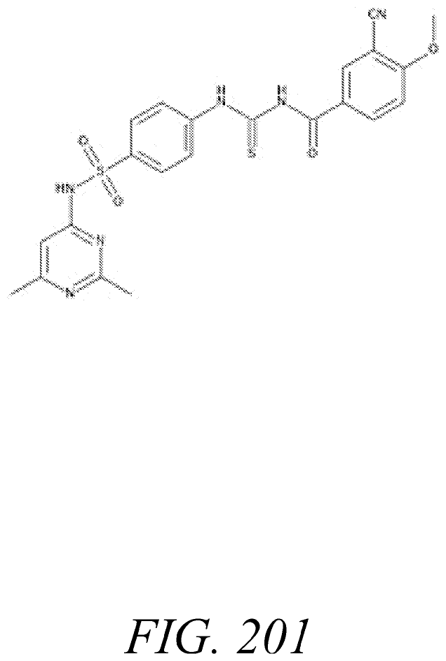

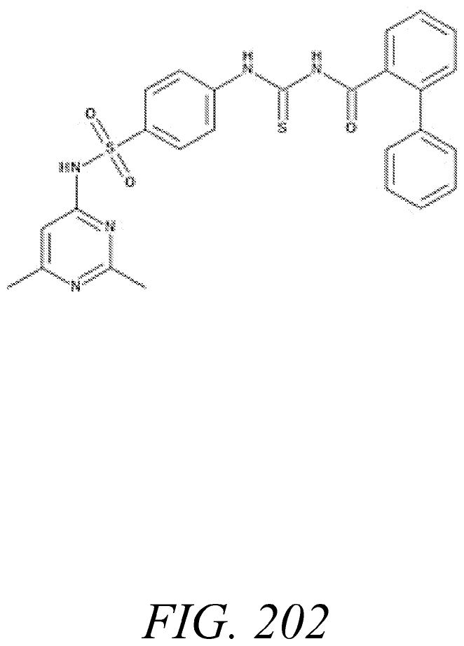

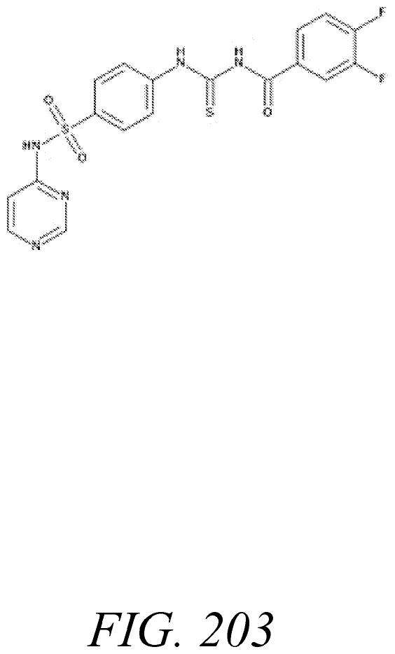

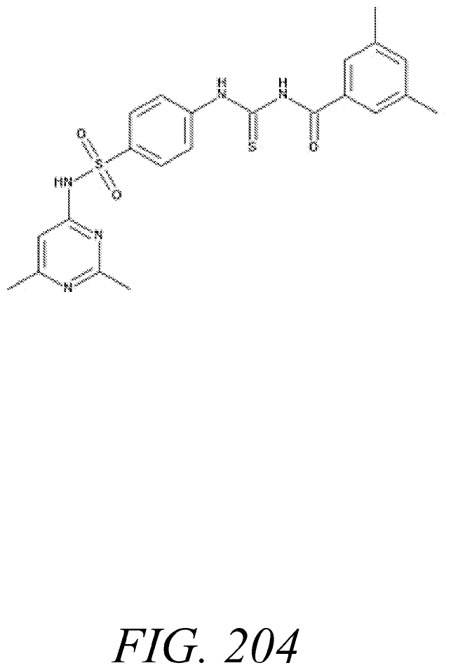

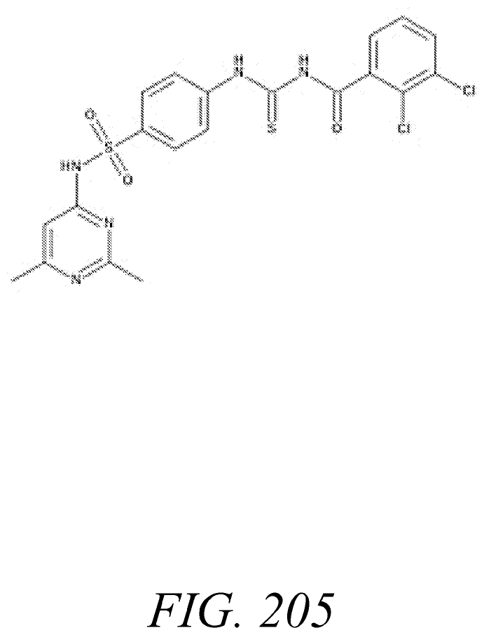

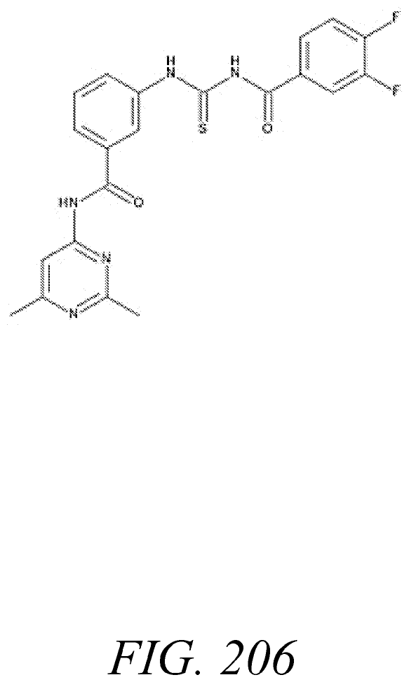

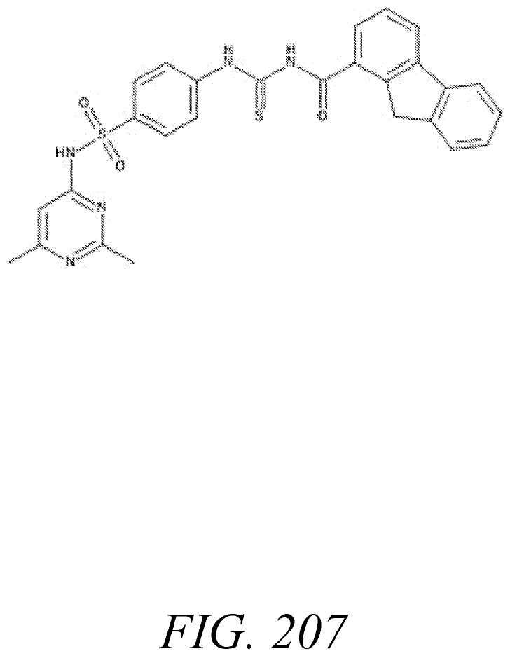

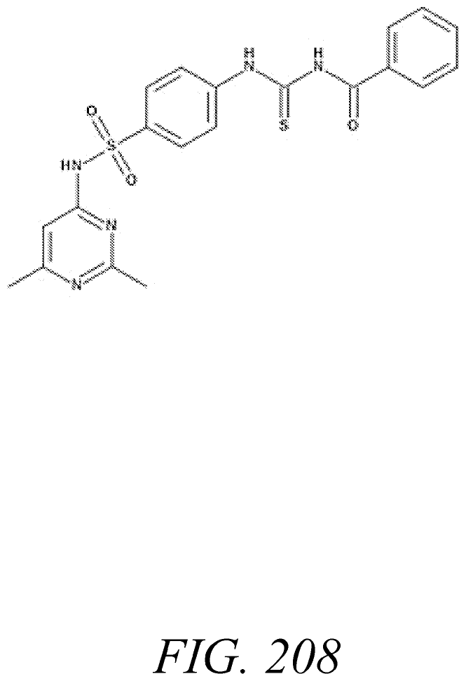

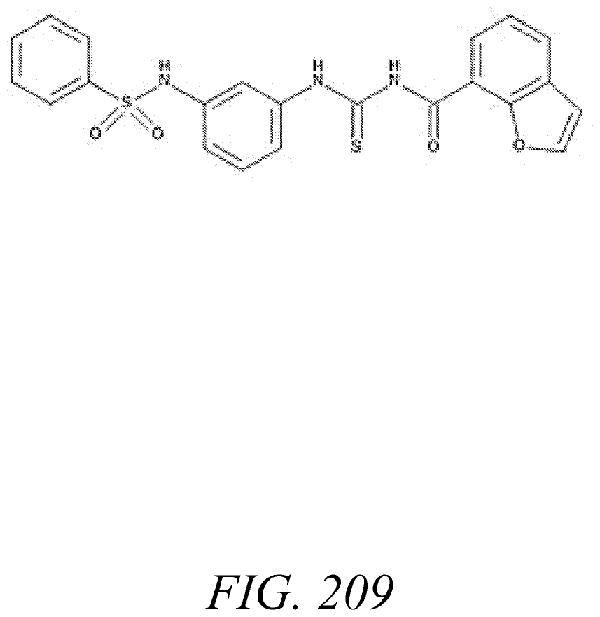

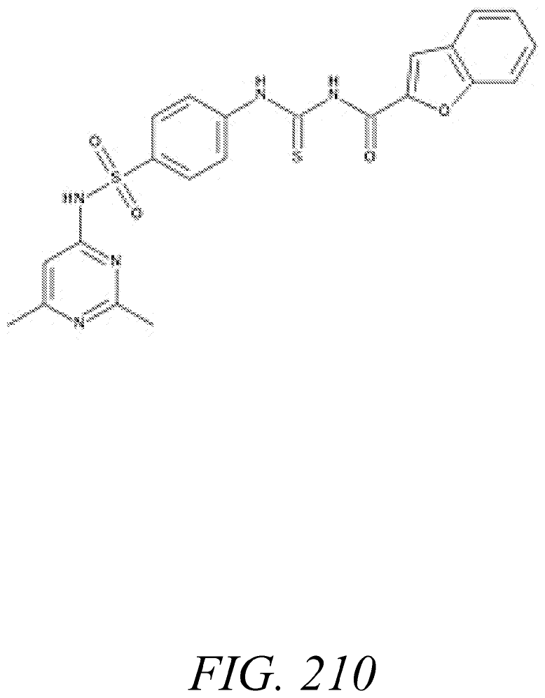

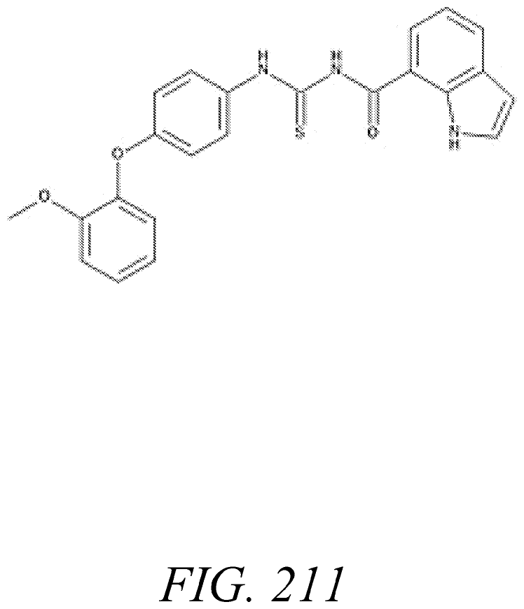

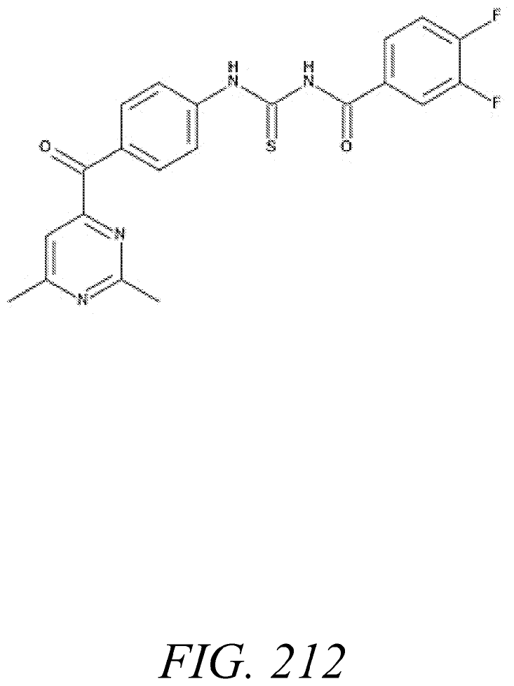

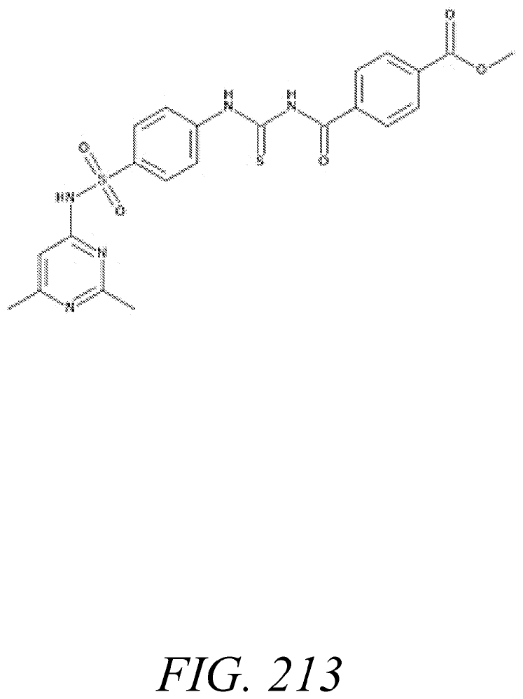

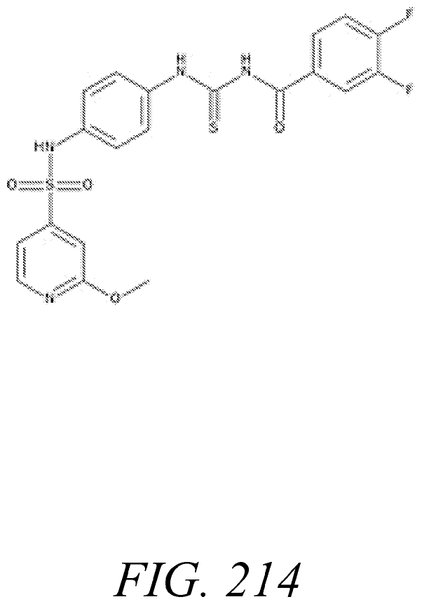

TABLE 3
Docking scores of, and in vitro SIRT3 modulation by, the top 20
compounds selected through virtual screening (hit compounds*).

| | | | | | % Control in vitro | |
|---|---|---|---|---|---|---|
| | | | | | modulation | |
| ChemBridge | | Docking | | | Steady- | Non-Steady |
| ID | | score | H-bond | Rec_Buried | state | state |
| 31590066* | 4FVT | −9.9 | R158, E177 | 0.77 | 92.6 | 103.3 |
| (FIG. 128) | | | | | | |
| 7693862* | | −10.1 | R158, E323 | 0.90 | 106.7 | 108.1 |
| (FIG. 15) | | | | | | |
| 40607037 | | −9.6 | D156, R158 | 0.74 | 78.4 | 77.2 |
| 5749820* | | −9.7 | E177 | 0.82 | 65.8 | 103.0 |
| 55411721 | 4BVG | −10.2 | A146, I154, Y204, I230, D231 | 0.85 | 72.4 | 75.9 |
| 6068318* | | −9.8 | P155, N229, Y204 | 0.86 | 103.1 | 112.6 |
| (FIG. 12) | | | | | | |
| 9143859 | | −9.9 | P155, Y204, N229 | 0.91 | 94.0 | 97.8 |
| (FIG. 32) | | | | | | |
| 15395964 | | −10.5 | E198, Q228, N229 | 0.87 | 63.5 | 93.2 |
| 97954491 | | −10.6 | Q228, N229, Y204 | 0.89 | 47.2 | 86.0 |
| 98284595 | | −9.7 | E198, Q228, N229 | 0.84 | 41.6 | 94.7 |
| 17946256* | | −9.9 | Y204, I230, D231 | 0.95 | 98.2 | 103.1 |
| (FIG. 227) | | | | | | |
| 7389387 | | −9.7 | Y204, N229 | 0.87 | 92.8 | 95.6 |
| 7965907* | | −9.9 | P155, I230, D231 | 0.87 | 96.9 | 106.1 |
| (FIG. 31) | | | | | | |
| 88832064 | | −9.7 | P155, Y204 | 0.89 | 69.5 | 94.6 |
| 36344000 | | −9.6 | E198, I230 | 0.93 | 92.1 | 94.6 |
| 94672147 | | −9.6 | Y204, N229 | 0.85 | 59.6 | 96.1 |
| 24556827* | | −9.6 | P155, Y204, I230 | 0.88 | 86.8 | 104.2 |
| (FIG. 97) | | | | | | |
| 6875218* | Over | 4FVT: −9.8 | 4FVT: None | 4FVT: 0.84 | 91.3 | 114.0 |
| (FIG. 13) | | 4BVG: −9.6 | 4BVG: P155, Y204 | 4BVG: 0.87 | | |
| 6318204 | | 4FVT: −9.7 | 4FVT: R158, D156 | 4FVT: 0.85 | 96.3 | 101.6 |
| | | 4BVG: −9.7 | 4BVG: None | 4BVG: 0.82 | | |
| 5761865* | | 4FVT: −9.6 | 4FVT: R158 | 4FVT: 0.87 | 52.7 | 103.1 |
| | | 4BVG: −9.8 | 4BVG: None | 4BVG: 0.85 | | |

### Comparison of Binding Mode of Top Hit Compounds to HKL

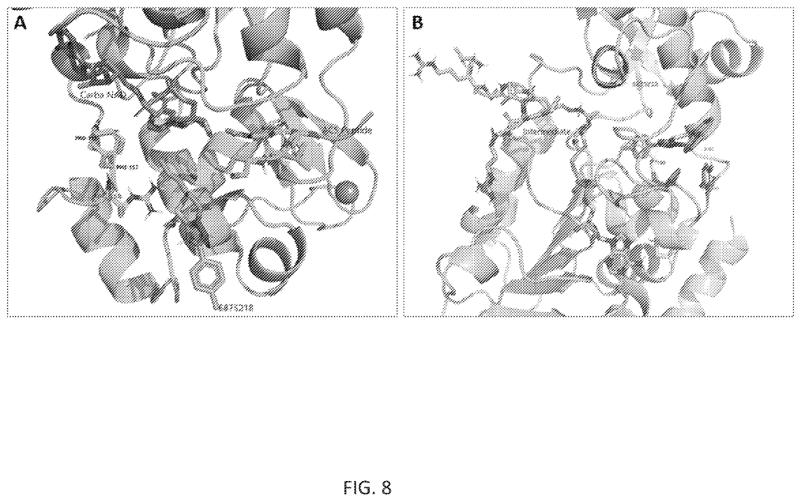

The interaction of residues in binding site which participate in the docking, esp. flexible loop, was analyzed for the top hit compounds (see Table 4). The top hit compounds had better AutoDock scores than that of reference ligand (HKL). Pro155, Arg158, and Ile230 interact with top hit compounds through hydrogen bond, in a pattern similar to HKL. Our previous study had shown that HKL forms hydrogen bond with Pro155 in SIRT3 open loop conformation and Arg158 in SIRT3 close loop conformation. Interestingly, the hit compounds 6068318 and 5761865 have the same binding pattern (FIG. 8).

### Validation of Selected Hit Compounds for SIRT3 Modulation Effect

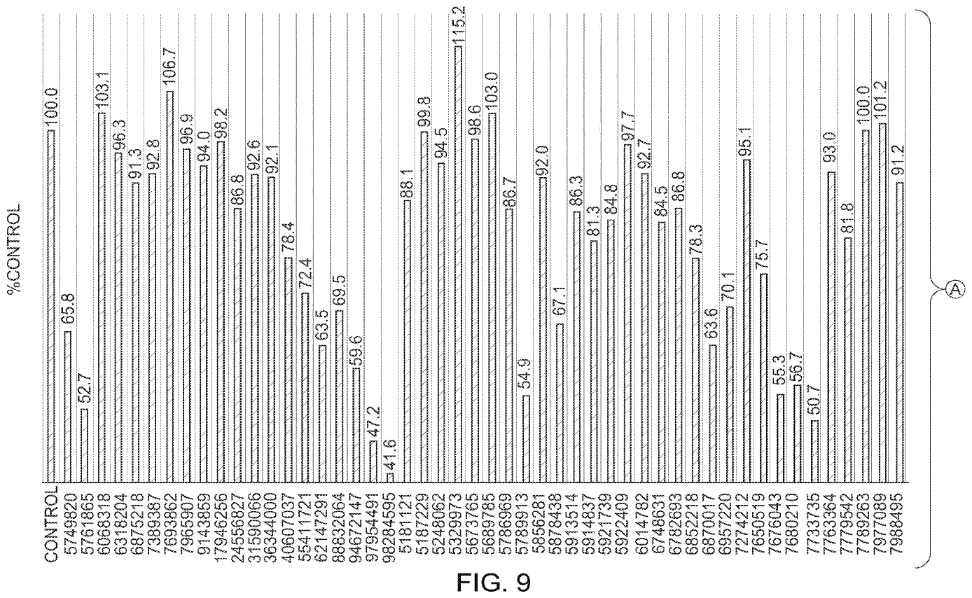

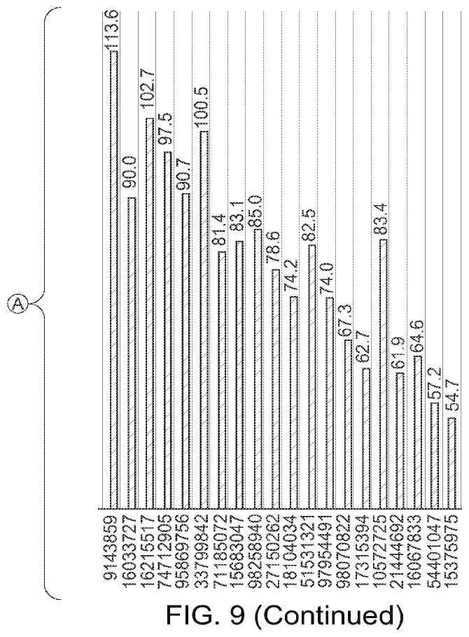

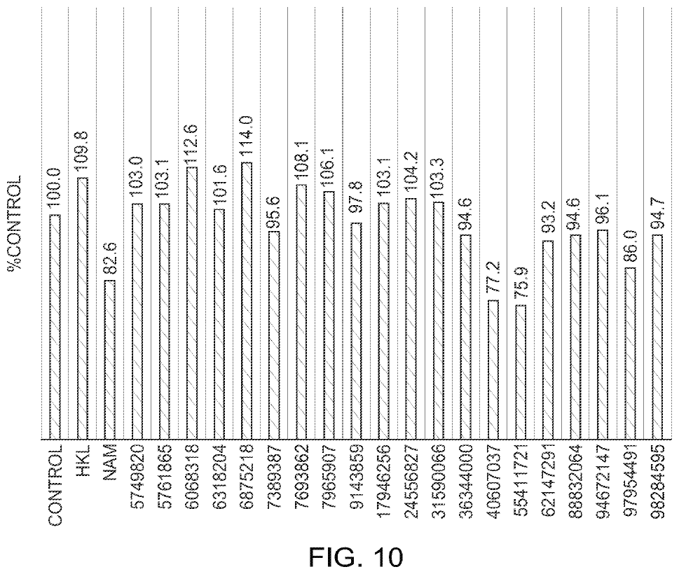

The in vitro validation of the hit compounds was performed with a commercial Fluor de Lys enzymatic assay and HPLC assay under steady state condition (top 70) and non-steady state (top 20) in FIGS. 9 and 10. respectively.

The compounds showed inhibition potency against SIRT3 in the range 0-58.4% at 10 μM modulator concentration (Table 3) under steady state condition. 2 hit compounds have shown good inhibition potency (>50% inhibition) in 10 μM modulator concentration. 9 out of 20 compounds were found activating SIRT3 (3.1%-14.0%) in 10 μM modulator concentration under non-steady state condition. The hit rate (the number of hits divided by the number of tested compounds) for VS is 45% indicating the current VS workflow is successful.

Comparison of Top Hit Compounds with Respect to the Binding Mode of HKL

The residues that participate in the docking especially the flexible loop area were analyzed for the top hit compounds (Table 4). The top hit compounds have better AutoDock scores than that of reference ligand (HKL). Pro155, Arg158, and Ile230 were found to interact with top hit compounds through hydrogen bond, which is similar to that HKL does. Our previous study had shown that HKL form hydrogen bond with Pro155 in SIRT3 open loop conformation and Arg158 in SIRT3 close loop conformation. Interestingly the hit compounds 6068318 and 5761865 present the same binding features. The definition of the docking site, docking template, and ligand query play an important role to ensure the high hit rate (45% for activators and 55% for inhibitors and activators) of current VS.

### Structural Similarity of Top Hit Compounds

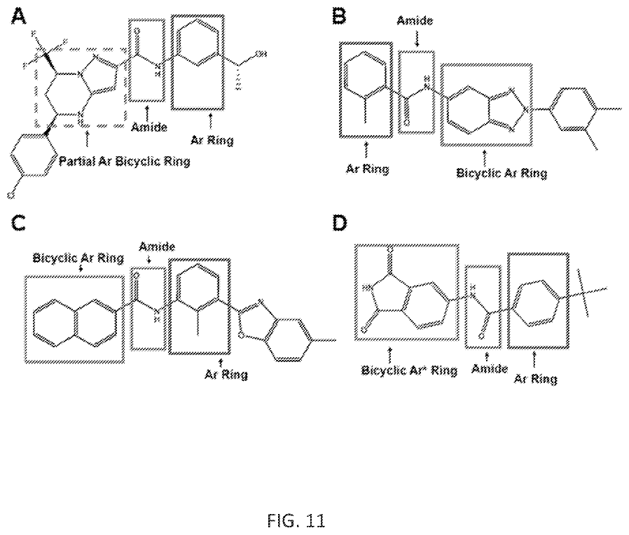

While searching for analogs of the top 9 assay-validated hits from our 1.2 million compound docking study, with two SIRT3 receptor structures (4BVG and 4FVT), it became known that some hits have common structural features. These common features are not sufficient for construction of a proper SAR model but are adequate to influence choice of building blocks for combinatorial libraries. The most obvious recurring motif is a bicyclic ring (aromatic, partially aromatic or with partial delocalization) connected to another solitary aromatic ring via an amide (or urea) linker (FIG. 11). These criteria can be applied when designing the scaffold of future ligands and combinatorial libraries.

Compounds of Formulas III, V, VI, VII, VIII, IX, X, XI, and XIV were included in a SIRT3 docking study as follows (tables 6-32). Initial 3D structures of the compounds were generated from SMILES strings using RDKit (2019 version), or, if this failed, Openbabel version 3.0. In cases of ambiguity (axial vs equatorial substitution for example), all conformers were generated. In addition, all stereoisomers were generated for racemates. For structures with non-standard ring puckers, structures were minimised using DFT to ensure feasible geometries were used for docking. This was carried out using ORCA version 3.0.1. GOLD v 5.2 was used to dock all compounds into SIRT3 using 3 PDB files, 4BVG and 4FVT, along with an internally-generated xray structure of human SIRT3 (called Xtal). In each case the binding pocket was restricted to anything within 20 Å of the ligand in the original PDB file, and 30 docks were requested for each ligand. All other options were set to the default options. An in-house algorithm was used to identify and assess the quality of all hydrogen bonds (H-bonds) made between ligand and protein and this information was used along with the GOLD docking score to select optimal solutions. Two docks were selected for each compound, namely the highest-scoring dock that contained the most H-bonds, and the highest scoring of the remaining docks. These docks were manually assessed and compared with a parent reference compound used to seed the design.

In the following tables of docking results, the highest-scoring dock independently of the number of hydrogen bonds was chosen. Compounds with a docking score at least higher than 90% of the docking score (GoldScore) of the reference compounds have been considered as a potential SIRT3 modulator.

Reference compounds on which docking studies are based are labeled as such in tables 6-32. In fact, these are compounds from the Virtual Screening top 20, which have been involved in laboratory activity tests and shown to modulate sirtuin. Results of Activity Tests of the Reference compounds are gathered in the table 5, values given are based on tests carried out in triplicate.

TABLE 5
Results of Activity Tests on Reference compounds (from Top 20 VS).

| | | Conditions I | | | Conditions II | |
|---|---|---|---|---|---|---|
| | | HPLC | FdL | Effect | HPLC | Effect |
| FIG. 12 | Formula III | 103.1 | 100.2 | Slight Activator | 112.6 | Activator |
| FIG. 13 | Formula III | 91.3 | 60 | Strong Inhibitor | 114.0 | Activator |
| FIG. 14 | Formula III | 106.7 | 101.2 | Slight Activator | 108.1 | Activator |
| FIG. 31 | Formula VI | 96.9 | ND | Inhibitor | 106.1 | Activator |
| FIG. 32 | Formula VI | 113.6 | ND | Activator | ND | |
| FIG. 43 | Formula V | 55.3 | ND | Strong Inhibitor | ND | |
| FIG. 44 | Formula V | 50.7 | ND | Strong Inhibitor | ND | |
| FIG. 45 | Formula V | 101.2 | ND | Slight Activator | ND | |
| FIG. 66 | Formula VII | 115.2 | ND | Activator | ND | |
| FIG. 97 | Formula VIII | 86.8 | 97.6 | Inhibitor | 104.2 | Slight Activator |
| FIG. 128 | Formula IX | 92.6 | 92.5 | Inhibitor | 103.3 | Slight Activator |
| FIG. 168 | Formula X | 102.7 | ND | Slight Activator | ND | |
| FIG. 199 | Formula XI | 113.6 | ND | Activator | ND | |
| FIG. 227 | Formula XIV | 98.2 | 88.1 | Inhibitor | 103.1 | Slight Activator |

Results of the docking studies employing Compounds of Formulas III, V-XI, and XIV are provided in Tables 6-32 below.

TABLE 6
Compounds of Formula III SIRT3 Docking Results
4 FVT Model

| Compound | Nb of H-Bond | Best Docking Score |
|---|---|---|
| FIG. 12 (Reference) | 0 | 71.26 |
| FIG. 13 (Reference) | 0 | 61.78 |
| FIG. 14 (Reference) | 1 | 75.32 |
| FIG. 15 | 0 | 77.43 |
| FIG. 16 | 0 | 63.80 |
| FIG. 17 | 0 | 70.88 |
| FIG. 18 | 1 | 58.61 |
| FIG. 19 | 1 | 57.48 |
| FIG. 20 | 1 | 78.05 |
| FIG. 21 | 1 | 78.75 |
| FIG. 22 | 0 | 79.34 |
| FIG. 23 | 3 | 72.37 |
| FIG. 24 | 2 | 57.89 |
| FIG. 25 | 3 | 87.82 |
| FIG. 26 | 1 | 71.09 |
| FIG. 27 | 2 | 86.88 |
| FIG. 28 | 2 | 91.44 |
| FIG. 29 | 1 | 74.58 |
| FIG. 30 | 0 | 62.24 |

TABLE 7
Compounds of Formula III SIRT3 Docking Results
4 BVG Model

| Compound | Nb of H-Bond | Best Docking Score |
|---|---|---|
| FIG. 12 (Reference) | 3 | 74.84 |
| FIG. 13 (Reference) | 0 | 68.03 |
| FIG. 14 (Reference) | 2 | 89.39 |
| FIG. 15 | 0 | 87.73 |
| FIG. 17 | 0 | 63.43 |
| FIG. 20 | 3 | 82.93 |
| FIG. 21 | 3 | 86.66 |
| FIG. 22 | 3 | 87.10 |
| FIG. 23 | 2 | 72.40 |
| FIG. 24 | 3 | 62.83 |
| FIG. 25 | 2 | 83.91 |
| FIG. 26 | 2 | 68.12 |
| FIG. 27 | 1 | 78.49 |
| FIG. 28 | 1 | 88.45 |
| FIG. 29 | 2 | 70.16 |

TABLE 8
Compounds of Formula III SIRT3 Docking Results
Xtal Model

| Compound | Nb of H-Bond | Best Docking Score |
|---|---|---|
| FIG. 12 (Reference) | 2 | 85.32 |
| FIG. 13 (Reference) | 0 | 74.49 |
| FIG. 14 (Reference) | 2 | 94.40 |
| FIG. 15 | 0 | 94.65 |
| FIG. 16 | 1 | 74.33 |
| FIG. 17 | 1 | 67.17 |
| FIG. 20 | 2 | 93.70 |
| FIG. 21 | 2 | 90.53 |
| FIG. 22 | 2 | 98.19 |
| FIG. 23 | 4 | 79.39 |
| FIG. 24 | 0 | 70.27 |
| FIG. 25 | 2 | 89.69 |
| FIG. 26 | 2 | 79.51 |
| FIG. 27 | 2 | 92.96 |
| FIG. 28 | 2 | 97.85 |
| FIG. 29 | 3 | 79.38 |

TABLE 9
Compounds of Formula VI SIRT3 Docking Results
4 FVT Model

| Compound | Nb of H-Bond | Best Docking Score |
|---|---|---|
| FIG. 31 (Reference) | 1 | 48.65 |
| FIG. 32 (Reference) | 3 | 64.89 |
| FIG. 33 | 1 | 62.45 |
| FIG. 34 | 1 | 63.99 |
| FIG. 35 | 1 | 77.44 |
| FIG. 36 | 2 | 58.80 |
| FIG. 37 | 0 | 61.85 |
| FIG. 38 | 1 | 60.70 |
| FIG. 39 | 0 | 60.75 |
| FIG. 40 | 0 | 60.98 |
| FIG. 41 | 0 | 52.72 |
| FIG. 42 | 1 | 62.16 |

TABLE 10
Compounds of Formula VI SIRT3 Docking Results
4 BVG Model

| Compound | Nb of H-Bond | Best Docking Score |
|---|---|---|
| FIG. 31 (Reference) | 0 | 56.28 |
| FIG. 32 (Reference) | 0 | 56.72 |
| FIG. 33 | 2 | 67.98 |
| FIG. 34 | 1 | 60.15 |
| FIG. 35 | 1 | 60.68 |
| FIG. 36 | 1 | 51.22 |
| FIG. 37 | 0 | 55.00 |
| FIG. 38 | 0 | 51.57 |
| FIG. 39 | 1 | 53.80 |
| FIG. 40 | 1 | 62.61 |
| FIG. 41 | 0 | 55.01 |
| FIG. 42 | 0 | 55.61 |

TABLE 11
Compounds of Formula VI SIRT3 Docking Results
Xtal Model

| Compound | Nb of H-Bond | Best Docking Score |
|---|---|---|
| FIG. 31 (Reference) | 0 | 58.60 |
| FIG. 32 (Reference) | 3 | 54.94 |
| FIG. 33 | 0 | 68.83 |
| FIG. 34 | 1 | 59.80 |
| FIG. 35 | 1 | 66.77 |
| FIG. 36 | 0 | 59.86 |
| FIG. 37 | 4 | 56.59 |
| FIG. 38 | 2 | 55.00 |
| FIG. 39 | 1 | 58.30 |
| FIG. 40 | 0 | 71.52 |
| FIG. 41 | 0 | 58.73 |
| FIG. 42 | 0 | 61.31 |

TABLE 12
Compounds of Formula V SIRT3 Docking Results
4 FVT Model

| Compound | Nb of H-Bond | Best Docking Score |
|---|---|---|
| FIG. 43 (Reference) | 1 | 77.32 |
| FIG. 44 (Reference) | 0 | 72.45 |
| FIG. 45 (Reference) | 0 | 69.94 |
| FIG. 46 | 0 | 70.36 |
| FIG. 47 | 1 | 66.87 |
| FIG. 48 | 2 | 68.84 |
| FIG. 49 | 1 | 70.84 |
| FIG. 50 | 1 | 71.57 |
| FIG. 51 | 1 | 66.32 |
| FIG. 52 | 1 | 67.29 |
| FIG. 53 | 0 | 67.01 |
| FIG. 54 | 1 | 70.65 |
| FIG. 55 | 1 | 65.30 |
| FIG. 56 | 0 | 78.29 |
| FIG. 57 | 3 | 74.64 |
| FIG. 58 | 1 | 75.29 |
| FIG. 59 | 1 | 75.22 |
| FIG. 60 | 1 | 67.10 |
| FIG. 61 | 0 | 77.79 |
| FIG. 62 | 0 | 73.21 |
| FIG. 63 | 0 | 77.47 |
| FIG. 64 | 0 | 79.06 |
| FIG. 65 | 0 | 76.21 |

TABLE 13
Compounds of Formula V SIRT3 Docking Results
4 BVG Model

| Compound | Nb of H-Bond | Best Docking Score |
|---|---|---|
| FIG. 43 (Reference) | 0 | 71.65 |
| FIG. 44 (Reference) | 0 | 72.60 |
| FIG. 45 (Reference) | 2 | 68.45 |
| FIG. 46 | 0 | 63.84 |
| FIG. 47 | 2 | 65.57 |
| FIG. 48 | 1 | 66.34 |
| FIG. 49 | 0 | 71.05 |
| FIG. 50 | 1 | 66.21 |
| FIG. 51 | 1 | 74.18 |
| FIG. 52 | 1 | 69.34 |
| FIG. 53 | 0 | 65.72 |
| FIG. 55 | 1 | 64.39 |
| FIG. 56 | 1 | 75.76 |
| FIG. 57 | 1 | 69.09 |
| FIG. 58 | 1 | 75.94 |
| FIG. 59 | 2 | 83.48 |
| FIG. 60 | 0 | 72.46 |
| FIG. 61 | 0 | 80.59 |
| FIG. 62 | 0 | 79.06 |
| FIG. 63 | 0 | 76.03 |
| FIG. 64 | 2 | 77.54 |
| FIG. 65 | 1 | 71.28 |

TABLE 14
Compounds of Formula V SIRT3 Docking Results
Xtal Model

| Compound | Nb of H-Bond | Best Docking Score |
|---|---|---|
| FIG. 43 (Reference) | 3 | 73.18 |
| FIG. 44 (Reference) | 0 | 82.80 |
| FIG. 45 (Reference) | 1 | 82.29 |
| FIG. 46 | 1 | 68.49 |
| FIG. 47 | 2 | 86.68 |
| FIG. 48 | 0 | 71.20 |
| FIG. 49 | 1 | 85.42 |
| FIG. 50 | 2 | 73.35 |
| FIG. 51 | 0 | 76.33 |
| FIG. 52 | 1 | 76.35 |
| FIG. 53 | 0 | 69.30 |
| FIG. 54 | 1 | 76.02 |
| FIG. 55 | 2 | 69.65 |
| FIG. 56 | 1 | 85.08 |
| FIG. 57 | 1 | 83.98 |
| FIG. 58 | 1 | 85.14 |
| FIG. 59 | 0 | 79.51 |
| FIG. 60 | 2 | 73.16 |
| FIG. 61 | 0 | 87.91 |
| FIG. 62 | 0 | 86.89 |
| FIG. 63 | 0 | 84.47 |
| FIG. 64 | 0 | 88.25 |
| FIG. 65 | 0 | 84.96 |

TABLE 15
Compounds of Formula VII SIRT3 Docking Results
4 FVT Model

| Compound | Nb of H-Bond | Best Docking Score |
|---|---|---|
| FIG. 66 (Reference) | 2 | 55.00 |
| FIG. 67 | 1 | 62.34 |
| FIG. 68 | 2 | 60.35 |
| FIG. 69 | 0 | 61.98 |
| FIG. 70 | 3 | 56.70 |
| FIG. 71 | 0 | 65.06 |
| FIG. 72 | 0 | 59.34 |
| FIG. 73 | 3 | 61.78 |
| FIG. 74 | 3 | 67.14 |
| FIG. 75 | 0 | 60.48 |
| FIG. 76 | 3 | 60.53 |
| FIG. 77 | 0 | 57.52 |
| FIG. 78 | 1 | 52.37 |
| FIG. 79 | 0 | 60.40 |
| FIG. 80 | 2 | 53.93 |
| FIG. 81 | 2 | 56.50 |
| FIG. 82 | 0 | 73.91 |
| FIG. 83 | 3 | 54.41 |
| FIG. 84 | 2 | 59.42 |
| FIG. 85 | 0 | 65.87 |
| FIG. 86 | 0 | 62.76 |
| FIG. 87 | 0 | 58.45 |
| FIG. 88 | 2 | 60.12 |
| FIG. 89 | 2 | 58.58 |
| FIG. 90 | 0 | 55.74 |
| FIG. 91 | 0 | 60.04 |
| FIG. 92 | 0 | 62.91 |
| FIG. 93 | 0 | 62.61 |
| FIG. 94 | 0 | 63.70 |
| FIG. 95 | 1 | 56.56 |
| FIG. 96 | 2 | 56.95 |

TABLE 16
Compounds of Formula VII SIRT3 Docking Results
4 BVG Model

| Compound | Nb of H-Bond | Best Docking Score |
|---|---|---|
| FIG. 66 (Reference) | 1 | 62.00 |
| FIG. 67 | 1 | 62.27 |
| FIG. 68 | 0 | 63.47 |
| FIG. 69 | 2 | 67.88 |
| FIG. 70 | 1 | 64.09 |
| FIG. 73 | 0 | 61.33 |
| FIG. 74 | 1 | 68.82 |
| FIG. 75 | 0 | 63.41 |
| FIG. 76 | 2 | 65.16 |
| FIG. 77 | 0 | 58.05 |
| FIG. 78 | 0 | 60.43 |
| FIG. 79 | 0 | 57.95 |
| FIG. 80 | 1 | 63.60 |
| FIG. 81 | 1 | 58.18 |
| FIG. 82 | 0 | 60.41 |
| FIG. 83 | 0 | 57.59 |
| FIG. 84 | 1 | 62.24 |
| FIG. 85 | 0 | 61.23 |
| FIG. 86 | 1 | 66.53 |
| FIG. 87 | 0 | 58.41 |
| FIG. 88 | 1 | 66.38 |
| FIG. 89 | 0 | 61.52 |
| FIG. 90 | 2 | 63.28 |
| FIG. 92 | 0 | 60.40 |
| FIG. 93 | 1 | 62.26 |
| FIG. 94 | 1 | 61.61 |
| FIG. 95 | 0 | 61.34 |
| FIG. 96 | 0 | 58.12 |

TABLE 17
Compounds of Formula VII SIRT3 Docking Results
Xtal Model

| Compound | Nb of H-Bond | Best Docking Score |
|---|---|---|
| FIG. 66 (Reference) | 2 | 70.00 |
| FIG. 67 | 0 | 73.86 |
| FIG. 68 | 0 | 67.29 |
| FIG. 69 | 1 | 64.25 |
| FIG. 70 | 2 | 68.31 |
| FIG. 71 | 2 | 69.68 |
| FIG. 73 | 2 | 67.94 |
| FIG. 74 | 0 | 67.76 |
| FIG. 75 | 1 | 64.57 |
| FIG. 76 | 2 | 72.96 |
| FIG. 77 | 2 | 66.16 |
| FIG. 78 | 1 | 67.34 |
| FIG. 81 | 1 | 63.52 |
| FIG. 82 | 1 | 65.73 |
| FIG. 83 | 2 | 67.75 |
| FIG. 85 | 2 | 69.75 |
| FIG. 86 | 0 | 72.55 |
| FIG. 87 | 0 | 65.12 |
| FIG. 88 | 0 | 74.77 |
| FIG. 89 | 1 | 65.57 |
| FIG. 92 | 0 | 72.59 |
| FIG. 93 | 2 | 67.91 |
| FIG. 94 | 0 | 70.92 |
| FIG. 96 | 1 | 66.47 |

TABLE 18
Compounds of Formula VIII SIRT3 Docking Results
4 FVT Model

| Compound | Nb of H-Bond | Best Docking Score |
|---|---|---|
| FIG. 97 (Reference) | 2 | 68.00 |
| FIG. 98 | 0 | 67.97 |
| FIG. 99 | 1 | 61.80 |
| FIG. 100 | 2 | 74.18 |
| FIG. 101 | 2 | 74.74 |
| FIG. 102 | 2 | 72.72 |
| FIG. 103 | 2 | 71.42 |
| FIG. 104 | 1 | 74.07 |
| FIG. 105 | 0 | 68.95 |
| FIG. 106 | 3 | 69.90 |
| FIG. 107 | 1 | 64.11 |
| FIG. 108 | 2 | 70.87 |
| FIG. 109 | 2 | 69.06 |
| FIG. 110 | 2 | 70.63 |
| FIG. 111 | 0 | 62.99 |
| FIG. 112 | 2 | 70.35 |
| FIG. 113 | 2 | 69.15 |
| FIG. 114 | 2 | 71.78 |
| FIG. 115 | 2 | 76.80 |
| FIG. 116 | 2 | 77.11 |
| FIG. 117 | 1 | 70.98 |
| FIG. 118 | 1 | 67.67 |
| FIG. 119 | 2 | 68.11 |
| FIG. 120 | 3 | 74.16 |

TABLE 19
Compounds of Formula VIII SIRT3 Docking Results
4 BVG Model

| Compound | Nb of H-Bond | Best Docking Score |
|---|---|---|
| FIG. 97 (Reference) | 1 | 77.00 |
| FIG. 100 | 2 | 83.12 |
| FIG. 101 | 1 | 81.30 |
| FIG. 102 | 1 | 76.71 |
| FIG. 103 | 2 | 76.16 |
| FIG. 123 | 0 | 79.03 |
| FIG. 124 | 1 | 71.03 |
| FIG. 107 | 2 | 70.81 |
| FIG. 108 | 1 | 76.83 |
| FIG. 109 | 1 | 69.57 |
| FIG. 110 | 2 | 75.19 |
| FIG. 111 | 1 | 73.53 |
| FIG. 114 | 1 | 76.96 |
| FIG. 116 | 0 | 79.47 |
| FIG. 127 | 1 | 70.58 |
| FIG. 117 | 1 | 76.44 |
| FIG. 118 | 1 | 71.23 |

TABLE 20
Compounds of Formula VIII SIRT3 Docking Results
Xtal Model

| Compound | Nb of H-Bond | Best Docking Score |
|---|---|---|
| FIG. 97 (Reference) | 2 | 67.00 |
| FIG. 98 | 1 | 62.94 |
| FIG. 121 | 0 | 66.70 |
| FIG. 99 | 2 | 66.00 |
| FIG. 100 | 2 | 73.31 |
| FIG. 101 | 1 | 74.52 |
| FIG. 102 | 2 | 72.02 |
| FIG. 122 | 1 | 65.08 |
| FIG. 103 | 2 | 71.47 |
| FIG. 123 | 1 | 71.30 |
| FIG. 124 | 3 | 68.73 |
| FIG. 105 | 0 | 67.51 |
| FIG. 106 | 0 | 64.36 |
| FIG. 107 | 1 | 70.02 |
| FIG. 108 | 2 | 69.00 |
| FIG. 125 | 2 | 70.31 |
| FIG. 109 | 1 | 65.04 |
| FIG. 110 | 2 | 69.91 |
| FIG. 111 | 1 | 66.08 |
| FIG. 112 | 3 | 70.16 |
| FIG. 113 | 1 | 61.54 |
| FIG. 114 | 0 | 72.25 |
| FIG. 115 | 1 | 72.86 |
| FIG. 126 | 0 | 65.38 |
| FIG. 127 | 0 | 62.32 |
| FIG. 117 | 2 | 73.57 |
| FIG. 118 | 1 | 67.96 |
| FIG. 119 | 0 | 64.41 |
| FIG. 120 | 0 | 71.66 |

TABLE 21
Compounds of Formula IX SIRT3 Docking Results
4 FVT Model

| Compound | Nb of H-Bond | Best Docking Score |
|---|---|---|
| FIG. 128 (Reference) | 2 | 86.00 |
| FIG. 129 | 1 | 85.93 |
| FIG. 130 | 0 | 77.96 |
| FIG. 131 | 1 | 82.05 |
| FIG. 132 | 2 | 84.51 |
| FIG. 133 | 0 | 93.63 |
| FIG. 134 | 0 | 95.33 |
| FIG. 135 | 2 | 88.46 |
| FIG. 136 | 1 | 84.96 |
| FIG. 137 | 3 | 83.07 |
| FIG. 138 | 0 | 85.29 |
| FIG. 139 | 3 | 85.09 |
| FIG. 140 | 2 | 88.94 |
| FIG. 141 | 1 | 87.08 |
| FIG. 142 | 2 | 83.62 |
| FIG. 143 | 2 | 80.11 |
| FIG. 144 | 2 | 84.26 |

TABLE 22
Compounds of Formula IX SIRT3 Docking Results
4 BVG Model

| Compound | Nb of H-Bond | Best Docking Score |
|---|---|---|
| FIG. 128 (Reference) | 1 | 70.00 |
| FIG. 145 | 0 | 63.32 |
| FIG. 146 | 1 | 66.61 |
| FIG. 130 | 1 | 81.41 |
| FIG. 147 | 0 | 66.69 |
| FIG. 131 | 0 | 65.52 |
| FIG. 148 | 1 | 79.56 |
| FIG. 132 | 0 | 79.07 |
| FIG. 133 | 1 | 79.34 |
| FIG. 148 | 0 | 69.64 |
| FIG. 134 | 1 | 74.36 |
| FIG. 149 | 1 | 65.79 |
| FIG. 150 | 1 | 66.61 |
| FIG. 151 | 1 | 64.19 |
| FIG. 152 | 0 | 65.31 |
| FIG. 153 | 0 | 70.51 |
| FIG. 154 | 1 | 69.72 |
| FIG. 155 | 1 | 84.46 |
| FIG. 135 | 1 | 84.11 |
| FIG. 136 | 0 | 74.75 |
| FIG. 137 | 0 | 65.70 |
| FIG. 138 | 0 | 76.92 |
| FIG. 156 | 1 | 73.14 |
| FIG. 157 | 0 | 65.64 |
| FIG. 137 | 0 | 68.74 |
| FIG. 158 | 1 | 67.72 |
| FIG. 140 | 1 | 78.47 |
| FIG. 158 | 1 | 64.55 |
| FIG. 141 | 1 | 81.90 |
| FIG. 142 | 0 | 78.76 |
| FIG. 143 | 0 | 66.89 |
| FIG. 144 | 0 | 70.81 |
| FIG. 160 | 0 | 68.95 |

TABLE 23
Compounds of Formula IX SIRT3 Docking Results
Xtal Model

| Compound | Nb of H-Bond | Best Docking Score |
|---|---|---|
| FIG. 128 (Reference) | 1 | 74.00 |
| FIG. 161 | 0 | 67.39 |
| FIG. 162 | 2 | 68.77 |
| FIG. 163 | 1 | 67.47 |
| FIG. 130 | 2 | 79.65 |
| FIG. 147 | 0 | 73.93 |
| FIG. 131 | 1 | 72.74 |
| FIG. 148 | 2 | 77.39 |
| FIG. 132 | 0 | 73.82 |
| FIG. 133 | 0 | 84.26 |
| FIG. 148 | 0 | 74.01 |
| FIG. 134 | 0 | 71.51 |
| FIG. 164 | 2 | 68.03 |
| FIG. 150 | 2 | 74.96 |
| FIG. 151 | 1 | 68.84 |
| FIG. 152 | 0 | 70.95 |
| FIG. 153 | 0 | 76.07 |
| FIG. 165 | 1 | 77.41 |
| FIG. 166 | 0 | 69.46 |
| FIG. 155 | 1 | 79.29 |
| FIG. 135 | 0 | 83.18 |
| FIG. 136 | 1 | 78.59 |
| FIG. 137 | 0 | 67.28 |
| FIG. 138 | 1 | 79.20 |
| FIG. 157 | 1 | 68.26 |
| FIG. 139 | 1 | 77.95 |
| FIG. 140 | 1 | 99.46 |
| FIG. 141 | 1 | 94.80 |
| FIG. 167 | 1 | 66.91 |
| FIG. 142 | 1 | 73.43 |
| FIG. 143 | 0 | 75.27 |
| FIG. 144 | 1 | 75.92 |
| FIG. 160 | 0 | 74.82 |
| FIG. 159 | 2 | 66.84 |

TABLE 24
Compounds of Formula X SIRT3 Docking Results
4 FVT Model

| Compound | Nb of H-Bond | Best Docking Score |
|---|---|---|
| FIG. 168 (Reference) | 1 | 77.00 |
| FIG. 169 | 0 | 78.05 |
| FIG. 170 | 0 | 76.82 |
| FIG. 171 | 1 | 83.22 |
| FIG. 172 | 0 | 82.80 |
| FIG. 173 | 1 | 75.96 |
| FIG. 174 | 1 | 80.78 |
| FIG. 175 | 2 | 70.26 |
| FIG. 176 | 1 | 74.52 |
| FIG. 177 | 1 | 78.33 |
| FIG. 178 | 3 | 76.49 |
| FIG. 179 | 1 | 83.40 |
| FIG. 180 | 1 | 81.94 |
| FIG. 181 | 1 | 84.46 |
| FIG. 182 | 1 | 75.13 |
| FIG. 183 | 1 | 77.02 |
| FIG. 184 | 1 | 69.60 |
| FIG. 185 | 1 | 77.63 |
| FIG. 186 | 0 | 80.67 |
| FIG. 187 | 1 | 78.18 |
| FIG. 188 | 1 | 69.49 |
| FIG. 189 | 1 | 75.97 |
| FIG. 190 | 1 | 72.42 |
| FIG. 191 | 3 | 76.17 |
| FIG. 192 | 1 | 82.81 |

TABLE 25
Compounds of Formula X SIRT3 Docking Results
4 BVG Model

| Compound | Nb of H-Bond | Best Docking Score |
|---|---|---|
| FIG. 168 (Reference) | 1 | 68.00 |
| FIG. 169 | 1 | 67.48 |
| FIG. 170 | 1 | 72.81 |
| FIG. 193 | 0 | 65.04 |
| FIG. 171 | 0 | 75.62 |
| FIG. 172 | 1 | 76.44 |
| FIG. 173 | 0 | 65.16 |
| FIG. 174 | 0 | 71.37 |
| FIG. 175 | 3 | 63.12 |
| FIG. 176 | 0 | 65.94 |
| FIG. 177 | 1 | 72.36 |
| FIG. 178 | 1 | 76.28 |
| FIG. 179 | 0 | 67.96 |
| FIG. 194 | 0 | 65.89 |
| FIG. 180 | 1 | 72.22 |
| FIG. 195 | 0 | 66.95 |
| FIG. 181 | 1 | 76.08 |
| FIG. 182 | 0 | 75.05 |
| FIG. 183 | 1 | 70.84 |
| FIG. 184 | 0 | 70.91 |
| FIG. 185 | 0 | 71.15 |
| FIG. 196 | 1 | 65.37 |
| FIG. 186 | 1 | 74.27 |
| FIG. 187 | 1 | 71.47 |
| FIG. 197 | 1 | 68.54 |
| FIG. 190 | 1 | 66.49 |
| FIG. 191 | 1 | 70.45 |
| FIG. 198 | 0 | 66.32 |
| FIG. 192 | 2 | 69.90 |

TABLE 26
Compounds of Formula X SIRT3 Docking Results
Xtal Model

| Compound | Nb of H-Bond | Best Docking Score |
|---|---|---|
| FIG. 168 (Reference) | 1 | 82.00 |
| FIG. 169 | 2 | 87.52 |
| FIG. 170 | 0 | 78.67 |
| FIG. 193 | 1 | 77.81 |
| FIG. 171 | 0 | 82.86 |
| FIG. 172 | 1 | 96.49 |
| FIG. 173 | 2 | 78.19 |
| FIG. 174 | 1 | 89.50 |
| FIG. 176 | 1 | 84.75 |
| FIG. 177 | 0 | 92.27 |
| FIG. 178 | 0 | 88.04 |
| FIG. 179 | 1 | 84.74 |
| FIG. 194 | 1 | 74.22 |
| FIG. 180 | 0 | 86.10 |
| FIG. 195 | 1 | 79.63 |
| FIG. 181 | 0 | 88.24 |
| FIG. 182 | 1 | 84.10 |
| FIG. 183 | 0 | 76.29 |
| FIG. 185 | 1 | 88.36 |
| FIG. 196 | 3 | 74.62 |
| FIG. 186 | 1 | 83.43 |
| FIG. 187 | 0 | 89.61 |
| FIG. 188 | 0 | 75.79 |
| FIG. 197 | 1 | 81.39 |
| FIG. 190 | 1 | 78.37 |
| FIG. 192 | 1 | 87.64 |

TABLE 27
Compounds of Formula XI SIRT3 Docking Results
4 FVT Model

| Compound | Nb of H-Bond | Best Docking Score |
|---|---|---|
| FIG. 199 (Reference) | 1 | 65.00 |
| FIG. 200 | 0 | 72.58 |
| FIG. 201 | 1 | 65.45 |
| FIG. 202 | 0 | 71.43 |
| FIG. 203 | 1 | 56.65 |
| FIG. 204 | 0 | 64.97 |
| FIG. 205 | 1 | 64.72 |
| FIG. 206 | 0 | 70.29 |
| FIG. 207 | 2 | 76.28 |
| FIG. 208 | 0 | 64.31 |
| FIG. 209 | 1 | 71.96 |
| FIG. 210 | 1 | 75.81 |
| FIG. 211 | 0 | 71.11 |
| FIG. 212 | 0 | 61.71 |
| FIG. 213 | 0 | 65.17 |
| FIG. 214 | 2 | 68.83 |
| FIG. 215 | 0 | 61.54 |
| FIG. 216 | 0 | 65.75 |
| FIG. 217 | 3 | 64.90 |
| FIG. 218 | 0 | 60.47 |
| FIG. 219 | 2 | 74.67 |
| FIG. 220 | 0 | 61.16 |

TABLE 28
Compounds of Formula XI SIRT3 Docking Results
4 BVG Model

| Compound | Nb of H-Bond | Best Docking Score |
|---|---|---|
| FIG. 199 (Reference) | 2 | 68.00 |
| FIG. 200 | 1 | 74.71 |
| FIG. 201 | 0 | 71.34 |
| FIG. 202 | 0 | 64.02 |
| FIG. 203 | 1 | 69.42 |
| FIG. 204 | 1 | 69.08 |
| FIG. 205 | 1 | 68.12 |
| FIG. 206 | 1 | 64.33 |
| FIG. 207 | 0 | 78.91 |
| FIG. 208 | 1 | 69.51 |
| FIG. 209 | 0 | 69.48 |
| FIG. 210 | 1 | 73.83 |
| FIG. 211 | 0 | 76.69 |
| FIG. 212 | 0 | 70.98 |
| FIG. 213 | 2 | 71.00 |
| FIG. 214 | 1 | 66.34 |
| FIG. 215 | 0 | 69.79 |
| FIG. 216 | 1 | 70.92 |
| FIG. 217 | 1 | 64.31 |
| FIG. 221 | 1 | 61.45 |
| FIG. 222 | 0 | 65.52 |
| FIG. 223 | 0 | 70.47 |
| FIG. 219 | 0 | 79.58 |

TABLE 29
Compounds of Formula XI SIRT3 Docking Results
Xtal Model

| Compound | Nb of H-Bond | Best Docking Score |
|---|---|---|
| FIG. 199 (Reference) | 1 | 70.00 |
| FIG. 200 | 1 | 81.84 |
| FIG. 201 | 0 | 67.96 |
| FIG. 202 | 0 | 74.84 |
| FIG. 203 | 0 | 79.74 |
| FIG. 204 | 1 | 71.95 |
| FIG. 205 | 0 | 86.84 |
| FIG. 206 | 0 | 72.04 |
| FIG. 207 | 1 | 90.16 |
| FIG. 208 | 3 | 73.40 |
| FIG. 209 | 0 | 80.13 |
| FIG. 211 | 1 | 85.00 |
| FIG. 212 | 0 | 77.25 |
| FIG. 213 | 1 | 71.36 |
| FIG. 214 | 0 | 70.29 |
| FIG. 215 | 0 | 66.97 |
| FIG. 216 | 1 | 78.28 |
| FIG. 217 | 3 | 63.99 |
| FIG. 224 | 1 | 66.29 |
| FIG. 221 | 2 | 68.53 |
| FIG. 225 | 0 | 69.13 |
| FIG. 222 | 1 | 63.78 |
| FIG. 223 | 0 | 76.99 |
| FIG. 218 | 1 | 69.13 |
| FIG. 219 | 0 | 93.22 |
| FIG. 226 | 2 | 70.93 |
| FIG. 220 | 1 | 71.11 |

TABLE 30
Compounds of Formula XIV SIRT3 Docking Results
4 FVT Model

| Compound | Nb of H-Bond | Best Docking Score |
|---|---|---|
| FIG. 227 (Reference) | 0 | 70.75 |
| FIG. 228 | 1 | 69.21 |
| FIG. 229 | 1 | 72.72 |
| FIG. 230 | 1 | 74.53 |
| FIG. 231 | 1 | 65.97 |
| FIG. 232 | 3 | 72.64 |
| FIG. 233 | 3 | 76.72 |
| FIG. 234 | 1 | 67.85 |
| FIG. 235 | 1 | 68.29 |
| FIG. 236 | 1 | 64.01 |
| FIG. 237 | 1 | 64.41 |
| FIG. 238 | 2 | 71.08 |
| FIG. 239 | 1 | 66.88 |
| FIG. 240 | 3 | 70.98 |
| FIG. 241 | 3 | 66.51 |
| FIG. 242 | 1 | 66.13 |
| FIG. 243 | 1 | 72.53 |
| FIG. 244 | 3 | 64.22 |
| FIG. 245 | 3 | 63.77 |

TABLE 31
Compounds of Formula XIV SIRT3 Docking Results
4 BVG Model

| Compound | Nb of H-Bond | Best Docking Score |
|---|---|---|
| FIG. 227 (Reference) | 1 | 67.85 |
| FIG. 228 | 0 | 62.84 |
| FIG. 229 | 1 | 69.84 |
| FIG. 230 | 3 | 75.32 |
| FIG. 231 | 1 | 65.88 |
| FIG. 232 | 2 | 71.53 |
| FIG. 233 | 2 | 70.59 |
| FIG. 234 | 1 | 65.32 |
| FIG. 235 | 1 | 70.47 |
| FIG. 236 | 1 | 66.94 |
| FIG. 237 | 0 | 67.44 |
| FIG. 238 | 2 | 75.21 |
| FIG. 239 | 0 | 64.85 |
| FIG. 240 | 1 | 68.76 |
| FIG. 241 | 1 | 67.44 |
| FIG. 244 | 2 | 68.44 |
| FIG. 245 | 1 | 70.11 |
| FIG. 246 | 1 | 63.90 |
| FIG. 247 | 1 | 62.69 |
| FIG. 248 | 2 | 67.46 |
| FIG. 249 | 1 | 65.59 |
| FIG. 250 | 2 | 61.10 |
| FIG. 251 | 3 | 61.82 |
| FIG. 252 | 2 | 67.97 |
| FIG. 253 | 1 | 66.02 |
| FIG. 254 | 1 | 62.56 |

TABLE 32
Compounds of Formula XIV SIRT3 Docking Results
Xtal Model

| Compound | Nb of H-Bond | Best Docking Score |
|---|---|---|
| FIG. 227 (Reference) | 1 | 67.99 |
| FIG. 228 | 0 | 66.08 |
| FIG. 229 | 0 | 67.48 |
| FIG. 230 | 0 | 75.22 |
| FIG. 231 | 1 | 66.08 |
| FIG. 232 | 0 | 68.20 |
| FIG. 233 | 1 | 74.49 |
| FIG. 234 | 1 | 69.01 |
| FIG. 235 | 1 | 67.72 |
| FIG. 236 | 0 | 63.99 |
| FIG. 237 | 0 | 77.75 |
| FIG. 238 | 0 | 66.57 |
| FIG. 239 | 1 | 67.53 |
| FIG. 240 | 1 | 77.53 |
| FIG. 242 | 0 | 68.77 |
| FIG. 243 | 1 | 67.79 |
| FIG. 245 | 2 | 64.93 |
| FIG. 247 | 1 | 63.40 |
| FIG. 249 | 2 | 69.11 |
| FIG. 255 | 0 | 73.74 |
| FIG. 256 | 1 | 67.60 |
| FIG. 257 | 1 | 63.14 |
| FIG. 258 | 1 | 65.01 |
| FIG. 259 | 0 | 62.62 |

The docking results show that the analogues have docking scores very close to or lower than the docking scores of the reference compounds (whose modulation—activation or inhibition—of SIRT3 has been identified by activity tests carried out in the laboratory). This suggests that these analogues also interact with the SIRT3 protein in a similar way, modulating its activity.

Additional compounds falling under Formulas III, V, VII and XI were tested for their activity in Conditions I (Steady State). These activity tests showed that these compounds modulated SIRT3 activity (activation or inhibition), confirming the results obtained from the docking studies. Table 33 sums up the results of these activity tests.

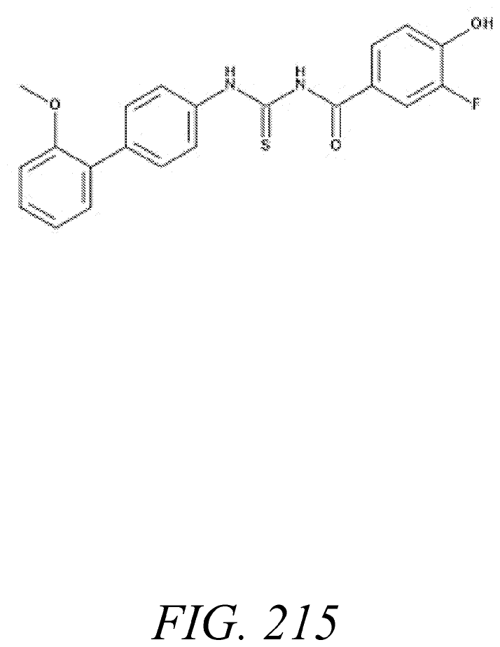

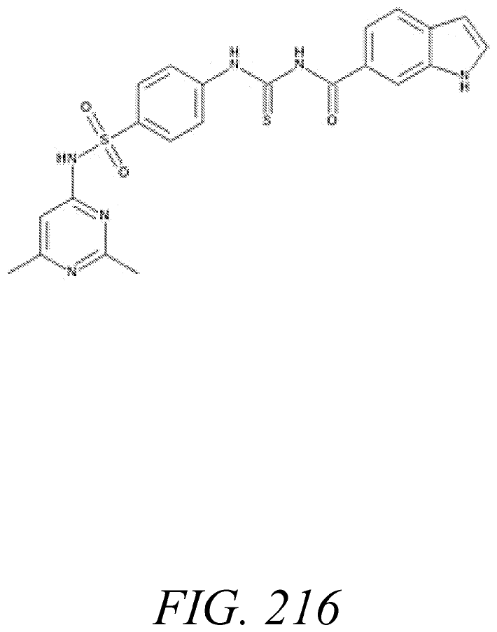

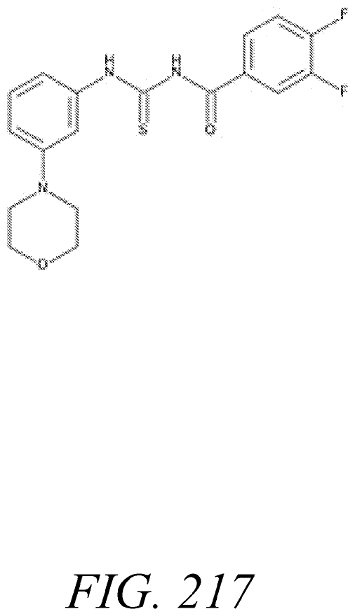

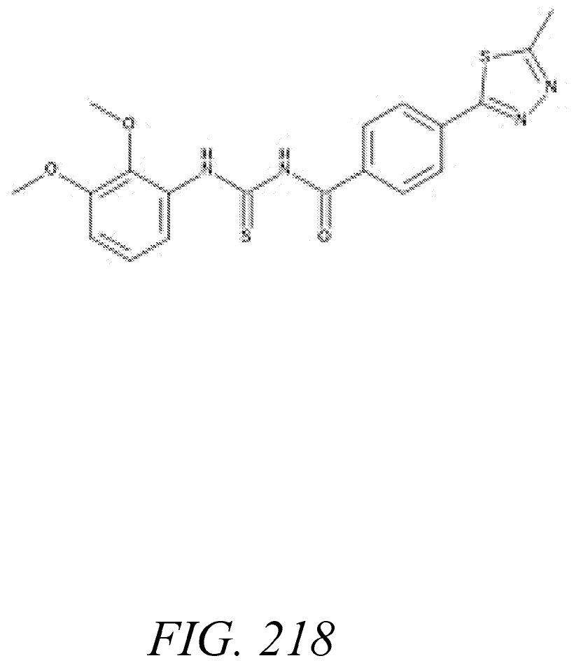

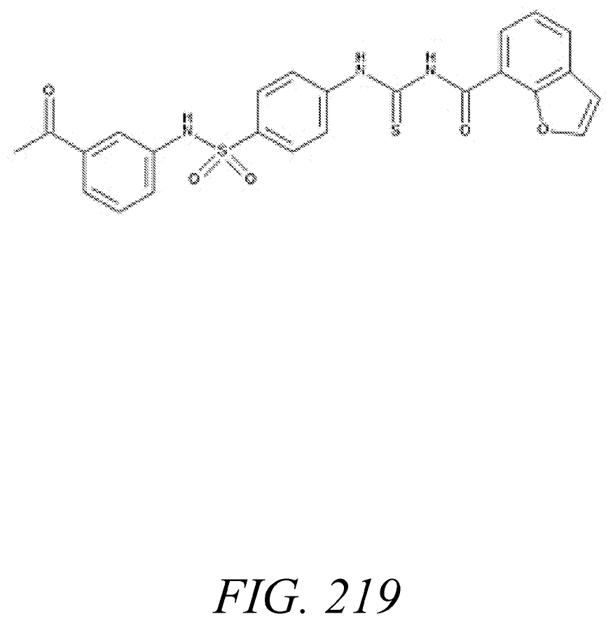

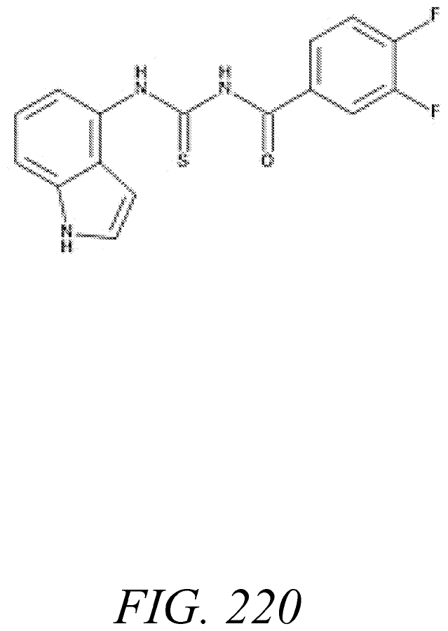

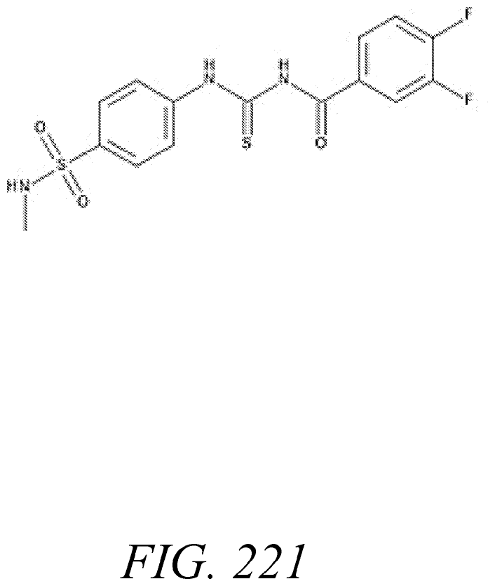

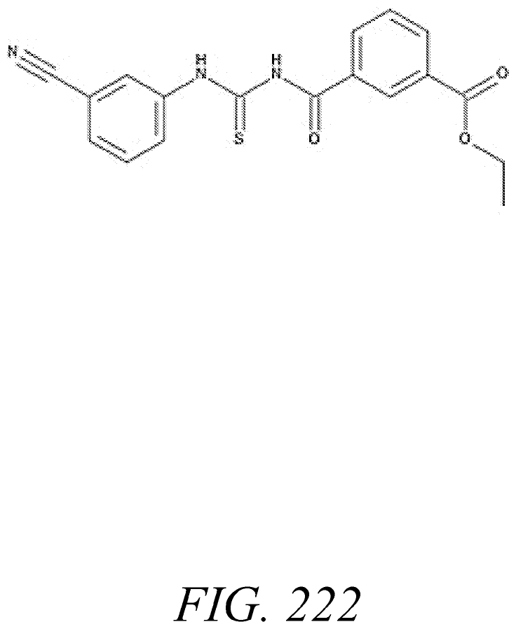

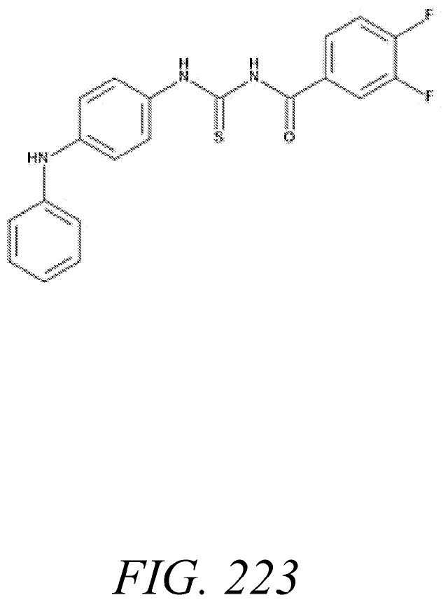

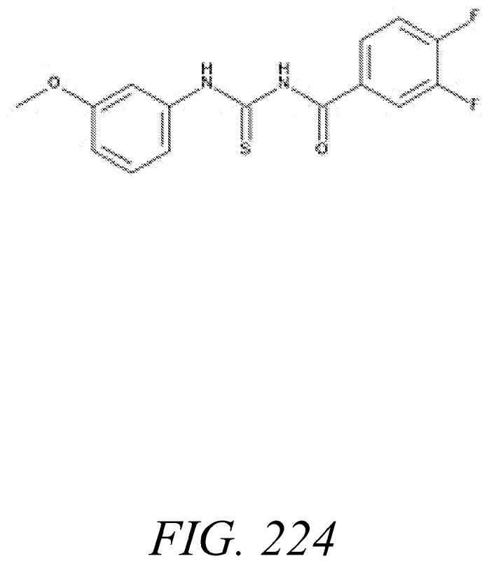

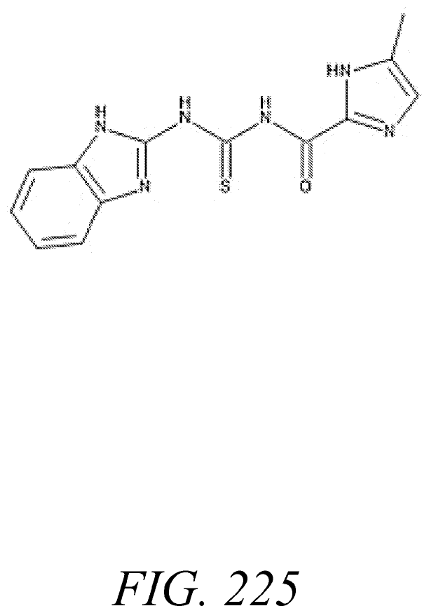

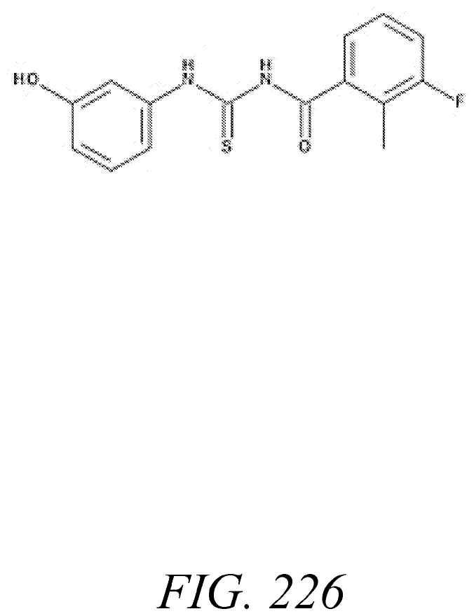

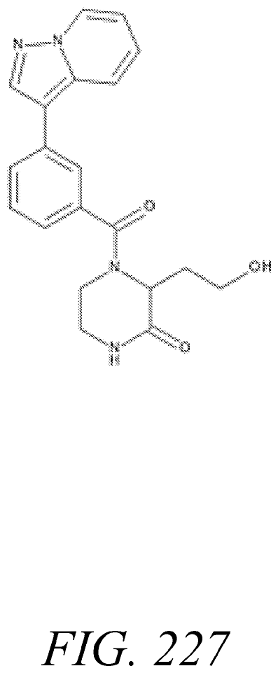

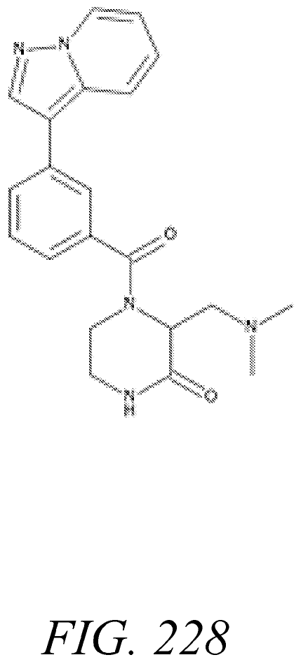

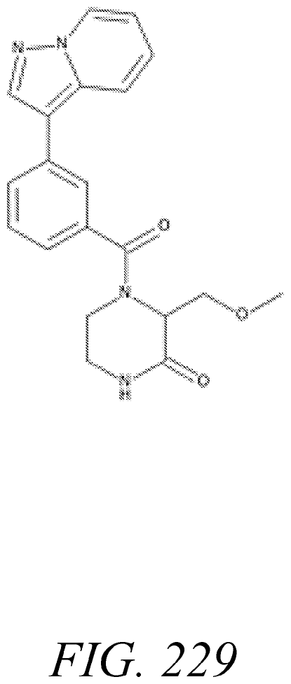

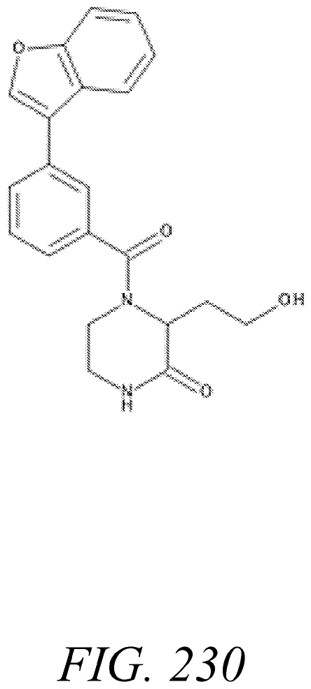

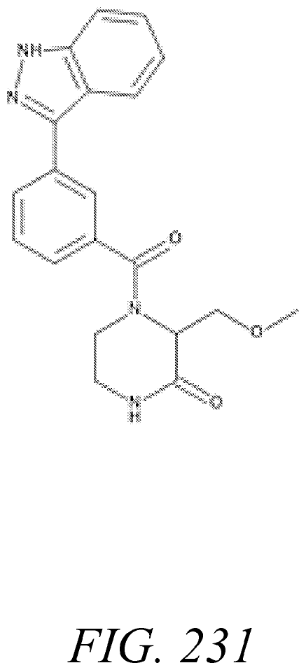

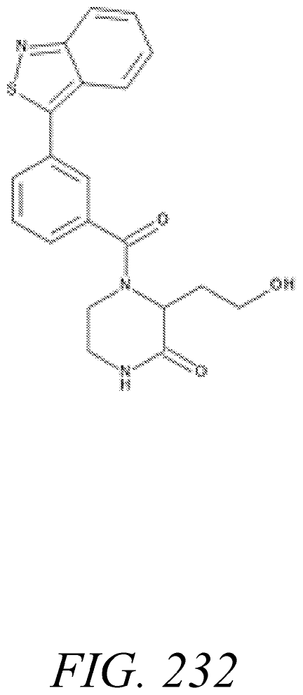

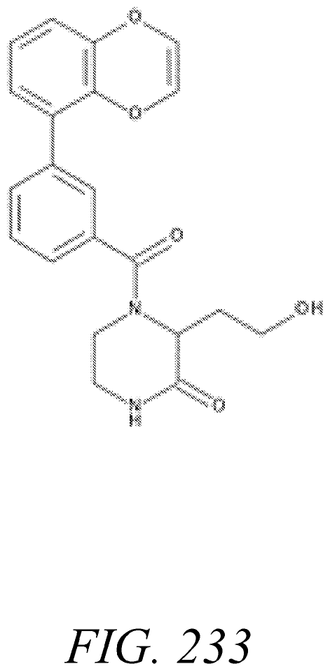

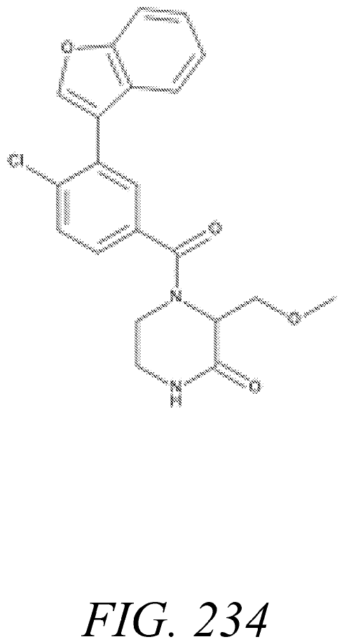

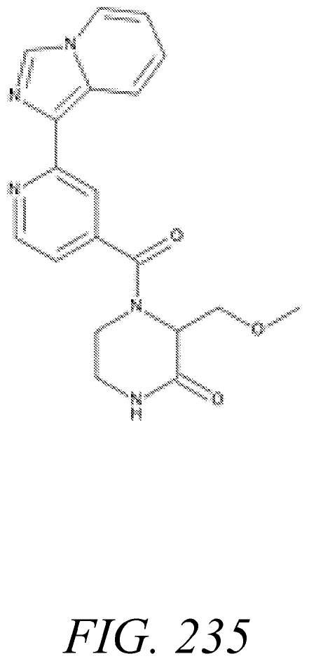

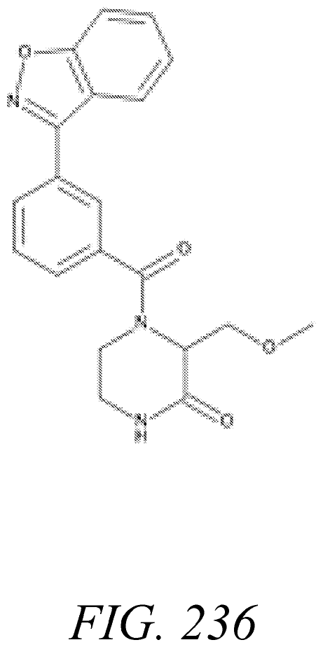

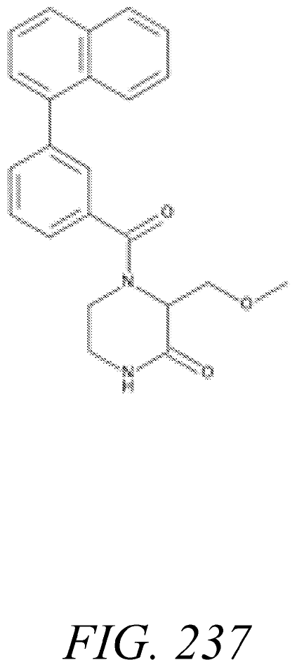

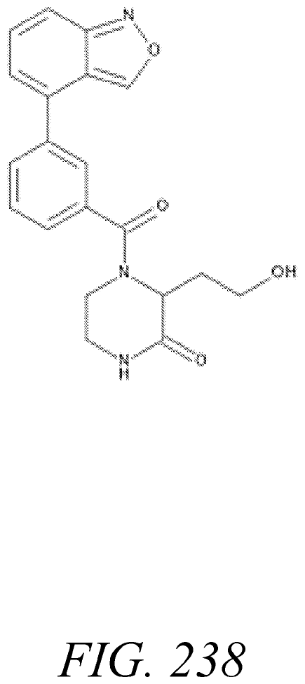

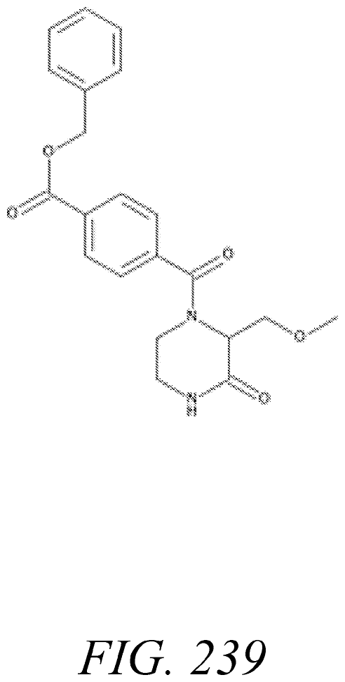

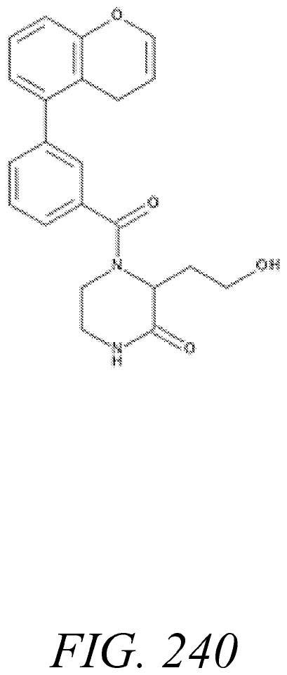

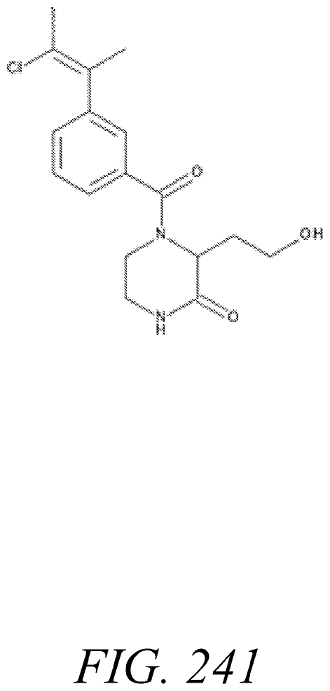

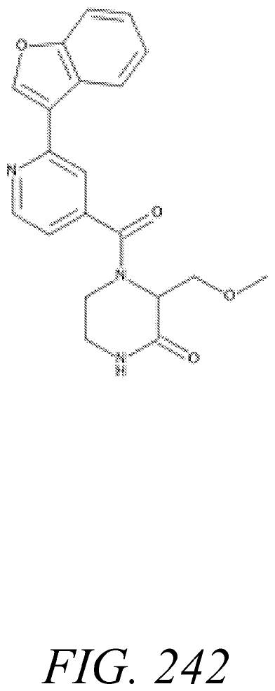

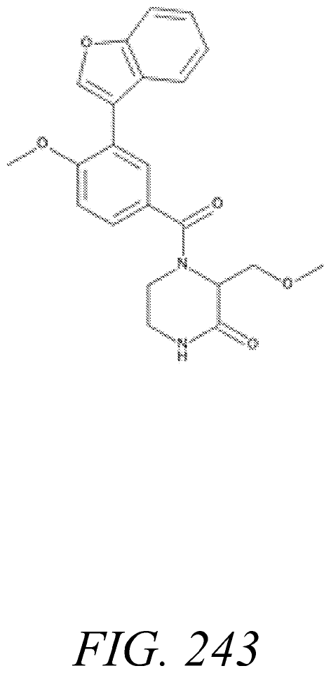

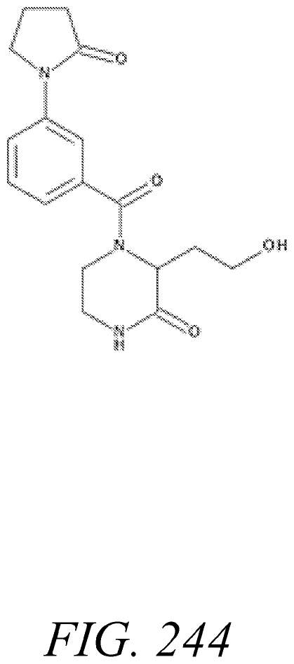

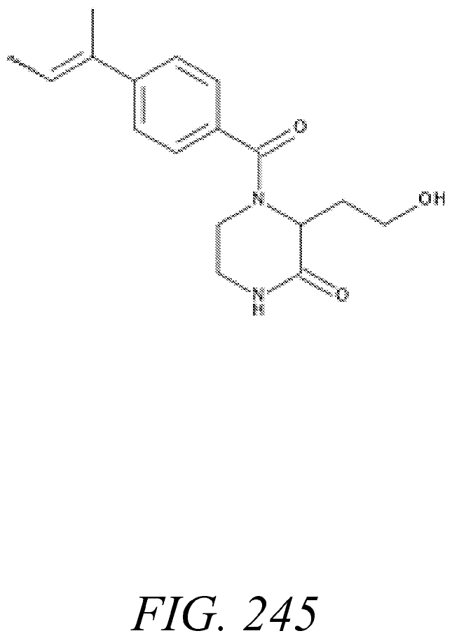

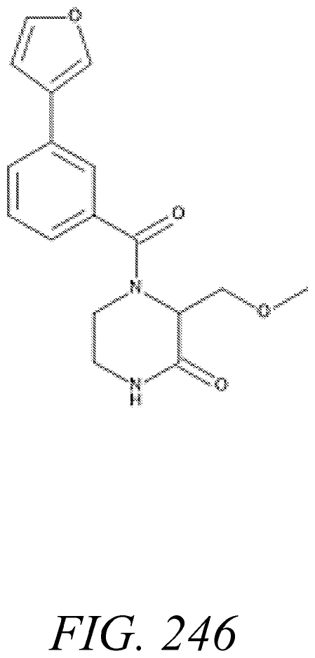

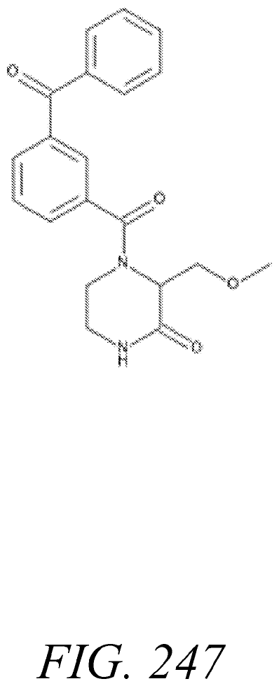

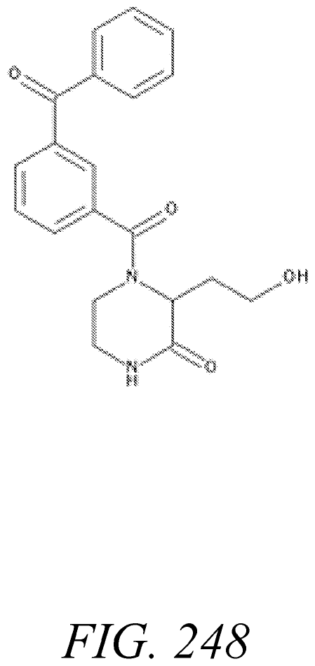

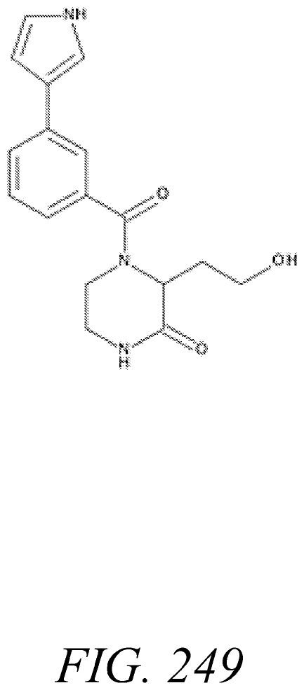

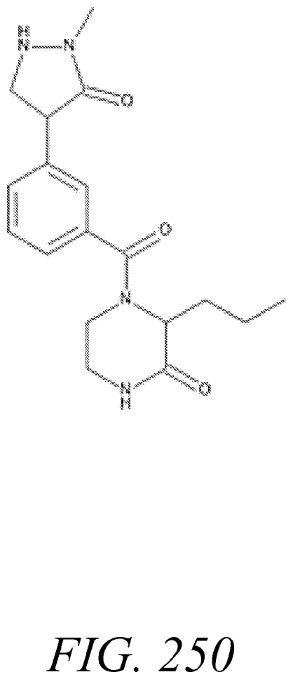

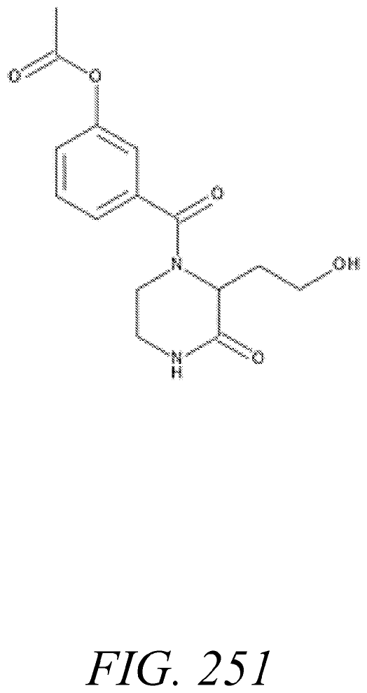

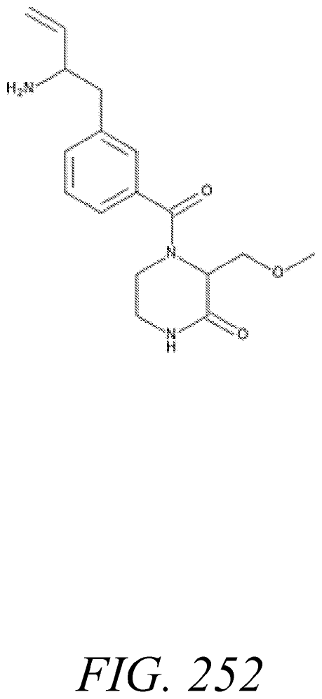

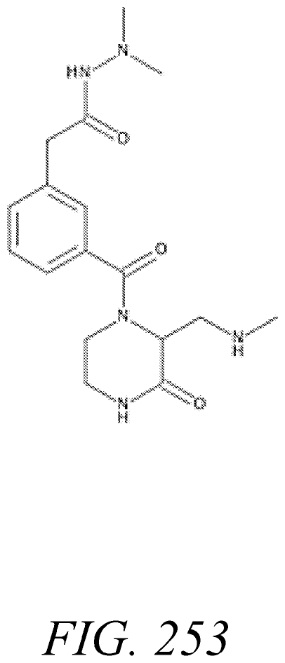

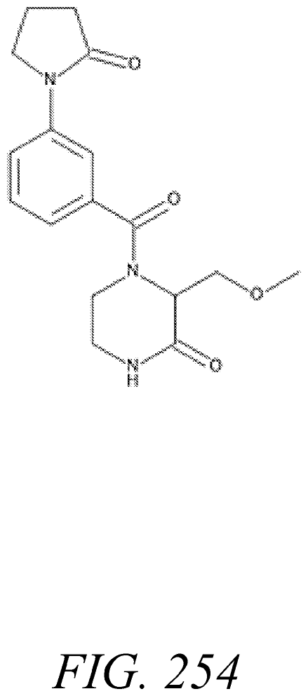

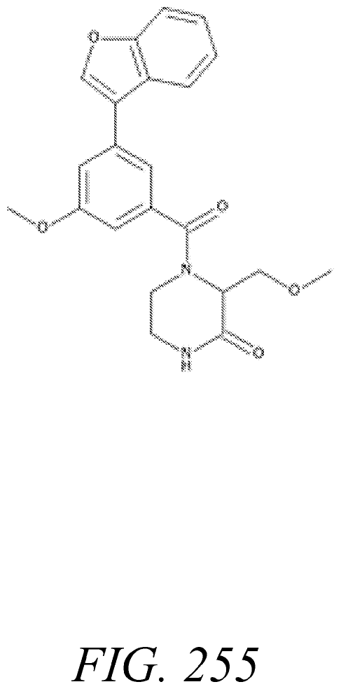

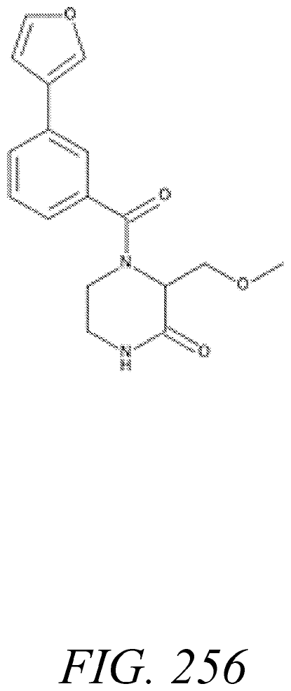

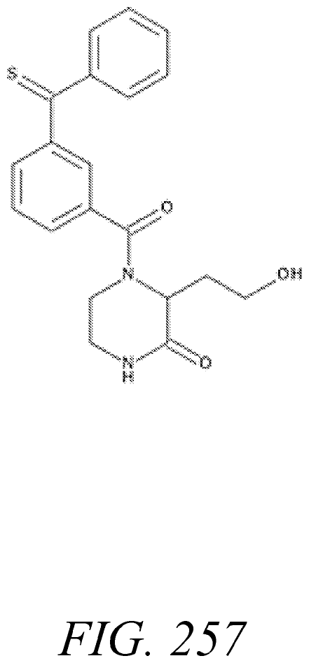

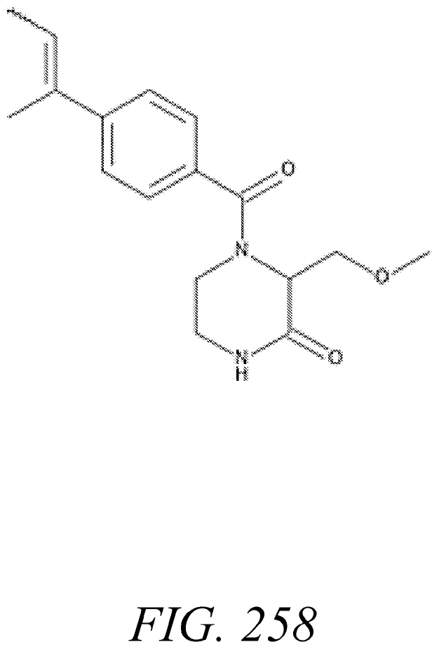

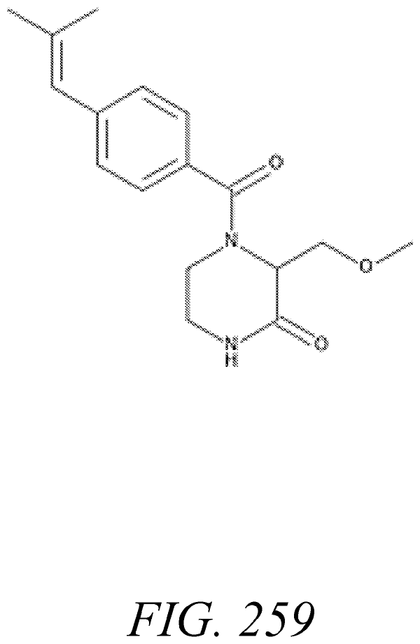

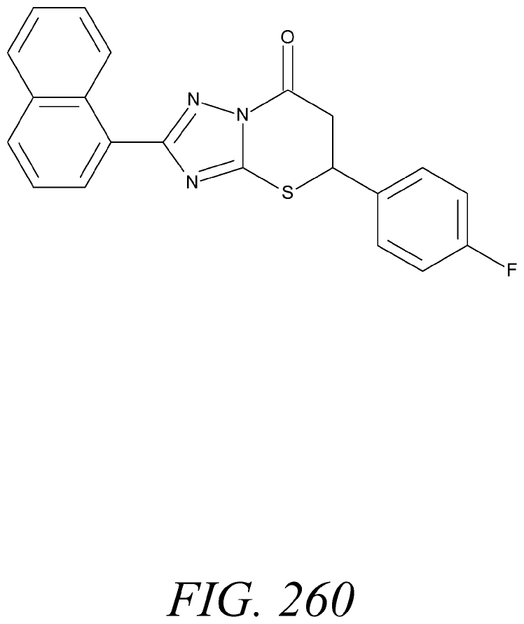

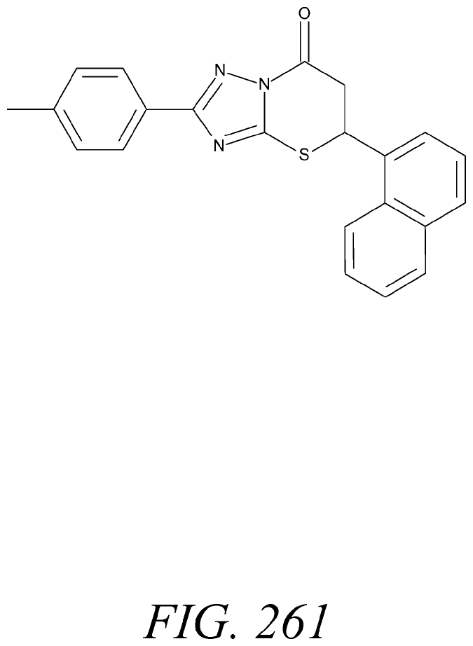

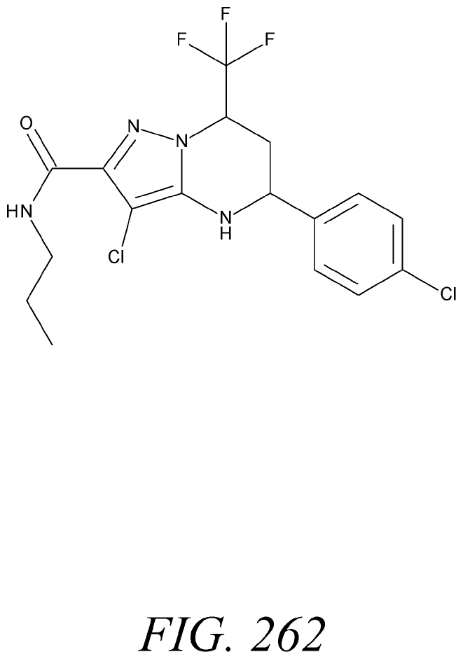

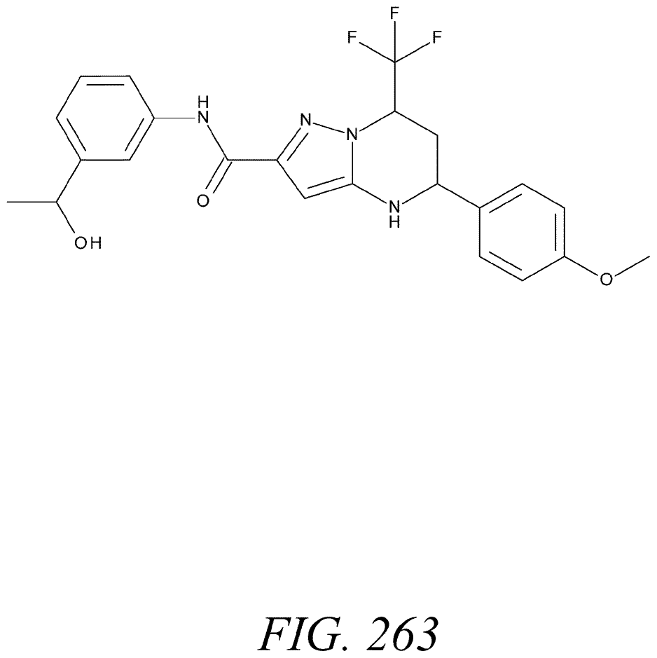

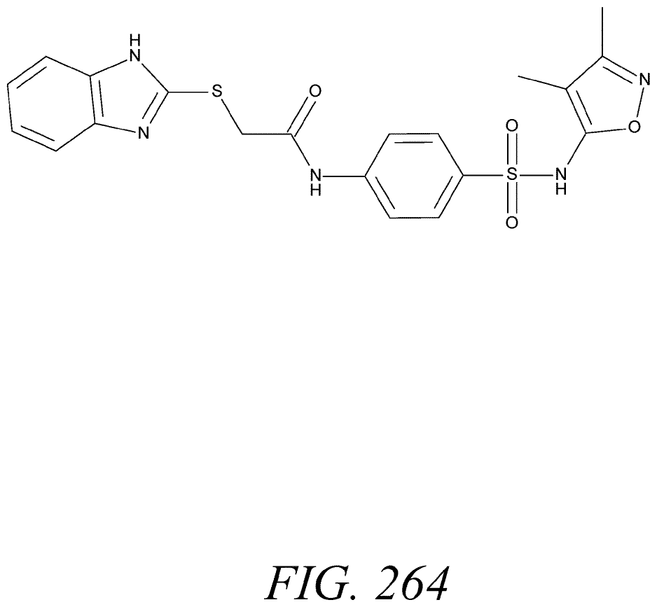

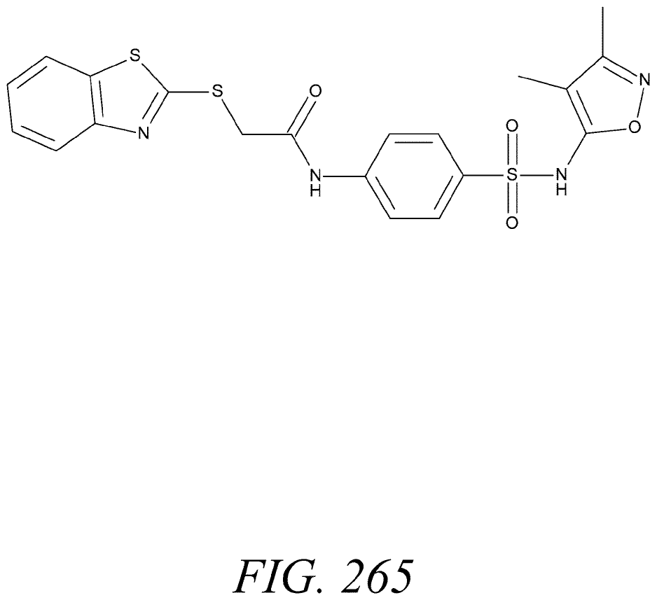

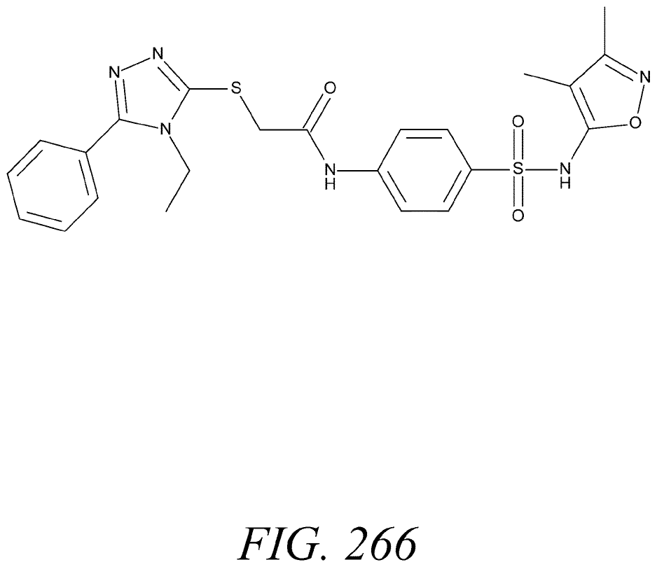

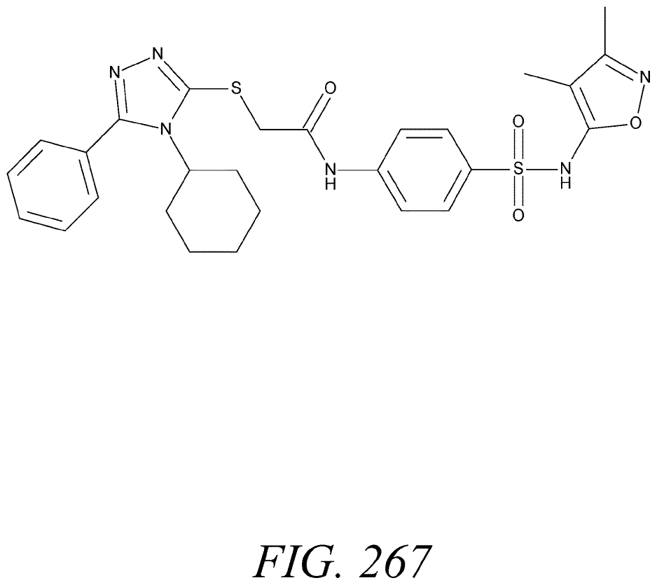

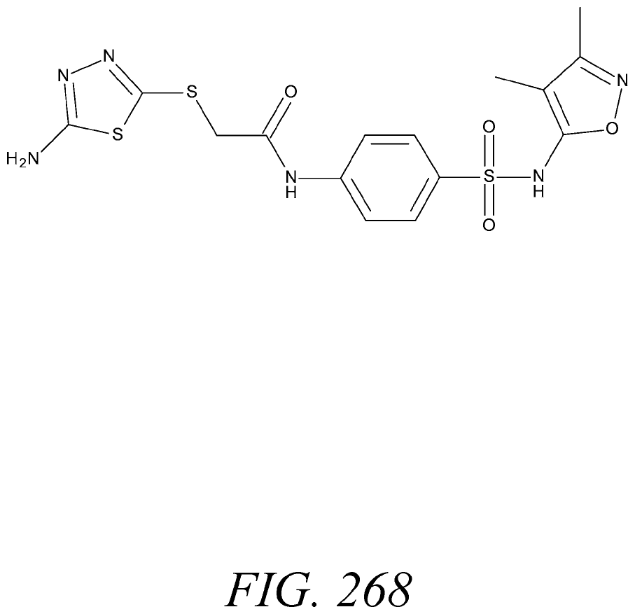

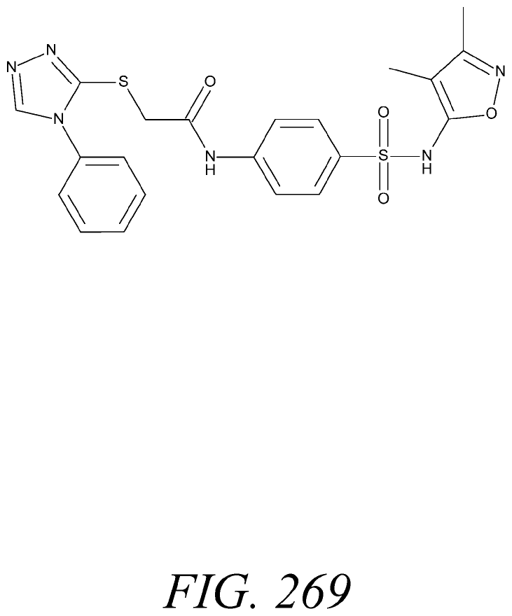

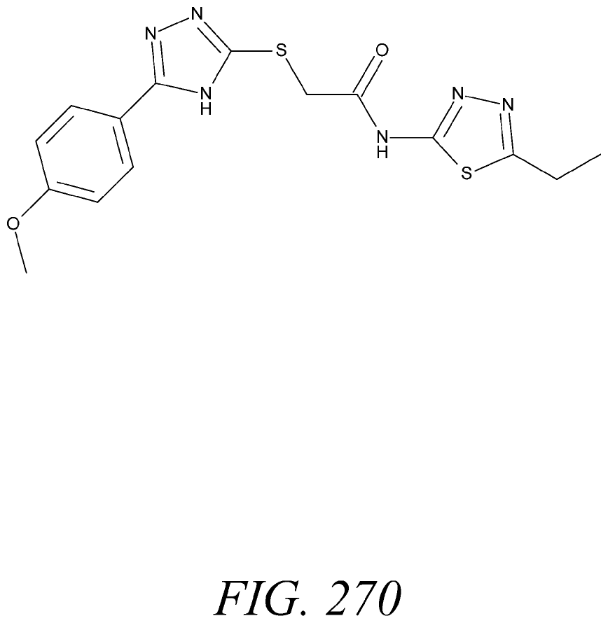

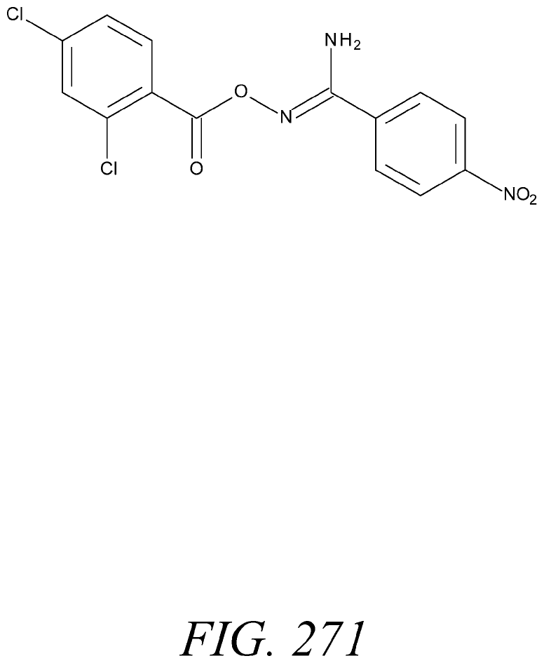

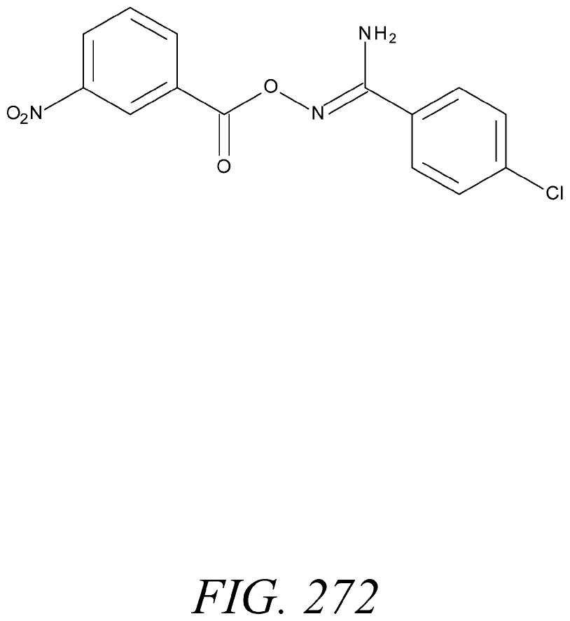

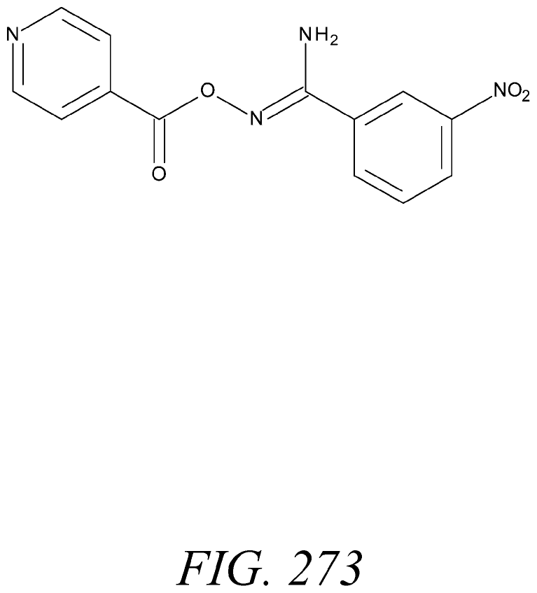

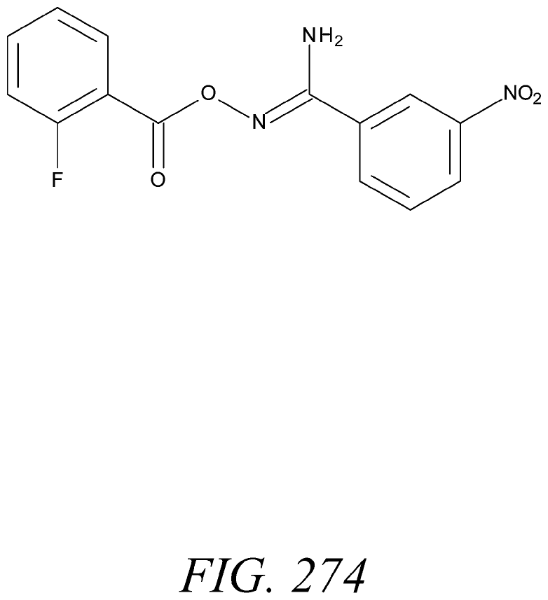

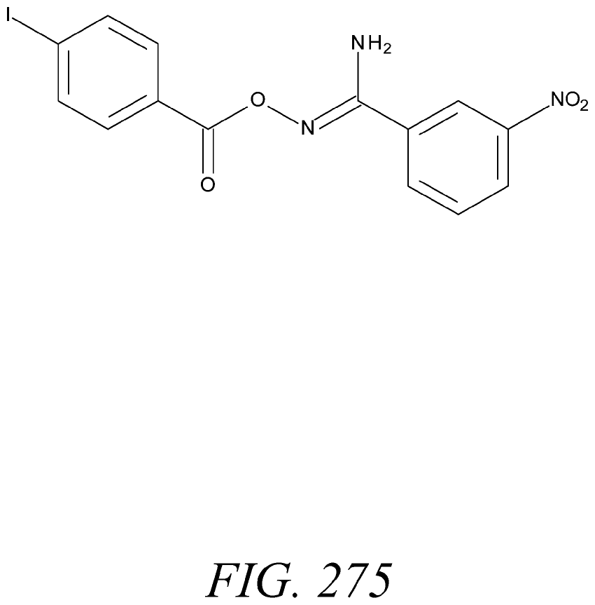

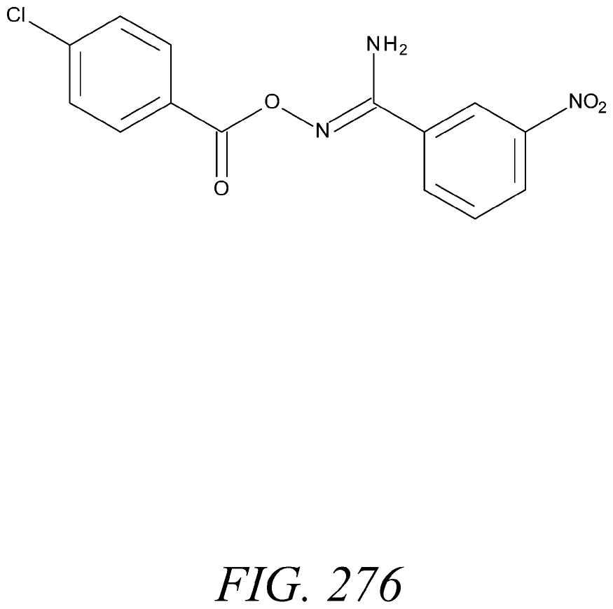

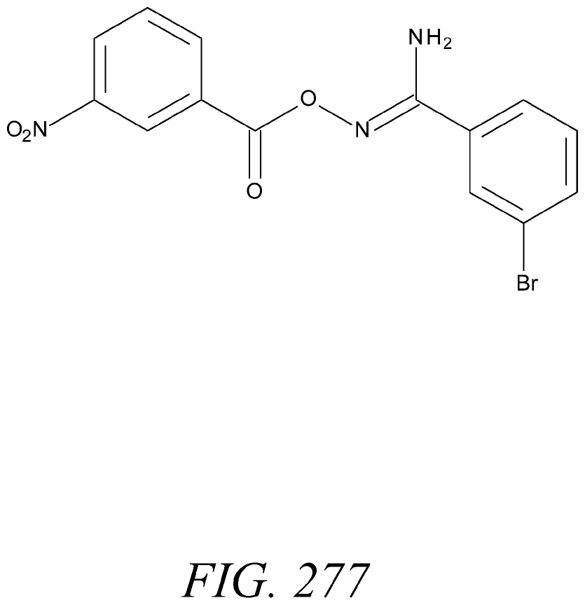

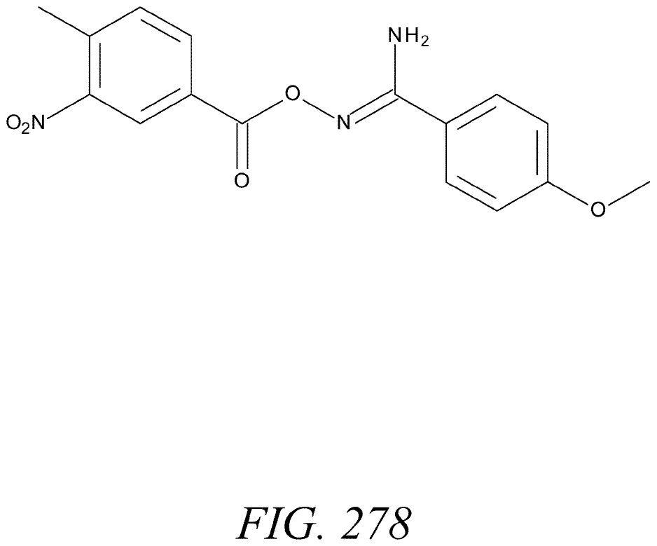

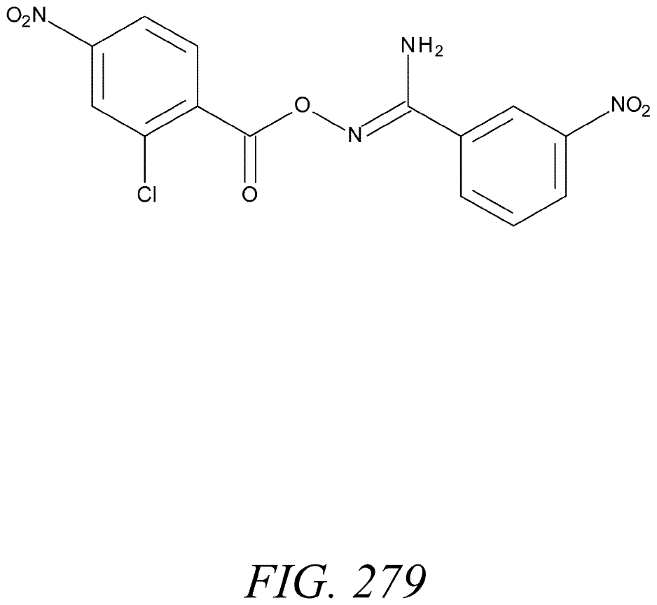

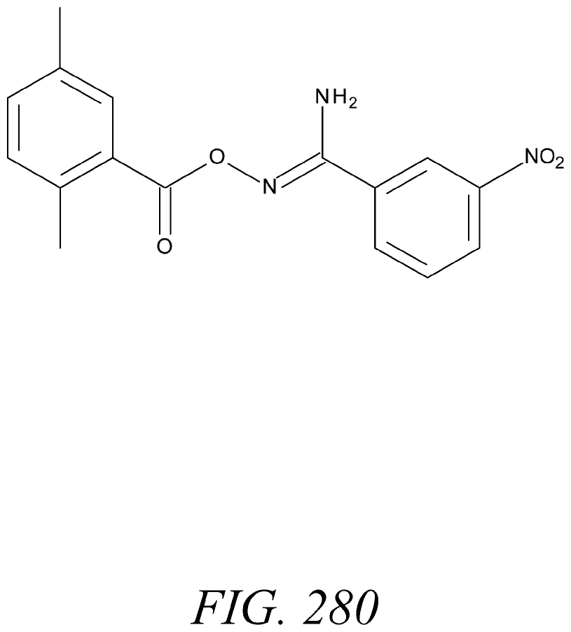

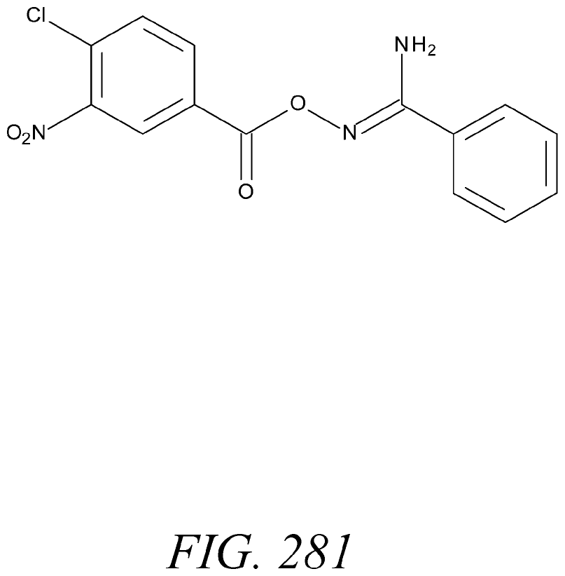

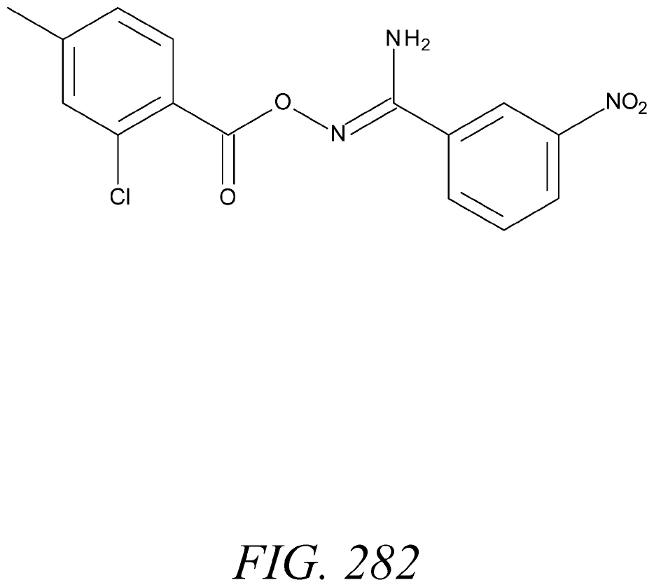

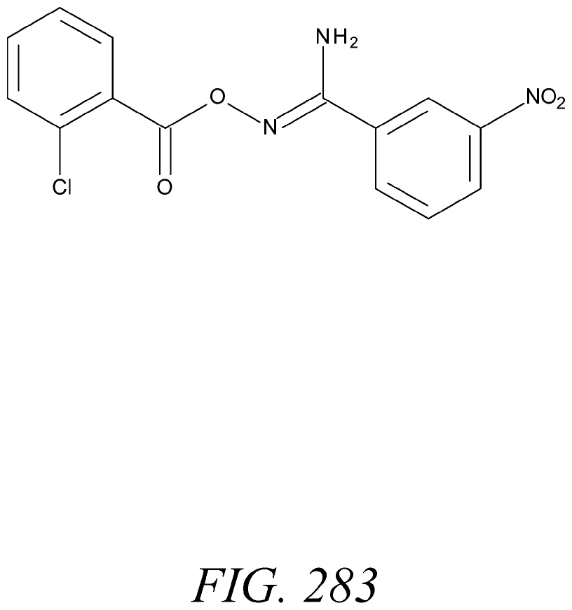

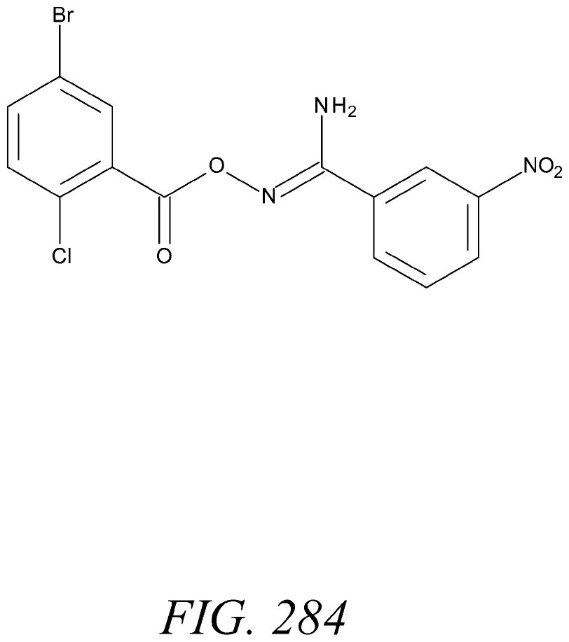

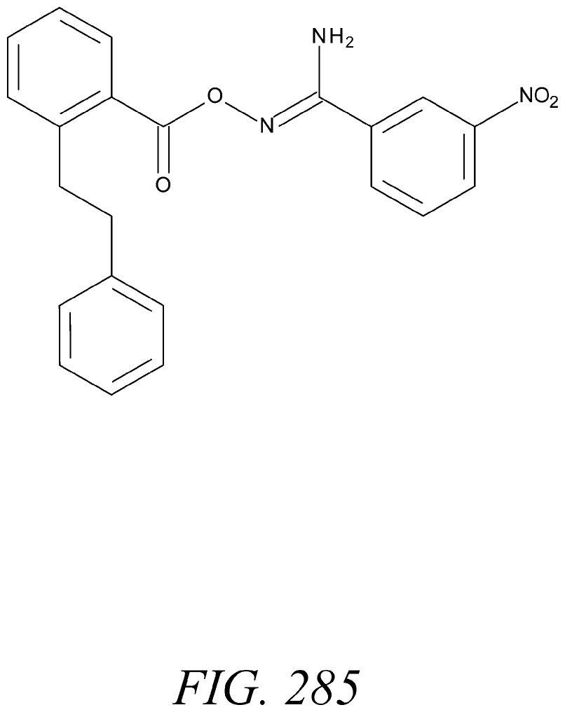

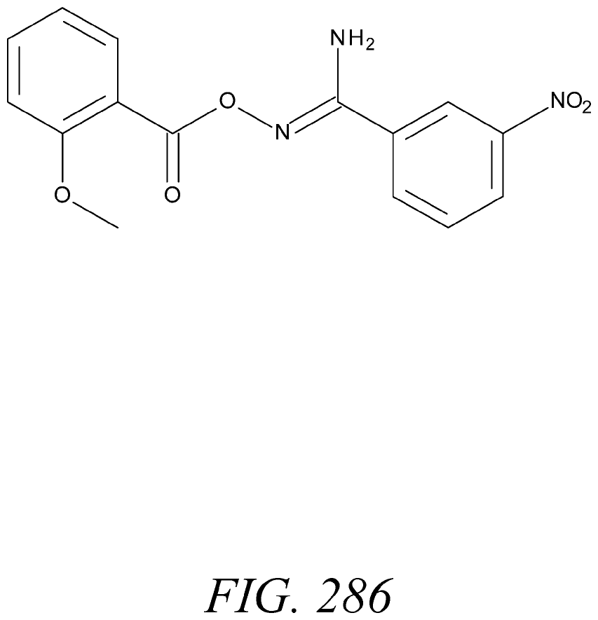

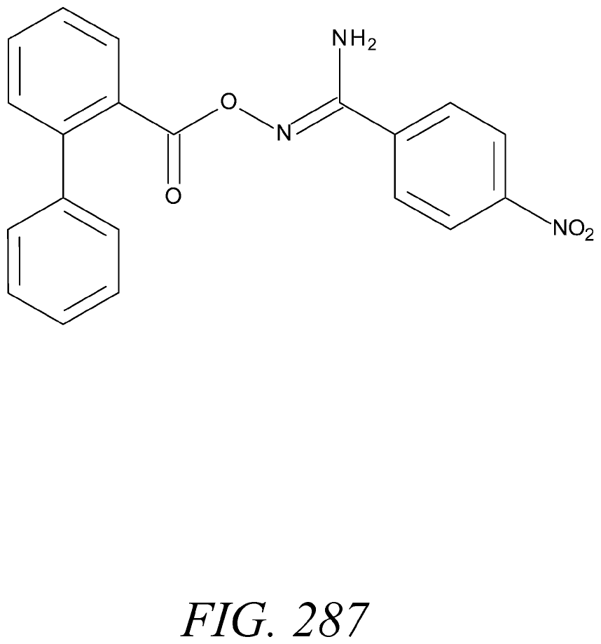

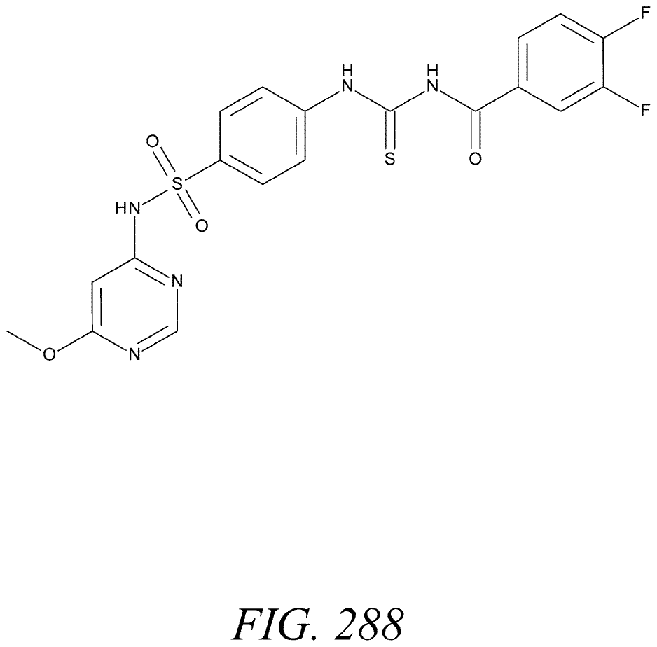

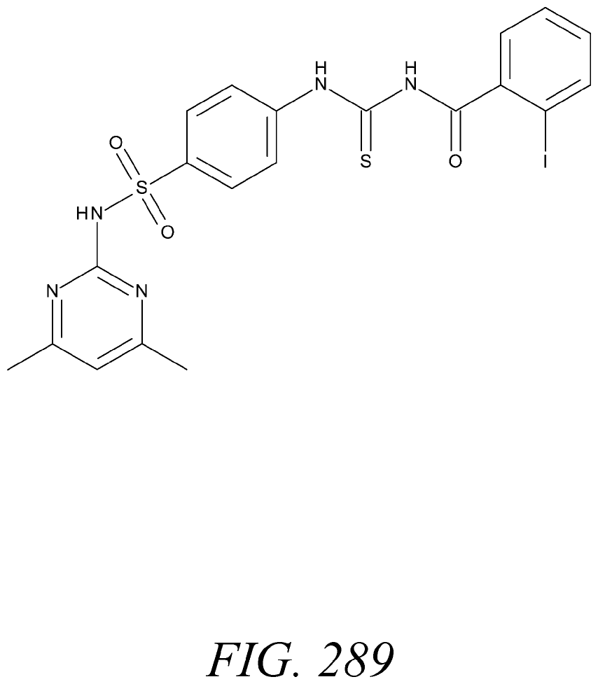

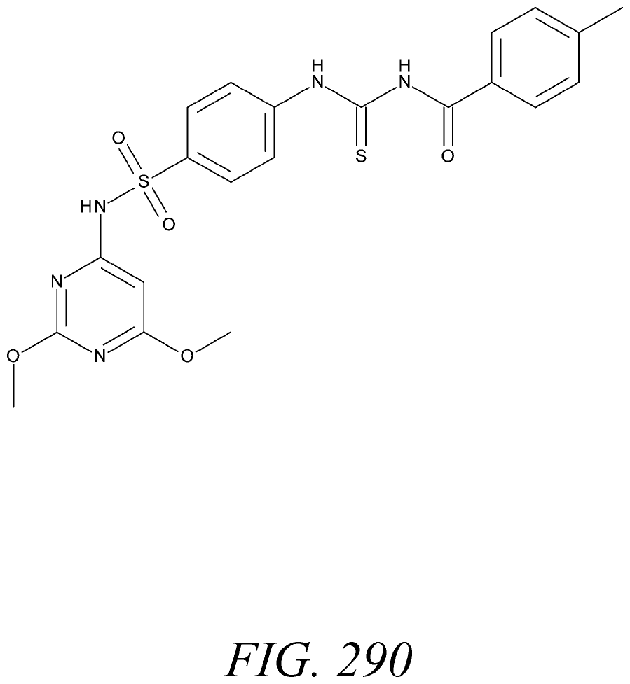

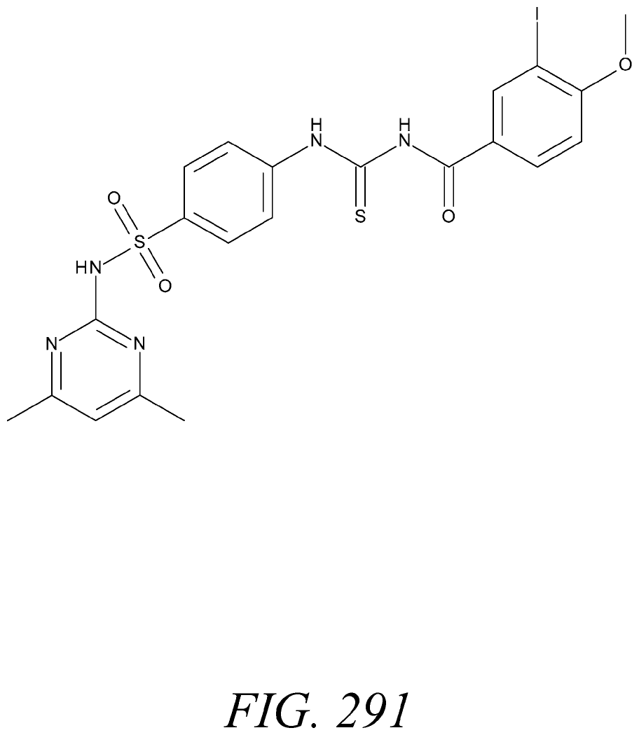

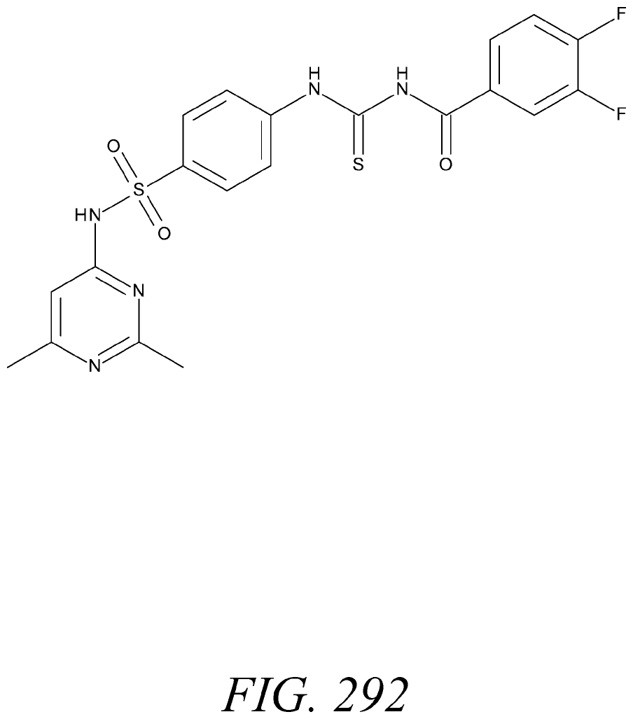

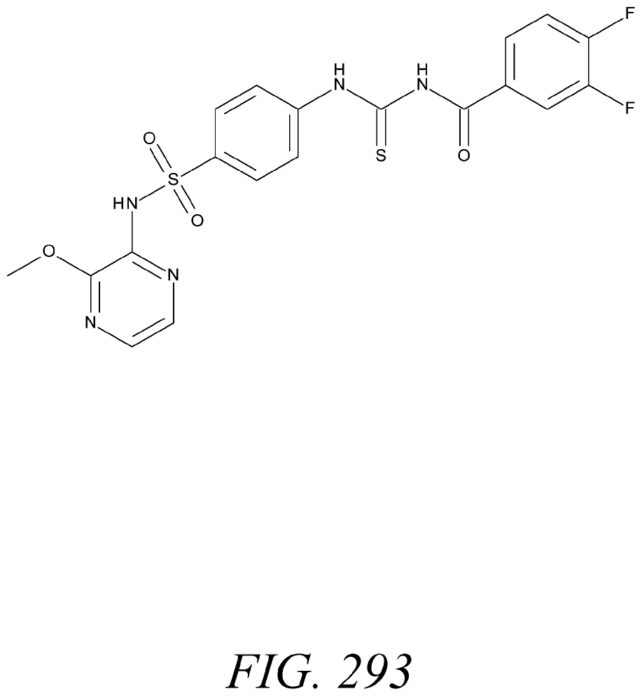

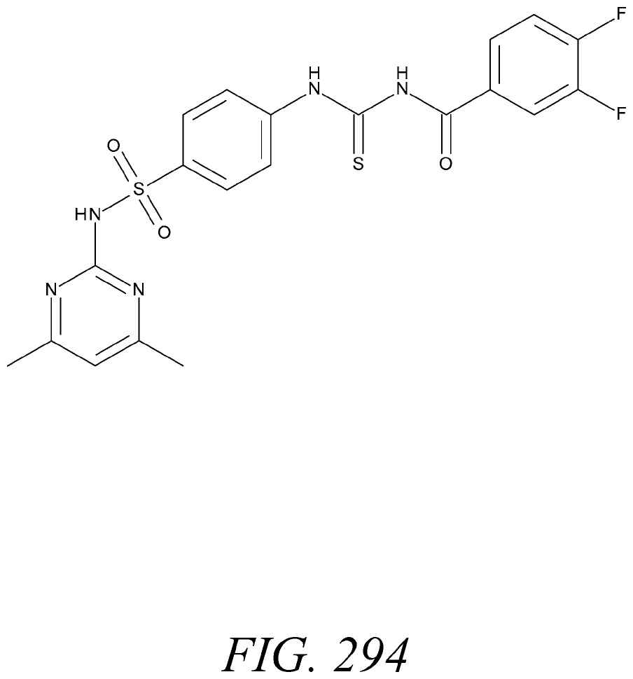

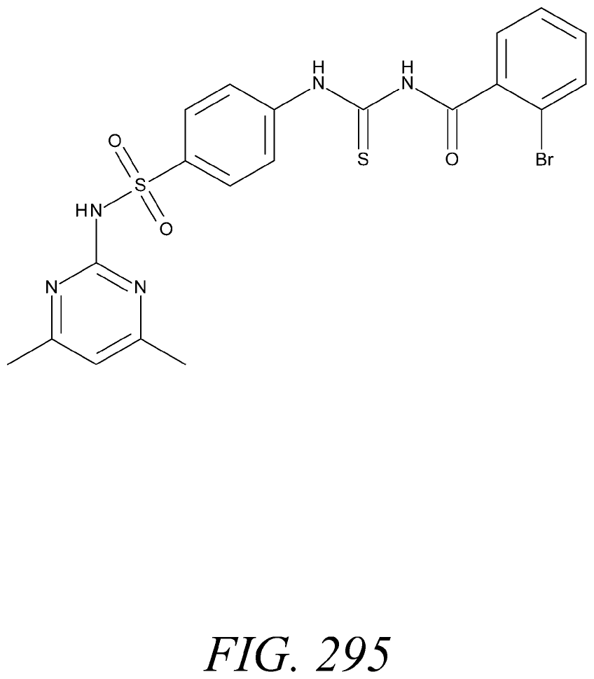

TABLE 33
Sirtuin modulating compounds identified by activity tests

| | | Number of | | Standard | |
|---|---|---|---|---|---|
| | Compound | replicates | Activity % | deviation | Conclusion |
| Formula | FIG. 260 | 3 | 93.6 | 5.5 | Slight Inhibitor |
| III | FIG. 261 | 6 | 82.41 | 6.41 | Inhibitor |
| | FIG. 262 | 3 | 94.3 | 4.17 | Slight Inhibitor |
| | FIG. 263 | 3 | 95.2 | 8.63 | Slight Inhibitor |
| Formula | FIG. 264 | 3 | 91.14 | 2.94 | Slight Inhibitor |
| V | FIG. 265 | 3 | 75.2 | 7.21 | Inhibitor |
| | FIG. 266 | 2 | 75.25 | 3.84 | Inhibitor |
| | FIG. 267 | 2 | 94.24 | 4.79 | Slight Inhibitor |
| | FIG. 268 | 2 | 78.31 | 4.31 | Inhibitor |
| | FIG. 269 | 3 | 91.37 | 7.71 | Slight Inhibitor |
| | FIG. 270 | 3 | 85.4 | 6.23 | Inhibitor |
| Formula | FIG. 271 | 3 | 76.3 | 3.21 | Inhibitor |
| VII | FIG. 272 | 2 | 92.4 | 2.4 | Slight inhibitor |
| | FIG. 273 | 3 | 83.9 | 5.17 | Slight inhibitor |
| | FIG. 274 | 3 | 75.4 | 2.92 | Inhibitor |
| | FIG. 275 | 3 | 80.1 | 3.84 | Inhibitor |
| | FIG. 276 | 3 | 71.5 | 5.64 | Inhibitor |
| | FIG. 277 | 3 | 81.6 | 3.9 | Inhibitor |
| | FIG. 278 | 3 | 72.8 | 5.82 | Inhibitor |
| | FIG. 279 | 3 | 91.5 | 4.72 | Slight Inhibitor |
| | FIG. 280 | 3 | 80.9 | 6.16 | Inhibitor |
| | FIG. 281 | 3 | 66.0 | 2.22 | Strong Inhibitor |
| | FIG. 282 | 3 | 71.9 | 4.9 | Inhibitor |
| | FIG. 283 | 3 | 84.5 | 3.4 | Inhibitor |
| | FIG. 284 | 3 | 77.1 | 4.55 | Inhibitor |
| | FIG. 285 | 3 | 70.3 | 2.47 | Inhibitor |
| | FIG. 286 | 3 | 85.8 | 2.75 | Inhibitor |
| | FIG. 287 | 3 | 77.4 | 7.21 | Inhibitor |
| Formula | FIG. 288 | 2 | 89.4 | 0.42 | Inhibitor |
| XI | FIG. 289 | 3 | 70.4 | 0.39 | Inhibitor |
| | FIG. 290 | 3 | 92.4 | 1.3 | Slight inhibitor |
| | FIG. 291 | 3 | 60.3 | 7.29 | Strong Inhibitor |
| | FIG. 292 | 3 | 92.9 | 8.52 | Slight inhibitor |
| | FIG. 293 | 3 | 69.9 | 6.78 | Strong Inhibitor |
| | FIG. 294 | 3 | 80.9 | 4.53 | Inhibitor |
| | FIG. 295 | 3 | 53.4 | 8.79 | Strong Inhibitor |

### Steady State SIRT3 Activating Compounds

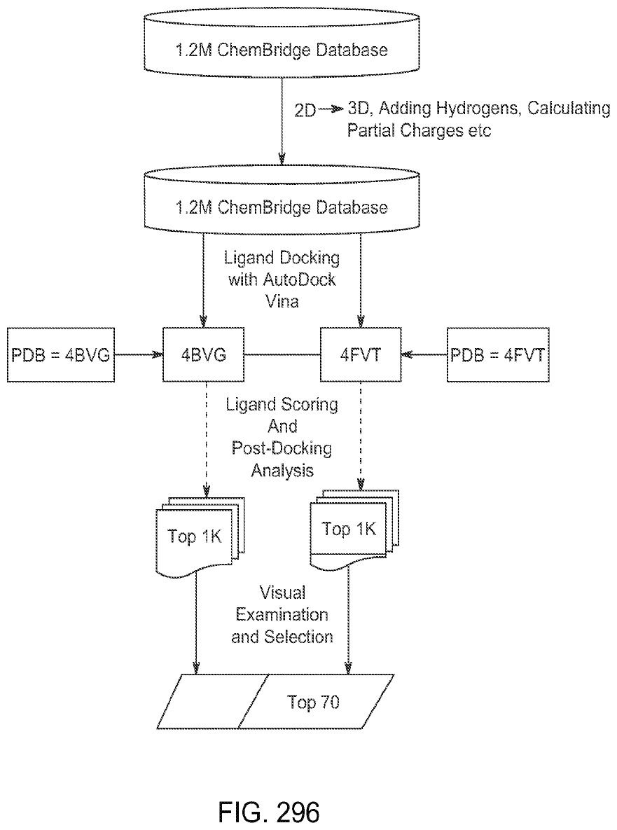

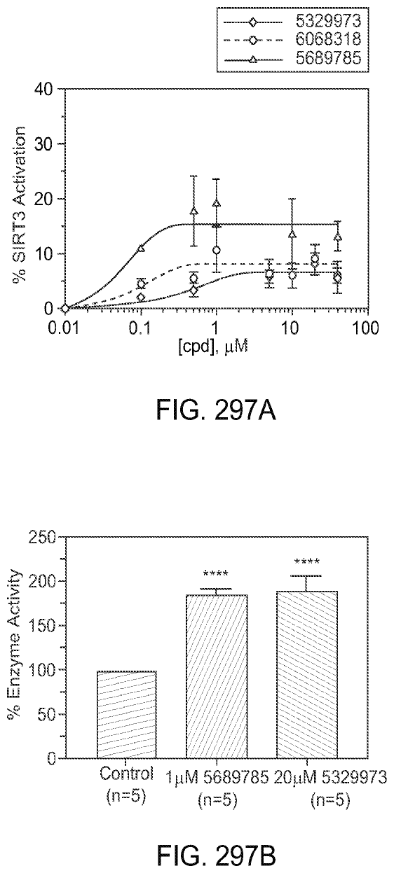

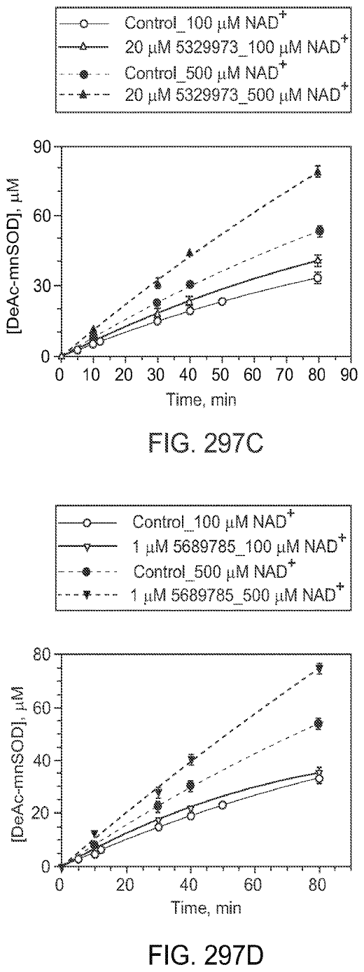

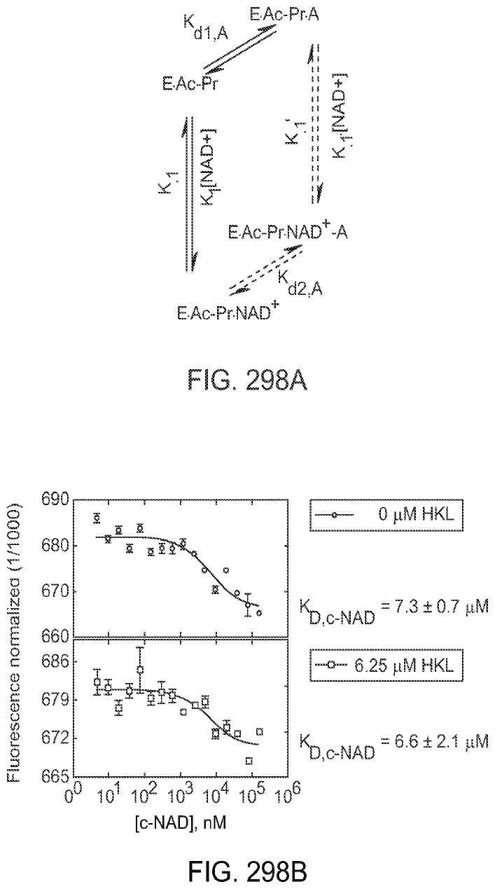

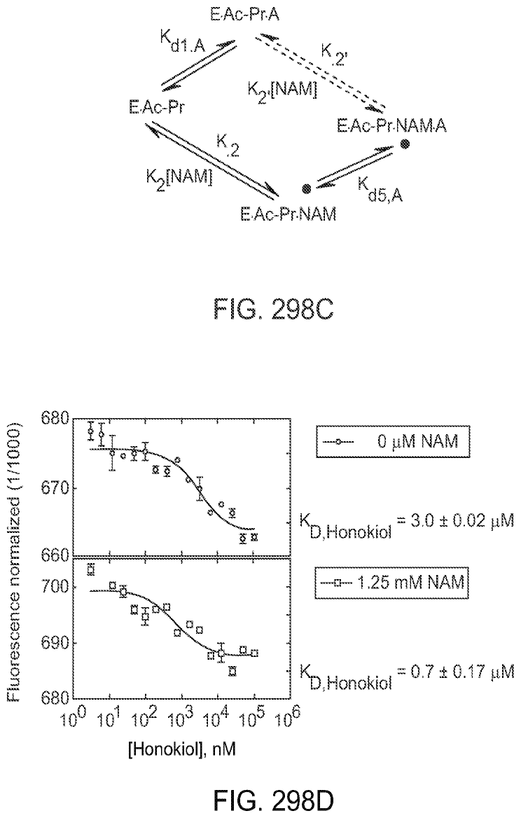

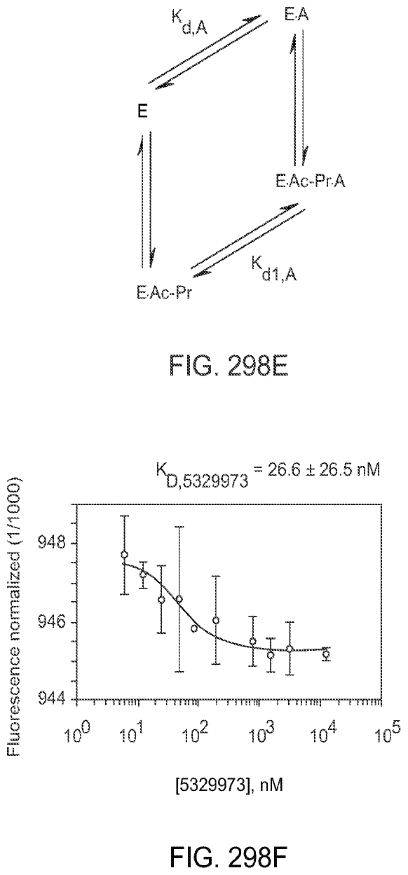

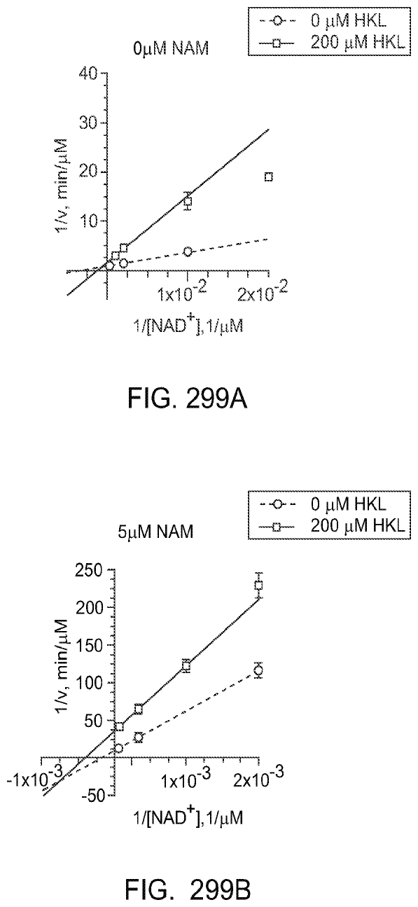

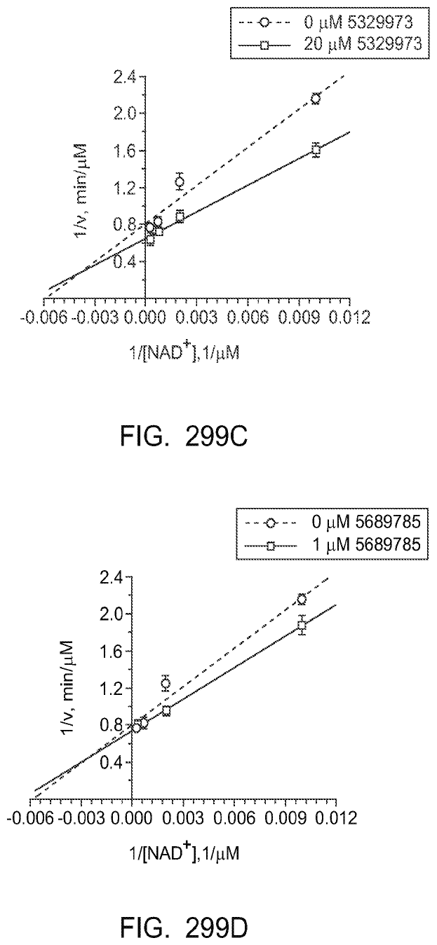

Importantly, our drug discovery workflow (FIG. 296) identified the first reported steady state activators of SIRT2 (Table 5), which improve qualitatively upon the properties of Honokiol and are ideal candidates for SIRT activator drug development. The steady state activators 5329973 (Formula VII) and 5689785 (Formula XI) were characterized and found to provide a 184.5% (at 20 μM) and 194.4% (at 1 μM) increase in SIRT3 activity, respectively, under steady state conditions at the physiologically relevant [NAD⁺]=100 μM (FIG. 297), with the catalytic efficiency (k_{cat}/Kₘ) of SIRT3 being nearly doubled by both of these compounds (Table 34). The MST binding affinity result for compound 5329973 falls in the nM range which indicates that compound 5329973 is a strong binder to SIRT3 (FIG. 298F). The steady state parameter estimation (Table 34) shows that the catalytic efficiency is improved primarily through reduction in Kₘ, as originally proposed in our prior theoretical study, which predicted that this is possible to achieve through modulation of the conformation of the flexible loop induced by active site binding of properly designed small molecules. The observed results in Table 34 and FIG. 299 (Michaelis-Menten plots) closely match the predicted properties for steady state mechanism-based sirtuin activators. Dose-response curves for compounds 5689785 and 6068318 (Formula III) (FIG. 397A) also show that these compounds achieve over 50% of their maximal activation effect (AC50) at 100 nM and <1 uM concentrations respectively, which is promising for further development of these hits into drug-like leads.

TABLE 34
Model parameter estimates from global nonlinear fitting
of Michaelis-Menten for SIRT3 in the presence and absence of
1 μM 5689785 and 20 μM 5329973. [E₀] = 1.568 μM.

| | Control | 20 μM 5329973 | 1 μM 5689785 |
|---|---|---|---|
| Vₘₐₓ, μM/min | 1.381 ± 0.114 | 1.58 ± 0.0617 | 1.381 ± 0.029 |
| Kₘ, μM | 270.8 ± 94.8 | 172.8 ± 32.7 | 142.6 ± 16.3 |
| Kₘ/Vₘₐₓ, min | 211.4 ± 56.6 | 109.4 ± 16.4 | 108.2 ± 9.9 |

Goodness of Fit

| | | | |
|---|---|---|---|
| Degrees of Freedom | 2 | 2 | 2 |
| R square | 0.9539 | 0.9827 | 0.9932 |
| Absolute Sum of | 0.01999 | 0.008505 | 0.002213 |
| Squares | | | |
| Sy.x | 0.09997 | 0.06521 | 0.03326 |

SIRT3 activation by these steady state activators was demonstrated under both saturating NAD⁺ (FIG. 297A) and saturating peptide substrate (FIG. 297B-D) conditions. By approximately doubling the deacetylation rate of SIRT3 at 100 μM NAD⁺, which is similar to the NAD concentration in aged mitochondria since mitochondrial NAD⁺ is ˜200 μM in young age and can drop by 50% or more in old age, the level of SIRT3 activation by these compounds is sufficient to largely recover the mitochondrial deacetylation activity of SIRT3 characteristic of young age. Moreover, steady state activators like these compounds will be less affected by variations in physiological conditions pertinent to SIRT3 activity, such as enzyme expression level, than Honokiol.

### Materials and Methods

Activity assays—Activity Tests of Reference Compounds Table 5

### Chemicals and Reagents

MnSOD (KGELLEAI-(KAc)-RDFGSFDKF) was synthesized by GenScript (Piscataway, NJ). Human SIRT3 (recombinant) (His-tag), NAD⁺ (SIRT Substrate), FdL2 (QPKKAC-AMC) peptide, also called p53-AMC peptide, Nicotinamide, and HDAC buffer were purchased from Enzo Life Sciences (Farmingdale, NY). Dimethyl sulfoxide and trifluoroacetic acid were purchased from Sigma-Aldrich (St. Louis, MO). Tested compounds were purchased from ChemBridge Corporation (San Diego, CA). Carba-NAD was synthesized by Dalton Pharma (Toronto, ON).

Effect of Hit Compounds on hSIRT3¹⁰²⁻³⁹⁹ Deacetylation Activity—HPLC Assay Using Native Peptide

Enzymatic reactions included either 1000 μM NAD⁺ and 50 μM MnSOD peptide (Conditions I) or 50 μM NAD⁺ and 600 μM peptide substrate (Conditions II) in presence of hit compounds (50 μM (Conditions I) or 10 μM (Conditions II)), in a HDAC buffer (50 mM TRIS-HCl, pH 8.0, containing 137 mM sodium chloride, 2.7 mM potassium chloride, and 1 mM magnesium chloride) dimethyl sulfoxide solution 95/5. The reaction was initiated by adding 5 U or 50 U hSIRT3 102-399 and incubated at 37° C. for 30 min. The reaction was terminated by adding a stopping solution (final concentration of 2% TFA, 5 mM Nicotinamide). The peptide product and substrate were resolved using HPLC (reversed-phase C18 HPLC Column). Solvent A was composed of 90% HPLC grade water and 10% acetonitrile with 0.05% (v/v) TFA. Solvent B was composed of acetonitrile with 0.02% (v/v) TFA. A linear gradient was performed for 20 min from 0% B to 51% (v/v) B. Solvent B percentage was increased to 100% within 5 minutes. Then returned to the starting conditions (0% B) within 5 min. Solvent A percentage (100%) was maintained at 100% for 5 additional minutes (36 min total run time).

Effect of Hit Compounds on hSIRT3¹⁰²⁻³⁹⁹ Deacetylation Activity—Fluorescence-Based Assay Using Fluorolabeled Peptide

The modulation effect of hits compounds for SIRT3¹⁰²⁻³⁹⁹ deacetylation activity was determined using FdL2 peptide. The enzymatic reactions were carried out similar to as described above. The reaction was terminated by adding the 1× developer 2 mM NAM solution and measured the fluorescence on TECAN microplate reader. The raw data were fitted to the defined model equations using GraphPad Prism (GraphPad Software, Inc, CA).

Activity assays—Activity tests Table 33

### Chemicals and Reagents

MnSOD (KGELLEAI-(KAc)-RDFGSFDKF) was synthesized by GenScript (Piscataway, NJ). Human SIRT3 (recombinant) (His-tag), NAD⁺ (SIRT Substrate), Nicotinamide and HDAC buffer were purchased from Enzo Life Sciences (Farmingdale, NY). Dimethyl sulfoxide and trifluoroacetic acid were purchased from Sigma-Aldrich (St. Louis, MO). Tested compounds were purchased from ChemBridge Corporation (San Diego, CA).

### HPLC Assays

Enzymatic reactions included either 1000 μM NAD⁺ and 50 μM MnSOD peptide in presence of hit compounds (50 μM) (Conditions I), in a HDAC buffer (50 mM TRIS-HCl, pH 8.0, containing 137 mM sodium chloride, 2.7 mM potassium chloride, and 1 mM magnesium chloride) dimethyl sulfoxide solution 95/5. The reaction was initiated by adding 5 U hSIRT3 102-399 and incubated at 37° C. for 30 min. The reaction was terminated by adding a stopping solution (12% TFA, 30 mM Nicotinamide). The peptide product and substrate were resolved using HPLC (reversed-phase C18 HPLC Column). Solvent A was composed of 90% HPLC grade water and 10% acetonitrile with 0.05% (v/v) TFA. Solvent B was composed of acetonitrile with 0.02% (v/v) TFA. A linear gradient was performed for 20 min from 0% B to 51% (v/v) B. Solvent B percentage was increased to 100% within 5 min. Then returned to the starting conditions (0% B) within 10 min. Solvent A percentage (100%) was maintained at 100% for 10 additional minutes (41 min total run time).

### Virtual Screening for Million Compounds (Tables 3 and 4)

### High Throughput Batch Docking

A database containing 1.2 million compounds (Chembridge) was screened against two SIRT3 structures (PDB: 4FVT and 4BVG). Our prior knowledge of the bioactive conformations of HKL and its binding sites of the SIRT3 were applied to define the docking site and the selection of the protein conformation. The structure-based docking was used in a sequential process to implement VS (FIG. 299). In brief, these 1.2 million compounds were first imported into a database and converted from 2D→3D using relevant modules in MOE. Next, other modules (iMOE) were used to add hydrogens as required, calculate partial charges and perform a quick round of energy minimization using the MMFF94x forcefield. When appropriate, the most likely tautomeric form was generated and automatically chosen.

FIG. 299. is a schematic illustrating the compound screening process employed in the present application. A database containing 1.2 μM compounds was subjected to a series of operations which started with conversion from 2D to 3D and ended with a quick energy minimization for each compound. These compounds were then, independently, docked with two SIRT3 receptor structures (4BVG and 4FVT). The best poses for top 1,000 compounds (by calculated binding energy) in each docking run were chosen for further post-docking analysis-which was performed using MOE as described in post-docking analysis. The final step involved careful examination and selection of 20 compounds, from both docking runs, which gave us the best coverage of scaffold diversity, lack of repetition and maximum number of favorable interactions with residues at putative binding site.

These prepared ligand structures were converted back into the input file format preferred by AutoDock Vina. Our prior studies of the bioactive conformations of HKL and its binding sites in SIRT3 were applied to define the docking site and the selection of the protein conformation. Dockings were performed using AutoDock Vina on AWS cloud server. The grid center and volume were defined as, for 4FVT, Center (x, y, z)=23.19, 31.19, −14.31; Volume=29.6×34.8×33.8. For 4BVG, Center (x, y, z)=22.24, 30.39, −7.98; Volume=25.0×25.0×25.0. 20 possible conformations for each ligand were collected.

### Post Docking Analysis

The docked ligands were then ranked by docking scores in AutoDock Vina (predicted affinity in kcal/mol). The top 1,000 compounds were then screened for (1) the formation of non-covalent interactions with 4FVT and 4BVG binding cavity; (2) formation of hydrogen bonds with residues lining binding site; (3) interaction within specific residues within the co-factor binding loop of SIRT3; (4) predicted ADME properties, such as Log P, Log D and (5) predictions about synthetic feasibility. Only the best ranked pose of each compound from top 1,000 were included in the list.

This list of top 1,000 compounds was then subjected to further post-docking analysis using MOE. The ligands were imported into a database and converted into the native format utilized by MOE after addition of hydrogen atoms and recalculation of partial charges for each structure. A small percentage of these structures were carefully examined to ascertain that this processing had not introduced any undesirable artifacts in them. Next a truncated post-docking energy minimization of ligands and hydrogen atoms from adjacent residues was performed to further optimize the interactions between docked poses and site residues, using a MOE script known as ‘analysis_dock.svl’. The energy optimization step provides a new value for approximate calculated Binding Energy of the compounds (in MOE). The optimized structures were then used for all subsequent post-docking analysis steps.

We screened for the presence of H_Bonds between optimized poses and residues lining the binding site. Based on previously docked structure of Honokiol, the following residues were considered to be part of the site: 4FVT (G145, S149, G153, G163, L168, Y171, F180, K195, E198, Y204, Q228 and H248). 4BVG (D156, P176, 1179, F294, S321, E323 (and NLE7 from the co-crystallized Ac-ACS peptide chain). The docked pose of Honokiol was also used to measure the relative ‘receptor buriedness’ of optimized poses in both receptor structures. The BHB (buriedness, hydrogen bonding, and binding energy) score for each compound was calculated using the general formula first described by Feher M. et al.

### Effect of Hit Compounds on h SIRT3¹¹⁸⁻³⁹⁹ Deacetylation Activity—Fluorescence-Based Assay Using Fluorolabeled Peptide

The modulation effect of hits compounds for SIRT3¹¹⁸⁻³⁹⁹ deacetylation activity was determined using FdL2 peptide. The enzymatic reactions were carried out similar to as described above. The reaction was terminated by adding the 1× developer 2 mM NAM solution and measured the fluorescence on TECAN microplate reader. The raw data were fitted to the defined model equations using GraphPad Prism (GraphPad Software, Inc, CA).

### Dockings Studies on Hit Compounds

### MD Simulation

Computational studies were performed using MOE (Molecular Operating Environment from Chemical Computing Group Inc., Montreal, PQ) version MOE 2020.09 on computers with quad-core Intel x86-64 (Intel Core i7-8th Generation) processors, 16 GB of RAM and running Windows 10 Pro. MMFF94x force field and solvation, as implemented in MOE, was used for all computational studies.

### Ligand Docking Procedure

Two methods were used to define the ligand-binding site in receptor protein structures. The first one, used for 4BVG, defined the site using the co-crystallized ligand (EX-527). The second, employed for 4FVT and 4BVG, utilized the ‘Site Finder module’ in MOE to create and place dummy atoms into potential ligand-binding sites for both structures. This step was performed after removing all molecules of minor compounds present within receptor structures such as sulphate ions, glycerol, 1,2-ethanediol etc. Fortuitously, the major site identified by second method for 4BVG was almost identical to the one defined by co-crystallized ligand. Hence it was assumed that the main putative (and internal) ligand-binding site identified in 4FVT was the best candidate for docking studies in that receptor structure.

Energy-optimized models of selected compounds were sequentially docked into these sites by ‘Dock’ application in MOE; default settings for parameters using the ‘Rigid Receptor’ protocol was employed for all docking studies. Top poses which exhibited reproducibility (RMSD<0.2 Å) in three independent docking simulations at a given site were the most optimal poses for that site. The main settings for ligand docking to were as follows: Docking Protocol=Rigid Receptor; Placement=Triangle Matcher; Rescoring=London dG; Retain=100, Refinement=Forcefield; Rescoring=GBVI/WSA dG; Retain=100 top poses. The default settings for docking parameters using ‘Rigid Receptor’ protocol was found to be satisfactory for docking of Honokiol and EX-527, which was used as test ligands. The top scoring pose for ligands from each docking run was used for calculating the approximate binding energy and other inputs for computation of the final docking scores.

### Calculation of Ligand Binding Energy

The binding energy for top docked poses of each ligand at each site was calculated using an updated version of a published method. It involved: calculating energies of the docked receptor-ligand complex (Ecpx); the unbound ligand in solution (Elig) and unbound solvated rigid receptor from the complex (Eprot) for use in the equation: Ebind=Ecpx−(Elig+Eprot). The top selected docked pose of each ligand, obtained from three independent docking runs, was used for calculation of the approximate ligand-binding energy. Optimization of semi-rigid (fixed heteroatoms) receptor-ligand complex was conducted under distance-dependent dielectric conditions, the final step being performed under solvation, using a regime under which only hydrogens in the ligand and cavity could undergo further structural optimization.

Binding energies calculated using molecular mechanics should always be viewed as approximations of the actual binding energy since any semi-rigid receptor approximation almost always underestimates effects of induced fit, whereas a fully flexible receptor approximation usually overestimates it. Hence, actual binding energy would be expected to lie between the rigid and flexible receptor values. The methodology used for the calculation of receptor buriedness for top docked (and energy optimized) ligand poses, as well as enumeration of their hydrogen bond interactions with residues in the binding site have been described previously; and the code is available upon request.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A sirtuin modulating compound of Formula VII and/or a salt thereof: 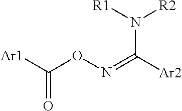
wherein R₁ and R₂ are independently selected from the group consisting of hydrogen, alkyl, heteroalkyl, cycloalkyl, and heterocycloalkyl; and wherein Ar1 and Ar2 are independently selected from aryl and heteroaryl, wherein the aryl and heteroaryl are optionally substituted with one or more substituents selected from the group consisting of (C₁-C₁₀)-alkyl, (C₁-C₁₀)-alkenyl, alkoxy, halo, amine, nitro, sulfonyl-alkyl, acyl, amide, acetylamine, alkyl amine, alkynyl, and hydroxyl.

2. The sirtuin modulating compound of claim 1, wherein R₁ and R₂ are independently selected from the group consisting of hydrogen and alkyl, and Ar2 is nitrobenzene.

3. The sirtuin modulating compound of claim 2, wherein Formula VII and/or a salt thereof is:

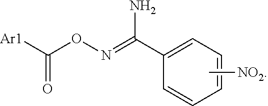

4. The sirtuin modulating compound of claim 1, wherein Formula VII and/or a salt thereof is: 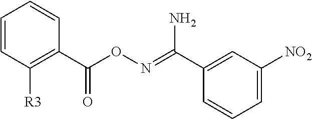
wherein R₃ is selected from the group consisting of hydrogen, alkyl, heteroalkyl, cycloalkyl, halo, and heterocycloalkyl.

5. The sirtuin modulating compound of claim 1, wherein the compound is a sirtuin activator.

6. The sirtuin modulating compound of claim 1, wherein the compound is a sirtuin inhibitor.

7. The sirtuin modulating compound of claim 1, wherein the compound modulates SIRT3 activity.

8. A sirtuin modulating compound of Formula III and/or a salt thereof: 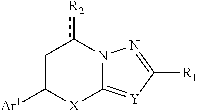
wherein Ar¹ is aryl or heteroaryl, R¹ is selected from the group consisting of hydrogen, alkyl, alkenyl, cycloalkyl, heterocycloalkyl, aryl, heteroaryl, amine, and —C(O)NR₃R₄, wherein the Ar¹ alkyl, alkenyl, alkynyl, acyl, alkoxy, alkenyloxy, cycloalkyloxy, cycloalkenyl, alkyl amine, acetamide, acetylamine, cycloalkyl, heterocycloalkyl, aryl, heteroaryl, and amine are optionally substituted with one or more substituents selected from the group consisting of (C₁-C₁₀)-alkyl, (C₁-C₁₀)-alkenyl, alkoxy, halo, amine, and hydroxy; and wherein R₃ and R₄ are independently selected from the group consisting of hydrogen, alkyl, alkenyl, aryl, and heteroaryl, and wherein R² is selected from the group consisting of hydrogen, alkyl, fluoroalkyl, alkenyl, aryl, heteroaryl, alkynyl, acyl, alkoxy, alkenyloxy, cycloalkyloxy, cycloalkenyl, amine, alkyl amine, and O, and wherein Y is selected from the group consisting of N and CH, and wherein X is selected from the group consisting of NH, S and O.

9. The sirtuin modulating compound of claim 8, wherein R₁ is selected from the group consisting of aryl and —C(O)NR₃R₄.

10. The sirtuin modulating compound of claim 8, wherein Formula III and/or a salt thereof is:

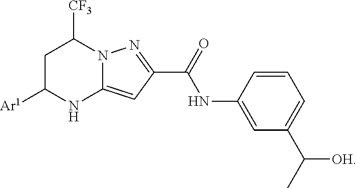

11. The sirtuin modulating compound of claim 8, wherein Formula TIT and/or a salt thereof is:

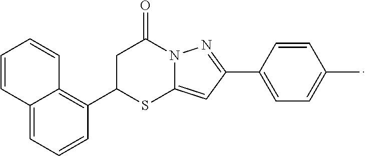 [SMILES: Cc1ccc(-c2cc3n(n2)C(=O)CC(c2cccc4ccccc24)S3)cc1]

12. The sirtuin modulating compound of claim 8, wherein the compound is a sirtuin activator.

13. The sirtuin modulating compound of claim 8, wherein the compound is a sirtuin inhibitor.

14. The sirtuin modulating compound of claim 8, wherein the compound modulates SIRT3 activity.

15. A sirtuin modulating compound of Formula XI and/or a salt thereof: 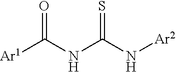
wherein Ar¹ and Ar² are independently selected from aryl and heteroaryl, wherein the aryl and heteroaryl are optionally substituted with one or more substituents selected from the group consisting of (C₁-C₁₀)-alkyl, (C₁-C₁₀)-alkenyl, alkoxy, halo, amine, sulfonyl-alkyl, acyl, amide, acetylamine, alkyl amine, alkynyl, and hydroxyl.

16. A sirtuin modulating compound of Formula V and/or a salt thereof: 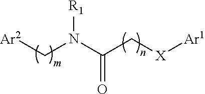
wherein R₁ is selected from the group consisting of hydrogen, alkyl, heteroalkyl, cycloalkyl, alkenyl, alkynyl, acyl, alkoxy, alkenyloxy, cycloalkyloxy, cycloalkenyl, and heterocycloalkyl; and wherein Ar¹ and Ar² are independently selected from aryl and heteroaryl, wherein the aryl and heteroaryl are optionally substituted with one or more substituents selected from the group consisting of (C₁-C₁₀)-alkyl, (C₁-C₁₀)-alkenyl, alkoxy, halo, amine, sulfonyl-alkyl, and hydroxyl; and wherein X is selected from the group consisting of O, S and N; and wherein m and n are integers each having a value independently selected from 0 to 10.

17. The sirtuin modulating compound of claim 16, wherein Formula V and/or salt thereof is:

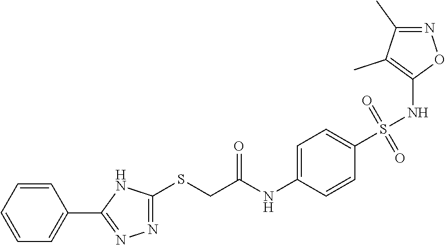 [SMILES: Cc1noc(NS(=O)(=O)c2ccc(NC(=O)CSc3nnc(-c4ccccc4)[nH]3)cc2)c1C]

18. The sirtuin modulating compound of claim 16, wherein Formula V and/or salt thereof is:

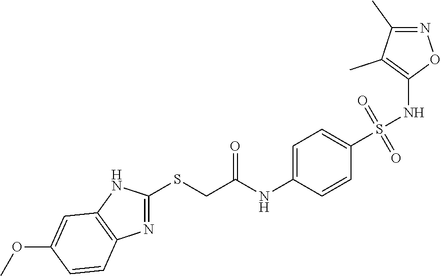 [SMILES: COc1ccc2nc(SCC(=O)Nc3ccc(S(=O)(=O)Nc4onc(C)c4C)cc3)[nH]c2c1]

19. The sirtuin modulating compound of claim 16, wherein Formula V and/or salt thereof is:

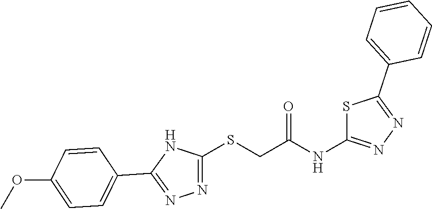 [SMILES: COc1ccc(-c2nnc(SCC(=O)Nc3nnc(-c4ccccc4)s3)[nH]2)cc1]

20. The sirtuin modulating compound of claim 16, wherein the compound is a sirtuin activator.

21. The sirtuin modulating compound of claim 16, wherein the compound is a sirtuin inhibitor.

22. The sirtuin modulating compound of claim 16, wherein the compound modulates SIRT3 activity.
